(19) United States
(12) Patent Application Publication
(10) Pub. No.: US 2022/0395472 A1
(43) Pub. Date: Dec. 15, 2022

(54) REMITTIVE EFFECTS OF TAPINAROF IN THE TREATMENT OF PLAQUE PSORIASIS, ATOPIC DERMATITIS, OR RADIATION DERMATITIS

(71) Applicant: Dermavant Sciences GmbH, Basel (CH)

(72) Inventors: David Scott RUBENSTEIN, Chapel Hill, NC (US); Kimberly Ann MCHALE, Durham, NC (US); Stephen C. PISCITELLI, Hillsborough, NC (US)

(21) Appl. No.: 17/820,692

(22) Filed: Aug. 18, 2022

Related U.S. Application Data
(63) Continuation-in-part of application No. 17/674,577, filed on Feb. 17, 2022.
(60) Provisional application No. 63/266,535, filed on Jan. 7, 2022.
(60) Provisional application No. 63/261,523, filed on Sep. 23, 2021.
(60) Provisional application No. 63/260,322, filed on Aug. 17, 2021.
(60) Provisional application No. 63/150,204, filed on Feb. 17, 2021.

(51) Int. Cl. A61K 31/05; A61K 9/00; A61K 9/06; A61P 17/06

(52) U.S. Cl. CPC A61K 31/05; A61K 9/0014; A61K 9/06; A61P 17/06

(57) ABSTRACT

Embodiments described herein are directed to methods for treating moderate to severe plaque psoriasis, atopic dermatitis, or radiation dermatitis in a subject comprising administering about 1.0% tapinarof in a topical composition to the affected areas of the subject once a day for an initial period of time until the plaque psoriasis, atopic dermatitis, or radiation dermatitis is clear at which time the administration is stopped, and reassessing the subject from about 3 months to about 12 months after administration is stopped to determine if further treatment is required. Also described is the remittive effect observed after once daily administration of 1% tapinarof cream.

### CROSS-REFERENCE TO RELATED APPLICATIONS

This application is a continuation-in-part of U.S. application Ser. No. 17/674,577 filed Feb. 17, 2022, which claims priority to and benefit of U.S. Provisional Application No. 63/150,204 filed Feb. 17, 2021, U.S. Provisional Application No. 63/260,322 filed Aug. 17, 2021, U.S. Provisional Application No. 63/261,523 filed Sep. 23, 2021, and U.S. Provisional Application No. 63/266,535 filed Jan. 7, 2022, the entire contents of each of which are hereby incorporated by reference.

### FIELD OF THE INVENTION

The present invention relates to methods of treating various chronic skin disorders and methods of achieving remission of the same.

### SUMMARY

Embodiments of the invention are directed to methods for treating plaque psoriasis (PsO) in a subject in need thereof, comprising:

a. applying a thin layer of about 1.0% tapinarof topical cream composition to the affected areas of the subject once a day for an initial period of time of at least 12 weeks, until the subject has a PGA score of 0, and a PASI score < the baseline PASI score and a DLQI score < the baseline DLQI score;

b. after the initial period of time, stop treating the subject with tapinarof for a remittive period of time of about 1 to about 7 months, wherein the remittive period of time is the time wherein the subject maintains a PGA score <2; and

c. if, after the remittive period of time, the subject has a PGA score of ≥2 and the PASI score and DLQI score is ≥ the PASI score and DLQI score in step a. further applying a thin layer of 1.0% tapinarof topical cream composition to the affected areas of the subject once a day for a further period of time, until the subject has a PGA score of 0;

wherein

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage my psoriasis with the study drug; 2) The time spent applying the study drug every day was acceptable and did not affect my everyday life; 3) I am satisfied with how well the study drug worked for my psoriasis; 4) I have confidence in the study drug; 5) The study drug cleared my skin and kept my psoriasis from coming back; 6) If the study drug was available by prescription, I would recommend it to other patients with psoriasis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my psoriasis; 14) The study drug is easier to use than other topical drugs I have used to treat my psoriasis; 15) I prefer the study drug to other topical drugs I have used to treat my psoriasis; 16) The study drug is more effective than systemic drugs I have used to treat my psoriasis; 17) The study drug is easier to use than systemic drugs I have used to treat my psoriasis; and 18) I prefer the study drug to systemic drugs I have used to treat my psoriasis.

In certain embodiments the plaque psoriasis is moderate or severe plaque psoriasis. In certain embodiments the initial period of time is about 12 weeks to about 52 weeks. In certain embodiments the remittive period is greater than 3 months and up to about 7 months. In certain embodiments the remittive period is about 4 months. In certain embodiments the further period of time is less than the initial period of time. In certain embodiments the subject selects a satisfaction rating of “agree” or “strongly agree” to three or more of the satisfaction questions. In certain embodiments the subject selects a satisfaction rating of “agree” or “strongly agree” to five or more of the satisfaction questions. In certain embodiments the subject selects a satisfaction rating of “agree” or “strongly agree” to ten or more of the satisfaction questions.

Embodiments of the invention are directed to a method for treating plaque psoriasis in a subject in need thereof, wherein the subject has achieved a PGA score of 0, a PASI score < their baseline PASI score, and a DLQI score < the baseline DLQI score, after applying a thin layer of about 1.0% tapinarof topical cream composition to the affected areas of the subject once a day for an initial period of time of about 12 weeks to 52 weeks, comprising:

a. stopping treatment of the subject with tapinarof for a remittive period of time of about 1 to about 7 months, wherein the remittive period of time is the time wherein the subject has a PGA score <2, and

b. if, after the remittive period of time, the subject has a PGA score of >2 and the PASI score and DLQI score is > the PASI score and DLQI score in step a.; further applying a thin layer of 1.0% tapinarof topical cream composition to the affected areas of the subject once a day for a further period of time until the subject has a PGA score of 0;

wherein

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of:

1) I can easily manage my psoriasis with the study drug; 2) The time spent applying the study drug every day was acceptable and did not affect my everyday life; 3) I am satisfied with how well the study drug worked for my psoriasis; 4) I have confidence in the study drug; 5) The study drug cleared my skin and kept my psoriasis from coming back; 6) If the study drug was available by prescription, I would recommend it to other patients with psoriasis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my psoriasis; 14) The study drug is easier to use than other topical drugs I have used to treat my psoriasis; 15) I prefer the study drug to other topical drugs I have used to treat my psoriasis; 16) The study drug is more effective than systemic drugs I have used to treat my psoriasis; 17) The study drug is easier to use than systemic drugs I have used to treat my psoriasis; and 18) I prefer the study drug to systemic drugs I have used to treat my psoriasis.

In certain embodiments the plaque psoriasis is moderate or severe plaque psoriasis. In certain embodiments the initial period of time is about 12 weeks to about 52 weeks. In certain embodiments the remittive period is greater than 3 months and up to about 7 months. In certain embodiments the remittive period is about 4 months. In certain embodiments the further period of time is less than the initial period of time. In certain embodiments the subject selects a satisfaction rating of “agree” or “strongly agree” to three or more of the satisfaction questions. In certain embodiments the subject selects a satisfaction rating of “agree” or “strongly agree” to five or more of the satisfaction questions.

Embodiments of the invention are directed to a method for treating plaque psoriasis in a subject in need thereof, wherein the subject has a PGA score of at least 1 point lower than baseline but has not achieved a PGA score of 0 after applying a thin layer of about 1.0% tapinarof topical cream composition to the affected areas of the subject once a day for a period of time of about 12 weeks to about 52 weeks comprising continuing to applying a thin layer of about 1.0% tapinarof topical cream composition to the affected areas of the subject once a day for an indefinite period of time, wherein the PGA score does not increase when applying a thin layer of about 1.0% tapinarof topical cream composition to the affected areas of the subject once a day;

wherein

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage my psoriasis with the study drug; 2) The time spent applying the study drug every day was acceptable and did not affect my everyday life; 3) I am satisfied with how well the study drug worked for my psoriasis; 4) I have confidence in the study drug; 5) The study drug cleared my skin and kept my psoriasis from coming back; 6) If the study drug was available by prescription, I would recommend it to other patients with psoriasis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my psoriasis; 14) The study drug is easier to use than other topical drugs I have used to treat my psoriasis; 15) I prefer the study drug to other topical drugs I have used to treat my psoriasis; 16) The study drug is more effective than systemic drugs I have used to treat my psoriasis; 17) The study drug is easier to use than systemic drugs I have used to treat my psoriasis; and 18) I prefer the study drug to systemic drugs I have used to treat my psoriasis.

In certain embodiments the plaque psoriasis is moderate or severe plaque psoriasis. In certain embodiments the subject selects a satisfaction rating of “agree” or “strongly agree” to three or more of the satisfaction questions. In certain embodiments the subject selects a satisfaction rating of “agree” or “strongly agree” to five or more of the satisfaction questions.

Embodiments of the invention are directed to a method for treating atopic dermatitis (AD) in a subject in need thereof, comprising:

a. applying a thin layer of about 1.0% tapinarof topical cream composition to the affected areas of the subject once a day for an initial period of time of at least 8 weeks, until the subject has an IGA score of 0;

b. after the initial period of time, stop treating the subject with tapinarof for a remittive period of time of about 1 month to about 7 months, wherein the remittive period of time is the time wherein the subject maintains an IGA score <2; and

c. if, after the remittive period of time, the subject has an IGA score of >2, further applying a thin layer of 1.0% tapinarof topical cream composition to the affected areas of the subject once a day for a further period of time, until the subject has an IGA score of 0;

wherein:

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

In certain embodiments, the atopic dermatitis is moderate or severe atopic dermatitis. In some embodiments, the initial period of time is about 12 weeks to about 52 weeks. In certain embodiments, the remittive period is greater than 3 months and up to about 7 months. In an embodiment, the remittive period is about 4 months. In some embodiments, wherein the further period of time is less than the initial period of time. In certain embodiments, the subject selects a satisfaction rating of “agree” or “strongly agree” to three or more of the satisfaction questions. In some embodiments, the subject selects a satisfaction rating of “agree” or “strongly agree” to five or more of the satisfaction questions. In certain embodiments, the subject selects a satisfaction rating of “agree” or “strongly agree” to ten or more of the satisfaction questions.

Embodiments of the invention are directed to a method for treating atopic dermatitis (AD) in a subject in need thereof, wherein the subject has achieved an IGA score of 0 after applying a thin layer of about 1.0% tapinarof topical cream composition to the affected areas of the subject once a day for an initial period of time of about 8 weeks to 52 weeks, comprising:

stopping treatment of the subject with tapinarof for a remittive period of time of about 1 month to about 7 months, wherein the remittive period of time is the time wherein the subject has an IGA score <2, and

if, after the remittive period of time, the subject has an IGA score of ≥2, further applying a thin layer of 1.0% tapinarof topical cream composition to the affected areas of the subject once a day for a further period of time until the subject has an IGA score of 0;

wherein:

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

In certain embodiments, the atopic dermatitis is moderate or severe atopic dermatitis. In some embodiments, the remittive period is greater than 3 months and up to about 7 months. In an embodiment, the remittive period is about 4 months. In certain embodiments, the further period of time is less than the initial period of time. In some embodiments, the subject selects a satisfaction rating of “agree” or “strongly agree” to three or more of the satisfaction questions. In certain embodiments, the subject selects a satisfaction rating of “agree” or “strongly agree” to five or more of the satisfaction questions.

Embodiments of the invention are directed to a method for treating atopic dermatitis (AD) in a subject in need thereof, wherein the subject has an IGA score of at least 1 point lower than baseline but has not achieved an IGA score of 0 after applying a thin layer of about 1.0% tapinarof topical cream composition to the affected areas of the subject once a day for a period of time of about 8 weeks to about 52 weeks, comprising continuing to apply a thin layer of about 1.0% tapinarof topical cream composition to the affected areas of the subject once a day for an indefinite period of time, wherein the IGA score does not increase when applying a thin layer of about 1.0% tapinarof topical cream composition to the affected areas of the subject once a day;

wherein:

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

In certain embodiments, the atopic dermatitis is moderate or severe atopic dermatitis. In some embodiments, the subject selects a satisfaction rating of “agree” or “strongly agree” to three or more of the satisfaction questions. In certain embodiments, the subject selects a satisfaction rating of “agree” or “strongly agree” to five or more of the satisfaction questions.

### DESCRIPTION OF DRAWINGS

FIG. 1 provides a graph of the dosing over time.

FIG. 2 provides a graph of tapinarof and tapinarof sulfate (a metabolite of tapinarof) concentrations over time.

FIG. 3 depicts the phase 3 study design.

FIG. 4 shows the long-term extension of tapinarof treatment.

FIG. 5 presents the time to first disease worsening among patients entering with complete disease clearance (PGA=0) i.e., the remittive effect.

FIG. 6 presents the durability of the response up to 52 weeks.

FIG. 7 presents a depiction of the potential mechanism of clinical remittive effect with tapinarof.

FIG. 8 is an example of 1 representative target lesion of 1 tapinarof-treated patient from PSOARING 1 clinical trial. PGA and PASI are global efficacy assessments. DLQI, Dermatology Life Quality Index; PASI, Psoriasis Area and Severity Index; PGA, Physician Global Assessment; PP-NRS, Peak Pruritus Numeric Rating Scale.

FIG. 9 displays improvements in a patient's clinical response for PGA, PASI, and DLQI during treatment with tapinarof cream in the pivotal (PSOARING 1) and LTE (PSOARING 3) trails.

FIG. 10 displays improvements in clinical response of a patient with plaque psoriasis treated with tapinarof cream and the remittive effect that persisted while off therapy for 24 weeks during the LTE trial.

FIG. 11 is a graph showing the mean change in % BSA during the LTE trial. Tapinarof-tapinarof subjects received tapinarof treatment during the pivotal trail and continued treatment during the LTE trail. Vehicle-tapinarof subjects received vehicle treatment during the pivotal trail and transitioned to tapinarof treatment at the start of the LTE trail.

FIG. 12 is a graph showing the change in PASI score by visit during the LTE trial. Tapinarof-tapinarof subjects received tapinarof treatment during the pivotal trail and continued treatment during the LTE trail. Vehicle-tapinarof subjects received vehicle treatment during the pivotal trail and transitioned to tapinarof treatment at the start of the LTE trail.

FIG. 13 is a graph showing the mean DLQI scores of the two groups during the LTE trial. Tapinarof-tapinarof subjects received tapinarof treatment during the pivotal trail and continued treatment during the LTE trail. Vehicle-tapinarof subjects received vehicle treatment during the pivotal trail and transitioned to tapinarof treatment at the start of the LTE trail

FIG. 14 is a graph showing the results of the Patient Satisfaction Questionnaire for Confidence and Satisfaction with the Efficacy of Tapinarof (n=599).

FIG. 15 is a graph showing the results of the Patient Satisfaction Questionnaire for Ease of Application and Cosmetic Elegance (n=599).

FIG. 16 is a graph showing the results of the Patient Satisfaction Questionnaire for patient preference for tapinarof versus previous topical therapies.

FIG. 17 is a graph showing the results of the Patient Satisfaction Questionnaire for patient preference for tapinarof versus previous systemic therapies.

FIG. 18 is a graph showing the patient reported tolerability scores for tapinarof cream during the course of the LTE trial.

FIGS. 19A-G are graphs showing the mean investigator-assessed irritation scores for sensitive skin areas treated with tapinarof cream during the course of the LTE trial, FIG. 19A anal crux, FIG. 19B axillae, FIG. 19C face, FIG. 19D neck, FIG. 19E genitalia, FIG. 19F skin folds, and FIG. 19G inframammary areas.

FIG. 20 is a graph showing the change in IGA score by dosing regimen over time in a Phase 2b study of subjects with atopic dermatitis, where treatment success was defined as an IGA score of clear or almost clear with a minimum 2-point improvement.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “subject” includes a single subject as well as two or more subjects; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a topical composition to a subject.

The term “applying a thin layer”, refers to rubbing enough of a composition (e.g., a 1% tapinarof cream composition) into the skin to cover the area requiring application until any residual cream is no longer visible.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The terms “controlled,” “control,” “clear,” or “clearance,” when referring to the treatment of the symptoms of plaque psoriasis shall mean the symptoms are negligible or non-existent, i.e. PGA is 0 or 1. The terms “controlled,” “control,” “clear,” or “clearance,” when referring to the treatment of the symptoms of atopic dermatitis (AD), shall mean the symptoms are negligible or non-existent, i.e. IGA is 0 or 1. The terms “controlled,” “control,” “clear,” or “clearance,” when referring to the treatment of the symptoms of radiation dermatitis shall mean the symptoms are negligible or non-existent, i.e. Grade 1 classification.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The term “patient” and “subject” are interchangeable and may be taken to mean any human which may be treated with compounds of the present invention. In some embodiments, the patient or subject is an adult, adolescent, child or infant. In some embodiments, the patient or subject is an adolescent (i.e. 12-17 years old). In some embodiments, the patient or subject is 18 years old or older. In some embodiments, the patient or subject is between the age of 18 and 75.

The term “remittive effect” when referring to the treatment of plaque psoriasis refers to the time during which treatment with tapinarof has stopped and plaque psoriasis remains controlled for a period of time after cessation of treatment. Plaque psoriasis is controlled when the PGA score is 0 or 1. The term “remission” means that plaque psoriasis in itself is not completely cured, but the symptoms thereof are temporarily or perpetually alleviated or have disappeared, the symptoms may be measured by PGA, PASI, BSA, DLQI, and Patient Satisfaction questionnaire.

The term “remittive effect” when referring to the treatment of atopic dermatitis refers to the time during which treatment with tapinarof has stopped and atopic dermatitis remains controlled for a period of time after cessation of treatment. Atopic dermatitis is controlled when the IGA score is 0 or 1. The term “remission” means that atopic dermatitis in itself is not completely cured, but the symptoms thereof are temporarily or perpetually alleviated or have disappeared, the symptoms may be measured by IGA, Itch/Pruritus, EASI, BSA, VAS for sleep or itch, and patient reported outcomes.

The term “remittive effect” when referring to the treatment of radiation dermatitis refers to the time during which treatment with tapinarof has stopped and radiation dermatitis remains controlled for a period of time after cessation of treatment. Radiation dermatitis is controlled when the classification is measured as Grade 1.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition. The term “treatment,” as used within the context of the present disclosure, is meant to include therapeutic treatment, as well as prophylactic or suppressive measures, for the treatment of psoriasis, atopic dermatitis, or radiation dermatitis. For example, the term treatment may include administration of tapinarof prior to or following the onset of psoriasis, atopic dermatitis, or radiation dermatitis thereby preventing or removing signs of the disease or disorder. As another example, administration of tapinarof after clinical manifestation of psoriasis, atopic dermatitis, or radiation dermatitis to combat the symptoms and/or complications of said disorders comprises “treatment” of the disease. In one embodiment, treatment of psoriasis in a subject comprises achieving remission of psoriasis in a subject. In one embodiment, treatment of psoriasis in a subject comprises inducing and maintaining remission of psoriasis in a subject. In another embodiment, treatment of psoriasis in a subject comprises maintaining remission of psoriasis in a subject. In one embodiment, treatment of atopic dermatitis in a subject comprises achieving remission of atopic dermatitis in a subject. In one embodiment, treatment of atopic dermatitis in a subject comprises inducing and maintaining remission of atopic dermatitis in a subject. In another embodiment, treatment of atopic dermatitis in a subject comprises maintaining remission of atopic dermatitis in a subject. In one embodiment, treatment of radiation dermatitis in a subject comprises achieving remission of radiation dermatitis in a subject. In one embodiment, treatment of radiation dermatitis in a subject comprises inducing and maintaining remission of radiation dermatitis in a subject. In another embodiment, treatment of radiation dermatitis in a subject comprises maintaining remission of radiation dermatitis in a subject.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Psoriasis is a common, chronic relapsing inflammatory skin disease with recurrent episodes of prominently erythematous and scaly patches (plaques). Approximately 2 to 3% of the global population is affected by psoriasis; those affected are predominantly adults, who are most often diagnosed between the ages of 18 to 35 years. Psoriasis disrupts daily activities such as work and/or school attendance, interpersonal relationships, recreational activities, and intimacy, thereby significantly impacting sufferers' quality of life. Furthermore, psoriasis sufferers can also have co-morbidities such as arthritis, depression, inflammatory bowel disease, and cardiovascular (CV) diseases.

Up to 80% of patients have mild to moderate plaque-type psoriasis, which is generally managed with topical treatments. Topically-applied corticosteroids and Vitamin D analogs, alone or in combination, are the most commonly used products in the treatment of psoriasis. Vitamin D analogs are moderately efficacious as monotherapy, while application of topical corticosteroids—particularly the very potent ones—is restricted in terms of body areas that can be treated and the duration of use due to the well-known application site and systemic adverse drug reactions.

Although numerous topical treatment options are available, there still remains a need for a topical treatment that combines a high level of efficacy with an acceptable safety profile that permits application to a large body surface area (BSA) without restrictions on duration of treatment.

Tapinarof, also known as DMVT-505 and formerly known as GSK2894512, WBI-1001, or benvitimod, is a fully synthetic hydroxylated stilbene, new molecular entity and is a novel non-steroidal anti-inflammatory agent for the topical treatment of atopic dermatitis (AD), radiation dermatitis, and plaque psoriasis.

Psoriatic skin lesions and atopic dermatitis skin lesions contain elevated numbers of activated T-cells, which have a key role in the pathogenesis of inflammatory diseases through the proliferation and secretion of pro-inflammatory cytokines. Radiation dermatitis skin lesions contain an inflammatory infiltrate which consists of increased numbers and/or activation of inflammatory cells including, but not limited to T-cells and/or B-cells and/or neutrophils and/or antigen presenting cells and/or other cell lineages generally described as granulocytes and/or lymphocytes and/or macrophages. The drug likely mediates its effects via the aryl hydrocarbon receptor (AhR) agonist and nuclear factor erythroid 2-related factor 2 (Nrf2) because the pattern of pro-inflammatory mediators inhibited by tapinarof is different from that of corticosteroids, calcineurin inhibitors, vitamin D analogs, and other immunosuppressive agents commonly used to treat AD and psoriasis. Rather, the profile of biological responses elicited by tapinarof most closely matches that of the dual activation properties of coal tar, a common nonprescription treatment for psoriasis. Together, existing data identify tapinarof as a non-steroid, therapeutic AhR-modulating agent (TAMA), which is a unique mechanism of action compared with existing therapies. The efficacy of tapinarof in these inflammatory skin diseases is attributed to tapinarof binding to and activating the ligand-dependent transcription factor aryl hydrocarbon receptor (AhR), resulting in the downregulation of proinflammatory cytokines, restoration of the skin barrier through modulation of skin barrier protein expression, and regulation of gene expression in immune cells.

Clinical trials, described herein, have demonstrated a remittive effect with topical tapinarof therapy, defined as maintenance of disease control off therapy. In the Phase 2b clinical trial in patients with psoriasis, significant improvements were maintained for four weeks following the discontinuation of tapinarof treatment; this remittive effect was confirmed in the Phase 3 psoriasis program in which patients entering the long-term extension study with complete disease clearance (Physician Global Assessment [PGA] score=0) maintained disease control off therapy for approximately 4 months before requiring retreatment with tapinarof (PGA score ≥2).

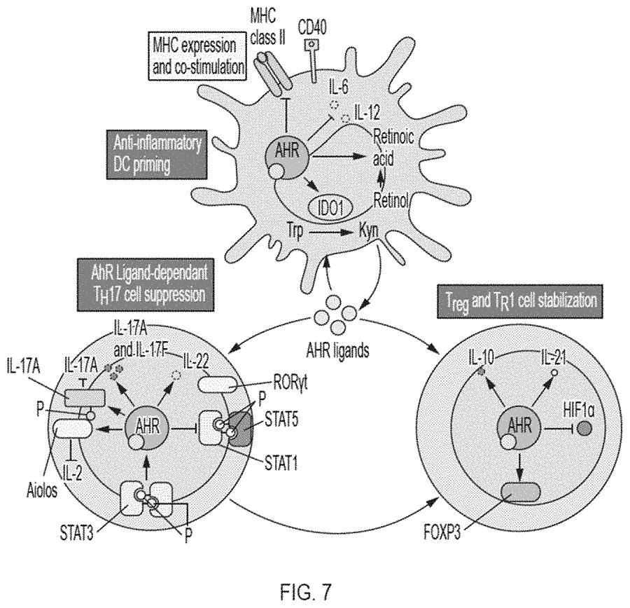

The observed remittive effect of tapinarof cream in patients with inflammatory skin diseases, including psoriasis and atopic dermatitis, results from tapinarof's binding to the ligand-dependent transcription factor aryl hydrocarbon receptor (AhR). Specifically, binding of tapinarof to AhR modulates T cell responses both through intrinsic control of T cell subset differentiation and via controlling the function of antigen presenting cells. Tapinarof modulated AhR signaling (i) promotes the conversion of Th17 cells to type 1 regulatory T cells, (ii) more generally regulates T cell polarization, T cell tolerance, T cell anergy, and/or T cell exhaustion, (iii) regulates T regulatory cell differentiation, polarization of T cells, and/or the generation of resident memory T cells, and (iv) regulates the balance of TH17 and type 1 regulatory T cells, (iii) regulates antigen presenting cell (APC) development, maturation, polarization, activation, and/or blockade of APC:T-cell interactions. More specifically, the efficacy of tapinarof is attributed to its specific binding and activation of AhR. This leads to the downregulation of pro-inflammatory Th17 cytokines (including interleukin IL-17A and IL-17F), increase in expression of skin barrier proteins related to keratinocyte differentiation, including filaggrin and loricrin, increase in antioxidant activity, and regulation of gene expression in immune cells. AhR is widely expressed in immune cells, including antigen-presenting cells (APCs), T cells, macrophages, mast cells, and other skin immune cells. In APCs, including Langerhans and dendritic cells (DCs), AhR expression is necessary for function and cytokine expression. Tapinarof-AhR has been shown to directly downregulate IL-17A and IL-17F, which likely explains its immediate therapeutic benefit in PsO. In addition, the remittive effect observed off therapy in the PSOARING trial program may be explained by the additional roles of AhR including modulation of T-cell responses through both intrinsic control of T-cell subset differentiation and via control of APCs' function that may contribute to the clinical remittive effect observed. AhR signaling via canonical and non-canonical pathways drives multiple tolerogenic mechanisms in DCs (FIG. 7): AhR dampens expression of major histocompatibility complex class II expression in APCs, It alters production of cytokines involved in differentiation of effector and regulatory T cells (Tregs), AhR signaling in DCs controls expression of metabolites with immunoregulatory function, in particular, AhR promotes the expression of indoleamine 2,3-dioxygenase, an enzyme that catalyzes the production of kynurenine, itself an AhR agonist, which promotes FoxP3+ T-cell differentiation and suppresses inflammation in many contexts. Additionally, the specific binding and activation of AhR by tapinarof has been shown to inhibit T-cell expansion and Helper T cell (Th) 17-cell differentiation and reduce IL-17 production in T-cell assays. Ligand-dependent AhR activation has also been demonstrated to result in epigenetic modification of both the FoxP3 and IL-17 gene promoters leading to preferential differentiation of Tregs and inhibition of Th17 cells.

As these pathways are probed experimentally, further refinement/specific descriptions of cell (T-cell, APC) subset/differentiation are likely to be elucidated/described providing a clearer mechanism of action, i.e., subsets of tissue resident memory cells may be described by a combination of antigen biomarkers including CD45ROhi, CD45RAlow, CD69hi, CD103hi and CD103low.

It was surprisingly and unexpectedly found that after treatment with 1% tapinarof cream formulation topically administered once daily for about 3 months the symptoms of plaque psoriasis remained improved in subjects not being treated for greater than 4 months. Though a similar trial (Cai et al., Chinese Medical Journal, 2020; 133(24)) observed improvement in symptoms after treatment had stopped, that trial dosed the subjects with a 1% tapinarof formulation twice a day. The 1% tapinarof cream formulation described herein was only applied once a day, therefore it was completely unexpected that application of half of the amount of tapinarof would lead to a remittive effect for a period of time greater than 3 months.

The tapinarof topical composition described herein does not show a tachyphylaxis effect as seen with other active ingredients. Tachyphylaxis is the decrease in response to successive doses of a drug, rendering it less effective. Such a surprising and unexpected characteristic of the tapinarof topical composition described herein allows for the continuous daily administration of the composition to the skin as well as the use in large body surface areas without concern for decreased efficacy over time and the potential increase of side effects as seen with other treatments. Without wishing to be bound by theory, the beneficial lack of tachyphlaxis is not due to an active metabolite, is not a characteristic of vehicle cream alone, nor is it an artifact of patient non-compliance. Accordingly, the tapinarof topical composition described herein can continue to be administered to a patient in need thereof at the same dose and dose frequency to continue to achieve at least the same efficacy, if not improved efficacy, over time without increasing the risk of any potential side effects over time. COMPOSITIONS

Embodiments of the invention are directed to topical compositions comprising 3,5-Dihydroxy-4-isopropyl-trans-stilbene or a pharmaceutically acceptable salt thereof. Throughout this disclosure 3,5-Dihydroxy-4-isopropyl-trans-stilbene is referred to as tapinarof, and is also known as (E)-2-isopropyl-5-styrylbenzene-1,3 diol, with the empirical formula C17H1802, a molecular weight of 254.32, and the following structure:

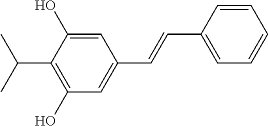 [SMILES: CC(C)c1c(O)cc(/C=C/c2ccccc2)cc1O]

In embodiments, the topical composition is an emulsion. In embodiments, the topical composition is an oil-in-water emulsion. In embodiments, the topical composition is an oil-in-water emulsion cream having a viscosity of about 20,000 to 100,000 centipoise (cps), preferably about 20,000 to 60,000 cps and more preferably about 30,000 to 40,000 cps; a D50 of less than or equal to about 1 μm, preferably about 0.1 μm to about 0.6 μm, and more preferably about 0.2 μm to about 0.3 μm; and a D90 of about 0.2 μm to about 1.5 μm, preferably about 0.4 μm to about 1 μm, and more preferably about 0.5 μm to about 0.6 μm of the oil droplets. In an embodiment, the topical composition of tapinarof or a pharmaceutically acceptable salt thereof, comprises an oil phase, and a water phase, creating an emulsion, and wherein the emulsion composition is homogenous. In an embodiment, tapinarof, or pharmaceutically acceptable salt thereof is solubilized in the oil phase of the emulsion composition. In embodiments, the oil phase is comprised of medium chain triglycerides, propylene glycol, non-ionic emulsifying wax, diethylene glycol monoethyl ether, polyoxyl stearyl ether-2, polysorbate 80, polyoxyl stearyl ether-20, benzoic acid, and butylated hydroxytoluene. In embodiments, the water phase is comprised of sodium citrate, edetate disodium, citric acid monohydrate, and water.

In certain embodiments described herein, the topical composition comprises about 1.0% tapinarof, or a pharmaceutically acceptable salt thereof. The tapinarof topical pharmaceutical oil-in-water emulsion compositions described in U.S. Pat. No. 10,195,160, U.S. Publication No. 2018/0064656, and PCT Application No. PCT/US2021/038794 are each incorporated herein in its entirety.

In certain embodiments described herein, the topical composition comprises about 0.05% to about 2% 3,5-dihydroxy-4-isopropyl-trans-stilbene or a pharmaceutically acceptable salt thereof, about 2% to about 30% of an oil phase, about 55% to about 75% of an aqueous phase, about 1% to about 20% of a surfactant, and a dermatologically acceptable excipient selected from the group consisting of an antioxidant, a pH adjusting agent, a chelating agent, a preservative, a co-solvent and combinations thereof. In certain embodiments, the oil phase comprises medium chain triglycerides of a carbon length from six to twelve. In certain embodiments, the aqueous phase comprises water. In certain embodiments, the surfactant is selected from the group consisting of a non-ionic emulsifying wax NF, steareth-2, steareth-20, polysorbate 80, and combinations thereof. In certain embodiments described herein, the topical composition comprises about 50.00% to about 75.00% water, about 0.05% to about 0.50% sodium citrate, about 0.01% to about 2.00% citric acid, about 0.01% to about 1.00% disodium EDTA, about 5.00% to about 25.00% propylene glycol, about 0.10% to about 5.00% diethylene glycol monoethyl ether, about 0.01% to about 1.00% butylated hydroxytoluene, about 0.01% to about 1.00% benzoic acid, about 5.00% to about 10.00% emulsifying wax, about 5.00% to about 25.00% medium chain triglycerides (MCT), about 0.50% to about 5.00% polysorbate 80, about 0.50% to about 5.00% steareth 2, and about 0.50% to about 5.00% steareth 20. In preferred embodiments, the topical composition comprises 1.00% tapinarof, or a pharmaceutically acceptable salt thereof, 64.68% water, 0.19% sodium citrate, 0.08% citric acid, 0.10% disodium EDTA, 10.00% propylene glycol, 2.00% diethylene glycol monoethyl ether, 0.10% butylated hydroxytoluene, 0.25% benzoic acid, 7.20% emulsifying wax, 10.00% medium chain triglycerides (MCT), 1.50% polysorbate 80, 1.80% steareth 2, and 1.10% steareth 20. In another embodiment, the emulsifying wax is a proprietary blend known as “Polawax NF” (Registered Trademark) (Croda Inc, Edison, N.J., USA).

### Methods of Using Topical Compositions to Treat Psoriasis

Embodiments of the invention are directed to methods for treating plaque psoriasis in a subject in need thereof, comprising:

a. administering about 1.0% tapinarof topical cream composition to the affected areas of the subject once a day for an initial period of time of at least 12 weeks, until the subject has a PGA score of 0, and a PASI score < the baseline PASI score and a DLQI score < the baseline DLQI score;

b. after the initial period of time, stop treating the subject with tapinarof for a remittive period of time of about 1 to about 7 months, wherein the remittive period of time is the time wherein the subject maintains a PGA score <2; and

c. if, after the remittive period of time, the subject has a PGA score of ≥2 and the PASI score and DLQI score is ≥ the PASI score and DLQI score in step a. further administering 1.0% tapinarof topical cream composition to the affected areas of the subject once a day for a further period of time, until the subject has a PGA score of 0;

wherein

the 1.0% tapinarof topical cream composition does not prolong the QTc interval in the subject while applying the 1.0% tapinarof topical cream; and

the plasma concentration of tapinarof in the subject is below 50 pg/mL while applying the 1.0% tapinarof topical cream.

Embodiments of the invention are directed to methods for treating plaque psoriasis in a subject in need thereof, comprising:

a. administering about 1.0% tapinarof topical cream composition to the affected areas of the subject once a day for an initial period of time of at least 12 weeks, until the subject has a PGA score of 0, and a PASI score < the baseline PASI score and a DLQI score < the baseline DLQI score;

b. after the initial period of time, stop treating the subject with tapinarof for a remittive period of time of about 1 to about 7 months, wherein the remittive period of time is the time wherein the subject maintains a PGA score <2; and

c. if, after the remittive period of time, the subject has a PGA score of ≥2 and the PASI score and DLQI score is ≥ the PASI score and DLQI score in step a. further administering 1.0% tapinarof topical cream composition to the affected areas of the subject once a day for a further period of time, until the subject has a PGA score of 0;

wherein

the 1.0% tapinarof topical cream composition does not prolong the QTc interval in the subject while applying the 1.0% tapinarof topical cream; and

the plasma concentration of tapinarof in the subject is below 50 pg/mL while applying the 1.0% tapinarof topical cream.

Embodiments of the invention are directed to methods for treating plaque psoriasis in a subject in need thereof, comprising:

a. applying a thin layer of about 1.0% tapinarof topical cream composition to the affected areas of the subject once a day for an initial period of time of at least 12 weeks, until the subject has a PGA score of 0, and a PASI score < the baseline PASI score and a DLQI score < the baseline DLQI score;

b. after the initial period of time, stop treating the subject with tapinarof for a remittive period of time of about 1 to about 7 months, wherein the remittive period of time is the time wherein the subject maintains a PGA score <2; and

c. if, after the remittive period of time, the subject has a PGA score of ≥2 and the PASI score and DLQI score is ≥ the PASI score and DLQI score in step a. further applying a thin layer of 1.0% tapinarof topical cream composition to the affected areas of the subject once a day for a further period of time, until the subject has a PGA score of 0;

wherein

the 1.0% tapinarof topical cream composition does not prolong the QTc interval in the subject while applying the 1.0% tapinarof topical cream; and

the plasma concentration of tapinarof in the subject is below 50 pg/mL while applying the 1.0% tapinarof topical cream.

Embodiments of the invention are directed to methods for treating plaque psoriasis in a subject in need thereof, comprising:

a. applying a thin layer of about 1.0% tapinarof topical cream composition to the affected areas of the subject once a day for an initial period of time of at least 12 weeks, until the subject has a PGA score of 0, and a PASI score < the baseline PASI score and a DLQI score < the baseline DLQI score;

b. after the initial period of time, stop treating the subject with tapinarof for a remittive period of time of about 1 to about 7 months, wherein the remittive period of time is the time wherein the subject maintains a PGA score <2; and

c. if, after the remittive period of time, the subject has a PGA score of ≥2 and the PASI score and DLQI score is ≥ the PASI score and DLQI score in step a. further applying a thin layer of 1.0% tapinarof topical cream composition to the affected areas of the subject once a day for a further period of time, until the subject has a PGA score of 0;

wherein

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage my psoriasis with the study drug; 2) The time spent applying the study drug every day was acceptable and did not affect my everyday life; 3) I am satisfied with how well the study drug worked for my psoriasis; 4) I have confidence in the study drug; 5) The study drug cleared my skin and kept my psoriasis from coming back; 6) If the study drug was available by prescription, I would recommend it to other patients with psoriasis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my psoriasis; 14) The study drug is easier to use than other topical drugs I have used to treat my psoriasis; 15) I prefer the study drug to other topical drugs I have used to treat my psoriasis; 16) The study drug is more effective than systemic drugs I have used to treat my psoriasis; 17) The study drug is easier to use than systemic drugs I have used to treat my psoriasis; and 18) I prefer the study drug to systemic drugs I have used to treat my psoriasis.

In certain embodiments the plaque psoriasis is moderate or severe plaque psoriasis.

In certain embodiments the initial period of time is about 12 weeks to about 52 weeks. In certain embodiments the initial period of time is about 12 weeks. In certain embodiments the initial period of time is about 16, about 24, about 28, about 32, about 36, about 40, about 44, about 48 or about 52 weeks. In certain embodiments the initial period of time is 16-40 weeks. In certain embodiments the initial period of time is 40 weeks. In certain embodiments the initial period is 52 weeks.

In certain embodiments the remittive period is greater than 3 months and up to about 7 months. In certain embodiments the remittive period of time is about 4 months, about 5 months, about 6 months or about 7 months. In certain embodiments the remittive period is about 4 months.

In certain embodiments the further period of time is less than the initial period of time. In certain embodiments the further period of time is about 8 weeks to 16 weeks. In certain embodiments the further period of time is about 8 weeks, about 9 weeks, about 10 weeks, about 11 weeks, about 12 weeks, about 13 weeks, about 14 weeks, about 15 weeks, or about 16 weeks.

In certain embodiments the subject selects a satisfaction rating of “agree” or “strongly agree” to two or more of the satisfaction questions. In certain embodiments the subject selects a satisfaction rating of “agree” or “strongly agree” to three or more of the satisfaction questions. In certain embodiments the subject selects a satisfaction rating of “agree” or “strongly agree” to four or more of the satisfaction questions. In certain embodiments the subject selects a satisfaction rating of “agree” or “strongly agree” to five or more of the satisfaction questions. In certain embodiments the subject selects a satisfaction rating of “agree” or “strongly agree” to six or more of the satisfaction questions. In certain embodiments the subject selects a satisfaction rating of “agree” or “strongly agree” to seven or more of the satisfaction questions. In certain embodiments the subject selects a satisfaction rating of “agree” or “strongly agree” to eight or more of the satisfaction questions. In certain embodiments the subject selects a satisfaction rating of “agree” or “strongly agree” to nine or more of the satisfaction questions. In certain embodiments the subject selects a satisfaction rating of “agree” or “strongly agree” to ten or more of the satisfaction questions. In certain embodiments the subject selects a satisfaction rating of “agree” or “strongly agree” to eleven or more of the satisfaction questions. In certain embodiments the subject selects a satisfaction rating of “agree” or “strongly agree” to twelve or more of the satisfaction questions. In certain embodiments the subject selects a satisfaction rating of “agree” or “strongly agree” to thirteen or more of the satisfaction questions. In certain embodiments the subject selects a satisfaction rating of “agree” or “strongly agree” to fourteen or more of the satisfaction questions. In certain embodiments the subject selects a satisfaction rating of “agree” or “strongly agree” to fifteen or more of the satisfaction questions. In certain embodiments the subject selects a satisfaction rating of “agree” or “strongly agree” to sixteen or more of the satisfaction questions. In certain embodiments the subject selects a satisfaction rating of “agree” or “strongly agree” to seventeen or more of the satisfaction questions. In certain embodiments the subject selects a satisfaction rating of “agree” or “strongly agree” to eighteen or more of the satisfaction questions.

Embodiments of the invention are directed to a method for treating plaque psoriasis in a subject in need thereof, wherein the subject has achieved a PGA score of 0, a PASI score < their baseline PASI score, and a DLQI score < the baseline DLQI score, after applying a thin layer of about 1.0% tapinarof topical cream composition to the affected areas of the subject once a day for an initial period of time of about 12 weeks to 52 weeks, comprising:

a. stopping treatment of the subject with tapinarof for a remittive period of time of about 1 to about 7 months, wherein the remittive period of time is the time wherein the subject has a PGA score <2, and

b. if, after the remittive period of time, the subject has a PGA score of ≥2 and the PASI score and DLQI score is ≥ the PASI score and DLQI score in step a.; further applying a thin layer of 1.0% tapinarof topical cream composition to the affected areas of the subject once a day for a further period of time until the subject has a PGA score of 0;

wherein

the 1.0% tapinarof topical cream composition does not prolong the QTc interval in the subject while applying the 1.0% tapinarof topical cream; and

the plasma concentration of tapinarof in the subject is below 50 pg/mL while applying the 1.0% tapinarof topical cream.

Embodiments of the invention are directed to a method for treating plaque psoriasis in a subject in need thereof, wherein the subject has achieved a PGA score of 0, a PASI score < their baseline PASI score, and a DLQI score < the baseline DLQI score, after administering about 1.0% tapinarof topical cream composition to the affected areas of the subject once a day for an initial period of time of about 12 weeks to 52 weeks, comprising:

a. stopping treatment of the subject with tapinarof for a remittive period of time of about 1 to about 7 months, wherein the remittive period of time is the time wherein the subject has a PGA score <2, and

b. if, after the remittive period of time, the subject has a PGA score of >2 and the PASI score and DLQI score is > the PASI score and DLQI score in step a.; further administering 1.0% tapinarof topical cream composition to the affected areas of the subject once a day for a further period of time until the subject has a PGA score of 0;

wherein

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of:

1) I can easily manage my psoriasis with the study drug; 2) The time spent applying the study drug every day was acceptable and did not affect my everyday life; 3) I am satisfied with how well the study drug worked for my psoriasis; 4) I have confidence in the study drug; 5) The study drug cleared my skin and kept my psoriasis from coming back; 6) If the study drug was available by prescription, I would recommend it to other patients with psoriasis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my psoriasis; 14) The study drug is easier to use than other topical drugs I have used to treat my psoriasis; 15) I prefer the study drug to other topical drugs I have used to treat my psoriasis; 16) The study drug is more effective than systemic drugs I have used to treat my psoriasis; 17) The study drug is easier to use than systemic drugs I have used to treat my psoriasis; and 18) I prefer the study drug to systemic drugs I have used to treat my psoriasis.

Embodiments of the invention are directed to a method for treating plaque psoriasis in a subject in need thereof, wherein the subject has achieved a PGA score of 0, a PASI score < their baseline PASI score, and a DLQI score < the baseline DLQI score, after applying a thin layer of about 1.0% tapinarof topical cream composition to the affected areas of the subject once a day for an initial period of time of about 12 weeks to 52 weeks, comprising:

a. stopping treatment of the subject with tapinarof for a remittive period of time of about 1 to about 7 months, wherein the remittive period of time is the time wherein the subject has a PGA score <2, and

b. if, after the remittive period of time, the subject has a PGA score of >2 and the PASI score and DLQI score is > the PASI score and DLQI score in step a.; further applying a thin layer of 1.0% tapinarof topical cream composition to the affected areas of the subject once a day for a further period of time until the subject has a PGA score of 0;

wherein

the 1.0% tapinarof topical cream composition does not prolong the QTc interval in the subject while applying the 1.0% tapinarof topical cream; and

the plasma concentration of tapinarof in the subject is below 50 pg/mL while applying the 1.0% tapinarof topical cream.

Embodiments of the invention are directed to a method for treating plaque psoriasis in a subject in need thereof, wherein the subject has achieved a PGA score of 0, a PASI score < their baseline PASI score, and a DLQI score < the baseline DLQI score, after applying a thin layer of about 1.0% tapinarof topical cream composition to the affected areas of the subject once a day for an initial period of time of about 12 weeks to 52 weeks, comprising:

a. stopping treatment of the subject with tapinarof for a remittive period of time of about 1 to about 7 months, wherein the remittive period of time is the time wherein the subject has a PGA score <2, and

b. if, after the remittive period of time, the subject has a PGA score of >2 and the PASI score and DLQI score is > the PASI score and DLQI score in step a.; further applying a thin layer of 1.0% tapinarof topical cream composition to the affected areas of the subject once a day for a further period of time until the subject has a PGA score of 0;

wherein

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of:

1) I can easily manage my psoriasis with the study drug; 2) The time spent applying the study drug every day was acceptable and did not affect my everyday life; 3) I am satisfied with how well the study drug worked for my psoriasis; 4) I have confidence in the study drug; 5) The study drug cleared my skin and kept my psoriasis from coming back; 6) If the study drug was available by prescription, I would recommend it to other patients with psoriasis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my psoriasis; 14) The study drug is easier to use than other topical drugs I have used to treat my psoriasis; 15) I prefer the study drug to other topical drugs I have used to treat my psoriasis; 16) The study drug is more effective than systemic drugs I have used to treat my psoriasis; 17) The study drug is easier to use than systemic drugs I have used to treat my psoriasis; and 18) I prefer the study drug to systemic drugs I have used to treat my psoriasis.

In certain embodiments the plaque psoriasis is moderate or severe plaque psoriasis.

In certain embodiments the initial period of time is about 12 weeks. In certain embodiments the initial period of time is 16-40 weeks. In certain embodiments the initial period of time is about 16, about 24, about 28, about 32, about 36, about 40, about 44, about 48 or about 52 weeks. In certain embodiments the initial period of time is 16-40 weeks. In certain embodiments the initial period of time is 40 weeks. In certain embodiments the initial period is 52 weeks.

In certain embodiments the remittive period is greater than 3 months and up to about 7 months. In certain embodiments the remittive period of time is about 4 months, about 5 months, about 6 months or about 7 months. In certain embodiments the remittive period is about 4 months.

In certain embodiments the further period of time is less than the initial period of time. In certain embodiments the further period of time is 8 to 16 weeks. In certain embodiments the further period of time is about 8 weeks, about 9 weeks, about 10 weeks, about 11 weeks, about 12 weeks, about 13 weeks, about 14 weeks, about 15 weeks, or about 16 weeks.

In certain embodiments the subject selects a satisfaction rating of “agree” or “strongly agree” to two or more of the satisfaction questions. In certain embodiments the subject selects a satisfaction rating of “agree” or “strongly agree” to three or more of the satisfaction questions. In certain embodiments the subject selects a satisfaction rating of “agree” or “strongly agree” to four or more of the satisfaction questions. In certain embodiments the subject selects a satisfaction rating of “agree” or “strongly agree” to five or more of the satisfaction questions. In certain embodiments the subject selects a satisfaction rating of “agree” or “strongly agree” to six or more of the satisfaction questions. In certain embodiments the subject selects a satisfaction rating of “agree” or “strongly agree” to seven or more of the satisfaction questions. In certain embodiments the subject selects a satisfaction rating of “agree” or “strongly agree” to eight or more of the satisfaction questions. In certain embodiments the subject selects a satisfaction rating of “agree” or “strongly agree” to nine or more of the satisfaction questions. In certain embodiments the subject selects a satisfaction rating of “agree” or “strongly agree” to ten or more of the satisfaction questions. In certain embodiments the subject selects a satisfaction rating of “agree” or “strongly agree” to eleven or more of the satisfaction questions. In certain embodiments the subject selects a satisfaction rating of “agree” or “strongly agree” to twelve or more of the satisfaction questions. In certain embodiments the subject selects a satisfaction rating of “agree” or “strongly agree” to thirteen or more of the satisfaction questions. In certain embodiments the subject selects a satisfaction rating of “agree” or “strongly agree” to fourteen or more of the satisfaction questions. In certain embodiments the subject selects a satisfaction rating of “agree” or “strongly agree” to fifteen or more of the satisfaction questions. In certain embodiments the subject selects a satisfaction rating of “agree” or “strongly agree” to sixteen or more of the satisfaction questions. In certain embodiments the subject selects a satisfaction rating of “agree” or “strongly agree” to seventeen or more of the satisfaction questions. In certain embodiments the subject selects a satisfaction rating of “agree” or “strongly agree” to eighteen or more of the satisfaction questions.

Embodiments of the invention are directed to a method for treating plaque psoriasis in a subject in need thereof, wherein the subject has a PGA score of at least 1 point lower than baseline but has not achieved a PGA score of 0 after administering 1.0% tapinarof topical cream composition to the affected areas of the subject once a day for a period of time of about 12 weeks to about 52 weeks comprising continuing to applying a thin layer of about 1.0% tapinarof topical cream composition to the affected areas of the subject once a day for an indefinite period of time, wherein the PGA score does not increase when administering about 1.0% tapinarof topical cream composition to the affected areas of the subject once a day; wherein

the 1.0% tapinarof topical cream composition does not prolong the QTc interval in the subject while applying the 1.0% tapinarof topical cream and

the plasma concentration of tapinarof in the subject is below 50 pg/mL while applying the 1.0% tapinarof topical cream.

Embodiments of the invention are directed to a method for treating plaque psoriasis in a subject in need thereof, wherein the subject has a PGA score of at least 1 point lower than baseline but has not achieved a PGA score of 0 after administering about 1.0% tapinarof topical cream composition to the affected areas of the subject once a day for a period of time of about 12 weeks to about 52 weeks comprising continuing to administering about 1.0% tapinarof topical cream composition to the affected areas of the subject once a day for an indefinite period of time, wherein the PGA score does not increase when applying a thin layer of about 1.0% tapinarof topical cream composition to the affected areas of the subject once a day;

wherein

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage my psoriasis with the study drug; 2) The time spent applying the study drug every day was acceptable and did not affect my everyday life; 3) I am satisfied with how well the study drug worked for my psoriasis; 4) I have confidence in the study drug; 5) The study drug cleared my skin and kept my psoriasis from coming back; 6) If the study drug was available by prescription, I would recommend it to other patients with psoriasis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my psoriasis; 14) The study drug is easier to use than other topical drugs I have used to treat my psoriasis; 15) I prefer the study drug to other topical drugs I have used to treat my psoriasis; 16) The study drug is more effective than systemic drugs I have used to treat my psoriasis; 17) The study drug is easier to use than systemic drugs I have used to treat my psoriasis; and 18) I prefer the study drug to systemic drugs I have used to treat my psoriasis.

Embodiments of the invention are directed to a method for treating plaque psoriasis in a subject in need thereof, wherein the subject has a PGA score of at least 1 point lower than baseline but has not achieved a PGA score of 0 after applying a thin layer of about 1.0% tapinarof topical cream composition to the affected areas of the subject once a day for a period of time of about 12 weeks to about 52 weeks comprising continuing to applying a thin layer of about 1.0% tapinarof topical cream composition to the affected areas of the subject once a day for an indefinite period of time, wherein the PGA score does not increase when applying a thin layer of about 1.0% tapinarof topical cream composition to the affected areas of the subject once a day;

wherein

the 1.0% tapinarof topical cream composition does not prolong the QTc interval in the subject while applying the 1.0% tapinarof topical cream; and

the plasma concentration of tapinarof in the subject is below 50 pg/mL while applying the 1.0% tapinarof topical cream.

Embodiments of the invention are directed to a method for treating plaque psoriasis in a subject in need thereof, wherein the subject has a PGA score of at least 1 point lower than baseline but has not achieved a PGA score of 0 after applying a thin layer of about 1.0% tapinarof topical cream composition to the affected areas of the subject once a day for a period of time of about 12 weeks to about 52 weeks comprising continuing to applying a thin layer of about 1.0% tapinarof topical cream composition to the affected areas of the subject once a day for an indefinite period of time, wherein the PGA score does not increase when applying a thin layer of about 1.0% tapinarof topical cream composition to the affected areas of the subject once a day;

wherein

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage my psoriasis with the study drug; 2) The time spent applying the study drug every day was acceptable and did not affect my everyday life; 3) I am satisfied with how well the study drug worked for my psoriasis; 4) I have confidence in the study drug; 5) The study drug cleared my skin and kept my psoriasis from coming back; 6) If the study drug was available by prescription, I would recommend it to other patients with psoriasis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my psoriasis; 14) The study drug is easier to use than other topical drugs I have used to treat my psoriasis; 15) I prefer the study drug to other topical drugs I have used to treat my psoriasis; 16) The study drug is more effective than systemic drugs I have used to treat my psoriasis; 17) The study drug is easier to use than systemic drugs I have used to treat my psoriasis; and 18) I prefer the study drug to systemic drugs I have used to treat my psoriasis.

In certain embodiments the plaque psoriasis is moderate or severe plaque psoriasis.

In certain embodiments the subject selects a satisfaction rating of “agree” or “strongly agree” to two or more of the satisfaction questions. In certain embodiments the subject selects a satisfaction rating of “agree” or “strongly agree” to three or more of the satisfaction questions. In certain embodiments the subject selects a satisfaction rating of “agree” or “strongly agree” to four or more of the satisfaction questions. In certain embodiments the subject selects a satisfaction rating of “agree” or “strongly agree” to five or more of the satisfaction questions. In certain embodiments the subject selects a satisfaction rating of “agree” or “strongly agree” to six or more of the satisfaction questions. In certain embodiments the subject selects a satisfaction rating of “agree” or “strongly agree” to seven or more of the satisfaction questions. In certain embodiments the subject selects a satisfaction rating of “agree” or “strongly agree” to eight or more of the satisfaction questions. In certain embodiments the subject selects a satisfaction rating of “agree” or “strongly agree” to nine or more of the satisfaction questions. In certain embodiments the subject selects a satisfaction rating of “agree” or “strongly agree” to ten or more of the satisfaction questions. In certain embodiments the subject selects a satisfaction rating of “agree” or “strongly agree” to eleven or more of the satisfaction questions. In certain embodiments the subject selects a satisfaction rating of “agree” or “strongly agree” to twelve or more of the satisfaction questions. In certain embodiments the subject selects a satisfaction rating of “agree” or “strongly agree” to thirteen or more of the satisfaction questions. In certain embodiments the subject selects a satisfaction rating of “agree” or “strongly agree” to fourteen or more of the satisfaction questions. In certain embodiments the subject selects a satisfaction rating of “agree” or “strongly agree” to fifteen or more of the satisfaction questions. In certain embodiments the subject selects a satisfaction rating of “agree” or “strongly agree” to sixteen or more of the satisfaction questions. In certain embodiments the subject selects a satisfaction rating of “agree” or “strongly agree” to seventeen or more of the satisfaction questions. In certain embodiments the subject selects a satisfaction rating of “agree” or “strongly agree” to eighteen or more of the satisfaction questions.

Embodiments described herein are directed to methods for developing a remittive effect in a subject with moderate to severe plaque psoriasis comprising administering about 1.0% tapinarof in a topical composition to the affected areas of the subject once a day for an initial period of time until the plaque psoriasis is clear at which time the administration is stopped, and reassessing the subject from about 3 months to about 12 months after administration is stopped to determine if further treatment is required.

Embodiments described herein are directed to methods for treating moderate to severe plaque psoriasis in a subject comprising administering about 1.0% tapinarof in a topical composition to the affected areas of the subject once a day for an initial period of time until the plaque psoriasis is clear at which time the administration is stopped, and reassessing the subject from about 3 months to about 12 months after administration is stopped to determine if further treatment is required. In embodiments described herein, the topical composition is administered in an initial period of time. In embodiments described herein, the initial period of time is about 16 weeks to about 40 weeks in duration. In certain embodiments, the initial period of time is about 40 weeks.

In embodiments described herein, reassessment of the subject occurs at about 4 months after administration. In certain embodiments, further treatment is initiated after reassessment of the subject. In certain embodiments, the further treatment comprises administering about 1.0% tapinarof in a topical composition to the affected areas of the subject once a day for a subsequent period of time until the plaque psoriasis is clear.

Embodiments described herein are directed to methods for inducing a remittive effect in a subject with moderate to severe plaque psoriasis, wherein the remittive effect can be temporary or permanent.

Embodiments described herein are directed to methods for achieving remission in a subject with moderate to severe plaque psoriasis comprising administering about 1.0% tapinarof in a topical composition to the affected areas of the subject once a day until the subject reaches a PGA score of 0, wherein administration of the about 1.0% tapinarof in a topical composition is stopped. In certain embodiments, the subject reaches a PGA score of 0 after about 4 weeks, 8 weeks or 12 weeks of treatment with the 1% Tapinarof topical composition once a day. In certain embodiments, once the subject reaches a PGA score of 0, the administration is stopped for about 4 months, 5 months, 6 months, 7 months, 8 months, 9 months, 10 months, 11 months, or indefinitely. In certain embodiments, treatment is reinitiated when the subject reaches a PGA score of ≥2, wherein treatment is continued until the subject achieves a PGA score of 0.

Embodiments described herein are directed to methods for treating a subject with moderate to severe plaque psoriasis who has achieved a PGA score of 0 after about 4 weeks, 8 weeks or 12 weeks of administration of about 1.0% tapinarof in a topical composition comprising stopping administration of the about 1.0% tapinarof in a topical composition for about 4 months, 5 months, 6 months, 7 months, 8 months, 9 months, 10 months, 11 months, or indefinitely.

Embodiments described herein are directed to methods for achieving remission in a subject with moderate to severe plaque psoriasis comprising administering about 1.0% tapinarof in a topical composition to the affected areas of the subject having a PGA score of ≥1 once a day, wherein administration of the about 1.0% tapinarof in a topical composition is stopped when the subject reaches a PGA score of 0. In certain embodiments, the subject reaches a PGA score of 0 after about 4 weeks, 8 weeks or 12 weeks of treatment. In certain embodiments, once the subject reaches a PGA score of 0 the administration is stopped for about 4 months, 5 months, 6 months, 7 months, 8 months, 9 months, 10 months, 11 months, or indefinitely. In certain embodiments, treatment is reinitiated when the subject reaches a PGA score of ≥2, wherein treatment is continued until the subject achieves a PGA score of 0.

Embodiments described herein are directed to methods for maintaining remission of moderate to severe plaque psoriasis in a subject comprising administering about 1.0% tapinarof in a topical composition to the affected areas of the subject once a day for an initial period of time until the plaque psoriasis is clear at which time the administration is stopped and remission is achieved, wherein the plaque psoriasis is clear is defined as one or more symptom is alleviated.

Embodiments described herein are directed to methods for inducing remission of moderate to severe plaque psoriasis in a subject comprising administering about 1.0% tapinarof in a topical composition to the affected areas of the subject once a day for an initial period of time until the plaque psoriasis is clear at which time the administration is stopped and remission has been induced, wherein the plaque psoriasis is clear is defined as one or more symptom is alleviated.

In embodiments described herein, the treating moderate to severe plaque psoriasis in a subject is wherein one or more symptom of psoriasis is improved. In embodiments described herein, the one or more symptom of moderate to severe psoriasis is measured according to an assessment selected from Physician Global Assessment (PGA) score, Psoriasis Area and Severity Index (PASO, target lesion grading, percent body surface area (BSA) affected, Dermatology Quality of Life Index (DLQI), or Patient Satisfaction questionnaire. In certain embodiments, plaque psoriasis is treated when the Physician Global Assessment (PGA) score is 0 or 1. In certain embodiments, plaque psoriasis is treated when the Physician Global Assessment (PGA) score has improved from baseline by at least 2-grades and as described below. In certain embodiments, plaque psoriasis is treated when the Psoriasis Area and Severity Index (PASO has improved from baseline by a decrease in total PASI score and as described below. In certain embodiments, plaque psoriasis is treated when the target lesion grading has improved from baseline and as described below. In certain embodiments, plaque psoriasis is treated when the percent body surface area (BSA) affected has improved from baseline and as described below. In certain embodiments, plaque psoriasis is treated when the Dermatology Quality of Life Index (DLQI) has improved from baseline and as described below. In certain embodiments, plaque psoriasis is treated when the Patient Satisfaction questionnaire has improved from baseline and as described below.

In embodiments described herein, reassessment of the subject results in further treatment when a new nidus (previous application site or other site) or aggravating psoriasis (such as erythema, scale, immersion) occurs. In embodiments described herein, reassessment of the subject results in further treatment when compared with baseline and the symptoms are no longer improved or clear.

In embodiments described herein, further treatment is required if the PGA score is greater than or equal to 2. In embodiments described herein, further treatment is required if the PGA score is greater than or equal to 3. In embodiments described herein, further treatment is required if the PGA score is greater than or equal to 4. In embodiments described herein, further treatment is required if the Psoriasis Area and Severity Index (PASO returns to baseline. In embodiments described herein, further treatment is required if the target lesion grading returns to baseline. In embodiments described herein, further treatment is required if the percent body surface area (BSA) affected returns to baseline. In embodiments described herein, further treatment is required if the Dermatology Quality of Life Index (DLQI) returns to baseline. In embodiments described herein, further treatment is required if the Patient Satisfaction questionnaire returns to baseline.

In embodiments described herein, further treatment comprises administering about 1.0% tapinarof in a topical composition to the affected areas of the subject once a day for a subsequent period of time until the plaque psoriasis is treated. In certain embodiments, the subsequent period of time is about 8 weeks to about 16 weeks. In certain embodiments, the subsequent period of time can be up to 52 weeks.

Embodiments of the invention are directed to methods of treating moderate to severe plaque psoriasis in a subject comprising topically administering to the subject in need thereof a topical composition containing tapinarof as described herein, wherein skin type does not affect the efficacy of the treatment. In some embodiments, the skin type is measured using the Fitzpatrick scale, wherein the subject's skin type is selected from the group consisting of Fitzpatrick skin type I, Fitzpatrick skin type II, Fitzpatrick skin type III, Fitzpatrick skin type IV, Fitzpatrick skin type V, and Fitzpatrick skin type VI. Fitzpatrick scale is a numerical classification schema for human skin color. The following list shows the six categories of the Fitzpatrick scale: Type I—always burns, never tans (palest, freckles); Type II—usually burns, tans minimally; Type III—sometimes mild burn, tans uniformly; Type IV—burns minimally, always tans well (moderate brown); Type V—very rarely burns, tans very easily (dark brown); or Type VI—never burns (deeply pigmented dark brown to darkest brown).

In embodiments described herein, the topically administering of the topical composition includes application to the skin of the body, arms, legs, back, chest, buttocks, neck, scalp, fingernails, or toenails where the moderate to severe plaque psoriasis lesions are present (or “affected area”). The topically administering of the topical composition includes applying enough of the topical composition to completely cover each lesion with a thin layer. In embodiments described herein, administration of the topical composition requires that the subject lightly rub the cream into the skin until it is no longer visible.

In embodiments described herein, the subject has been diagnosed with moderate to severe plaque psoriasis and has had stable disease for at least 6 months, at least 7 months, at least 8 months, at least 9 months, at least 10 months, at least 11 months, or at least 12 months.

In embodiments described herein, the subject is younger than 18 years of age. In embodiments described herein, the subject is 18 years of age or older. In embodiments described herein, the subject is between the ages of 18 to 65 years old. In embodiments described herein, the subject is between the ages of 18 to 75 years old.

In embodiments described herein, the topical composition is administered once daily for up to 52 weeks. In embodiments described herein, the topical composition is administered once daily for about 52 weeks. In embodiments described herein, the topical composition is administered once daily for up to 48 weeks. In embodiments described herein, the topical composition is administered once daily for about 48 weeks. In embodiments described herein, the topical composition is administered once daily for up to 44 weeks. In embodiments described herein, the topical composition is administered once daily for about 44 weeks. In embodiments described herein, the topical composition is administered once daily for up to 40 weeks. In embodiments described herein, the topical composition is administered once daily for about 40 weeks. In embodiments described herein, the topical composition is administered once daily for up to 36 weeks. In embodiments described herein, the topical composition is administered once daily for about 36 weeks. In embodiments described herein, the topical composition is administered once daily for up to 32 weeks. In embodiments described herein, the topical composition is administered once daily for about 32 weeks. In embodiments described herein, the topical composition is administered once daily for up to 28 weeks. In embodiments described herein, the topical composition is administered once daily for about 28 weeks. In embodiments described herein, the topical composition is administered once daily for up to 24 weeks. In embodiments described herein, the topical composition is administered once daily for about 24 weeks. In embodiments described herein, the topical composition is administered once daily for up to 12 weeks. In embodiments described herein, the topical composition is administered once daily for about 12 weeks. In embodiments described herein, the topical composition is administered once daily for up to 8 weeks. In embodiments described herein, the topical composition is administered once daily for about 8 weeks. In embodiments described herein, the topical composition is administered once daily for up to 6 weeks. In embodiments described herein, the topical composition is administered once daily for about 6 weeks. In embodiments described herein, the topical composition is administered once daily for up to 4 weeks. In embodiments described herein, the topical composition is administered once daily for about 4 weeks. In embodiments described herein, the topical composition is administered once daily until the moderate to severe plaque psoriasis is resolved.

In certain embodiments, the duration of treatment success (time off treatment) will correlate with the severity of baseline disease. For example, a subject with a baseline PGA score of 3 may have treatment success for about 4 weeks to about 12 weeks, a subject with a baseline PGA score of 2 may have treatment success for about 8 weeks to about 20 weeks, a subject with a baseline PGA score of 1 may have treatment success for about 12 weeks to about 24 weeks, and a subject with a baseline PGA score of 0 may have treatment success for about 20 weeks to about 52 weeks.

In embodiments described herein, the subject has been diagnosed with moderate to severe plaque psoriasis having a Physician Global Assessment (PGA) score of about 2, about 3, or about 4. In embodiments described herein, the subject has been diagnosed with moderate to severe mild to moderate plaque psoriasis having a Physician Global Assessment (PGA) score of greater than or equal to 2. A PGA score of 2 is a diagnosis of mild plaque psoriasis. A PGA score of 3 is a diagnosis of moderate plaque psoriasis. A PGA score of 4 is a diagnosis of severe plaque psoriasis. In embodiments described herein, the Physician Global Assessment (PGA) is used to assess the current state/severity of a subject's psoriasis. It is a static 5-point morphological assessment of overall disease severity, as determined by the investigator, using the clinical characteristics of erythema, plaque thickness, and scaling as guidelines. In certain embodiments, the PGA is assessed daily, weekly, or monthly and without reference to previous scores. The scoring system includes: Score of 0 represents clear skin with no signs of psoriasis, post-inflammatory hyperpigmentation may be present; Score of 1 represents almost clear skin with no thickening, normal to pink coloration, no to minimal focal scaling; Score of 2 represents mild psoriasis with just detectable to mild thickening, pink to light red coloration, predominantly fine scaling; Score of 3 represents moderate psoriasis with clearly distinguishable to moderate thickening, dull to bright red, clearly distinguishable erythema, moderate scaling; and Score of 4 represents severe psoriasis with severe thickening with hard edges, bright to deep dark red coloration, severe/coarse scaling covering almost all or all lesions. In embodiments described herein, the subject's Physician Global Assessment (PGA) score improved by about 1 grade, about 2 grades, about 3 grades, about 4 grades, or about 5 grades. In embodiments described herein, the subject's Physician Global Assessment (PGA) score improved by about 2 grades. In embodiments described herein, the subject's Physician Global Assessment (PGA) score improved to a score of about 0 or clear. In embodiments described herein, the subject's Physician Global Assessment (PGA) score improved to a score of about 1 or almost clear. In certain embodiments, the subject achieved a score of about 0 or about 1 by week 1, week 2, week 3, week 4, week 5, week 6, week 7, week 8, week 9, week 10, week 11, or week 12. In certain embodiments, the subject achieved a 2-grade improvement by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of PGA score after treatment has ended. In embodiments described herein, the subject has sustained improvement of PGA score about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of PGA score about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In certain embodiments, the subject achieved a PGA score of 0 or 1 at least once over a 52 week period of time. In certain embodiments, the subject achieved a PGA score of 0 at least once over a 52 week period of time. In certain embodiments, the subject achieved a PGA score of 0 or 1 at least once over a 44 week period of time. In certain embodiments, the subject achieved a PGA score of 0 at least once over a 44 week period of time. In certain embodiments, the subject did not experience worsening during a 52 week period of time.

In embodiments described herein, administration of the topical composition to a subject having severe plaque psoriasis is effectively treated wherein the subject achieved a “Clear” or “Almost Clear” rating according to the PGA with at least a 2 point improvement. In embodiments described herein, administration of the topical composition to a subject having moderate plaque psoriasis is effectively treated wherein the subject achieved a “Clear” or “Almost Clear” rating according to the PGA with at least a 2 point improvement. In embodiments described herein, administration of the topical composition to a subject having mild plaque psoriasis is effectively treated wherein the subject achieved a “Clear” rating according to the PGA.

In embodiments described herein, the subject has been diagnosed with moderate to severe plaque psoriasis having a percent body surface area (BSA) affected of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%. In preferred embodiments, the subject has been diagnosed with moderate to severe plaque psoriasis having a percent body surface area (BSA) affected of about 3% to about 20%. In embodiments described herein, body surface area (BSA) excludes the scalp, palms of the hands and soles of the feet. The assessment of the % BSA affected is an estimate of the percentage of total involved skin with psoriasis. The extent of BSA affected by psoriasis is a general indicator of disease severity. In embodiments described herein, one percent body surface area (1% BSA) is the equivalent of the total palmar surface of the palm plus 5 digits. The % BSA affected is calculated using the following regional body areas: Head and neck; Trunk, includes internal axillae and groin; Upper extremities, includes arms, external axillae, and hands; and Lower extremities, includes legs, buttocks, and feet. In embodiments described herein, body surface area (BSA) excludes the scalp, palms of the hands and soles of the feet. The % BSA assessment is utilized in the PASI. The % BSA affected by psoriasis is evaluated from 0 to 100%. In embodiments described herein, the subject's percent body surface area (BSA) affected is decreased. In certain embodiments, the subject achieved a decrease in the % BSA affected by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of % BSA affected after treatment has ended. In embodiments described herein, the subject has sustained improvement of percent body surface area (BSA) affected about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of % BSA affected about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the Psoriasis Area and Severity Index (PASI) is used to assess the severity of psoriasis that takes into account the overall severity of erythema (redness), thickness (induration), and scale (desquamation), as well as the extent of BSA affected with psoriasis. The 3 clinical signs are each graded on a 5 point scale (0 to 4) and the % BSA affected is scored on a 7-point scale (0 to 6) for each of the 4 specified body regions (head, upper extremities, trunk, and lower extremities). The individual scores are multiplied by a weighted factor for each body region; the sum of these scores gives the overall PASI score. Higher scores indicate more severe disease. PASI is a static assessment made without reference to previous scores. In embodiments described herein, the subject's Psoriasis Area and Severity Index (PASI) is improved by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In embodiments described herein, the subject's Psoriasis Area and Severity Index (PASI) is improved by greater than or equal to 25%, greater than or equal to 50%, greater than or equal to 75%, or greater than or equal to 90%. In embodiments described herein, the subject's Psoriasis Area and Severity Index (PASI) is improved by greater than or equal to 50%. In embodiments described herein, the subject's Psoriasis Area and Severity Index (PASI) is improved by greater than or equal to 75%. In embodiments described herein, the subject's Psoriasis Area and Severity Index (PASI) is improved by greater than or equal to 90%. In certain embodiments, the subject achieved a greater than 50% improvement in PASI by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In certain embodiments, the subject achieved a greater than 75% improvement in PASI by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In certain embodiments, the subject achieved a greater than 90% improvement in PASI by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of PASI after treatment has ended. In embodiments described herein, the subject has sustained improvement of PASI about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of PASI about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the target lesion grading is determined by measuring a single target lesion at baseline to assess efficacy in treating a discrete area rather than an overall average of all areas. For that single target lesion, the severity of erythema, scaling, and plaque thickness is assessed on a 5-point scale ranging from 0 (=none) to 4 (=severe). The maximum score was 15, with higher scores indicating more severe disease. In embodiments described herein, the subject's target lesion grading improved by about 1 point, about 2 points, 3 points, about 4 points, about 5 points, about 6 points, about 7 points, about 8 points, about 9 points, about 10 points, about 11 points, about 12 points, about 13 points, about 14 points, or about 15 points. In certain embodiments, the subject achieved an improvement in target lesion grading by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of target lesion grading after treatment has ended. In embodiments described herein, the subject has sustained improvement of target lesion grading about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of target lesion grading about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, subjects record changes using the Dermatology Quality of Life Index (DLQI) questionnaire. The DLQI is a simple dermatology-specific 10 question validated questionnaire to assess the impact of the disease on a subject's quality of life. The DLQI has become an important outcome measure in dermatology clinical trials and is the most frequently used instrument in studies of randomized controlled trials in dermatology. The DLQI can be analyzed as a total score (where a higher score indicates greater impairment in quality of life) and can also be scored for the following dimensions: Symptoms and Feelings (items 1 and 2), Daily Activities (items 3 and 4), Leisure (items 5 and 6), Work and School (item 7), Personal Relationships (items 8 and 9), and Treatment (item 10). In certain embodiments, the subject reported an improvement on the impact of one or more daily activities assessed by the DLQI by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement on the impact of one or more daily activities assessed by the DLQI after treatment has ended. In embodiments described herein, the subject has sustained improvement of one or more daily activities assessed by the DLQI about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. IV. In embodiments described herein, the subject does not experience worsening of one or more daily activities assessed by the DLQI about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the Patient Satisfaction questionnaire includes the questions provided below, wherein the subject selects one of the following: Strongly Agree, Agree, Neutral, Disagree, or Strongly Disagree.
1. I can easily manage my psoriasis with the study drug
2. The time spent applying the study drug every day was acceptable and did not affect my everyday life
3. I am satisfied with how well the study drug worked for my psoriasis
4. I have confidence in the study drug
5. The study drug cleared my skin and kept my psoriasis from coming back
6. If the study drug was available by prescription, I would recommend it to other patients with psoriasis
7. If the study drug was available by a prescription, I would use it again or continue on it
8. The study drug is easy to apply
9. The study drug is not greasy
10. The study drug quickly absorbs into my skin
11. The study drug feels good on my skin
12. I am satisfied with the look and feel of the study drug
The below questions #13-18 are comparing the study drug to other drugs used to treat psoriasis in the past. A topical drug is a drug that is applied to the skin like creams, ointments, lotions, gels, foams, sprays. A systemic drug is a drug that is taken by mouth like a capsule or tablet or injected through the skin with a needle like a shot or through a vein by a healthcare practitioner.
Subject is asked if they have used other topical drugs to treat psoriasis in the past? If yes, they answer questions 13-15. If no, they skip to question 16.
13. The study drug is more effective than other topical drugs I have used to treat my psoriasis
14. The study drug is easier to use than other topical drugs I have used to treat my psoriasis
15. I prefer the study drug to other topical drugs I have used to treat my psoriasis Subject is asked if they have used systemic drugs to treat psoriasis in the past?
16. The study drug is more effective than systemic drugs I have used to treat my psoriasis
17. The study drug is easier to use than systemic drugs I have used to treat my psoriasis
18. I prefer the study drug to systemic drugs I have used to treat my psoriasis

In embodiments described herein, the one or more symptoms improved by about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after administering the topical composition. In embodiments described herein, the one or more symptoms improved by about 2 weeks of administering the topical composition. In embodiments described herein, the one or more symptoms improved by about 4 weeks of administering the topical composition. In embodiments described herein, the one or more symptoms improved by about 8 weeks of administering the topical composition. In embodiments described herein, the one or more symptoms improved by about 12 weeks of administering the topical composition.

In some embodiments, it was surprisingly found that the topical composition may produce long lasting effects on the skin and may modify the long-term course of moderate to severe plaque psoriasis. Specifically, in certain embodiments, the improvement of the symptoms seen during administration of the topical composition may be maintained long after the final administration of the topical composition. In embodiments described herein, the one or more symptoms remain improved and the remittive effect lasted about 1 week, about 2 weeks, about 3 weeks, about 4 weeks, about 5 weeks, about 6 weeks, about 12 weeks, about 13 weeks, about 14 weeks, about 15 weeks, about 16 weeks, about 17 weeks, about 18 weeks, about 19 weeks, about 20 weeks, or up to 52 weeks after administration of the topical composition has ceased. In embodiments described herein, the one or more symptoms remain improved and the remittive effect lasted about 2 weeks after administration of the topical composition has ceased. In embodiments described herein, the one or more symptoms remain improved and the remittive effect lasted about 3 weeks after administration of the topical composition has ceased. In embodiments described herein, the one or more symptoms remain improved and the remittive effect lasted about 4 weeks after administration of the topical composition has ceased. In embodiments described herein, the one or more symptoms remain improved and the remittive effect lasted about 8 weeks after administration of the topical composition has ceased. In embodiments described herein, the one or more symptoms remain improved and the remittive effect lasted about 12 weeks after administration of the topical composition has ceased. In embodiments described herein, the one or more symptoms remain improved and the remittive effect lasted about 16 weeks after administration of the topical composition has ceased. In embodiments described herein, the one or more symptoms remain improved and the remittive effect lasted about 20 weeks after administration of the topical composition has ceased. In embodiments described herein, the one or more symptoms remain improved and the remittive effect lasted about 24 weeks after administration of the topical composition has ceased. In embodiments described herein, the one or more symptoms remain improved and the remittive effect lasted about 28 weeks after administration of the topical composition has ceased. In embodiments described herein, the one or more symptoms remain improved and the remittive effect lasted about 32 weeks after administration of the topical composition has ceased. In embodiments described herein, the one or more symptoms remain improved and the remittive effect lasted about 36 weeks after administration of the topical composition has ceased. In embodiments described herein, the one or more symptoms remain improved and the remittive effect lasted about 42 weeks after administration of the topical composition has ceased. In embodiments described herein, the one or more symptoms remain improved and the remittive effect lasted about 48 weeks after administration of the topical composition has ceased. In embodiments described herein, the one or more symptoms remain improved and the remittive effect lasted about 52 weeks after administration of the topical composition has ceased.

It was surprisingly found that the topical composition may produce long lasting effects on the skin and may modify the long-term course of moderate to severe plaque psoriasis. Specifically, in certain embodiments, the symptoms do not worsen after the final administration of the topical composition. In embodiments described herein, the one or more symptoms do not worsen about 1 week, about 2 weeks, about 3 weeks, about 4 weeks, about 5 weeks, about 6 weeks, about 12 weeks, about 13 weeks, about 14 weeks, about 15 weeks, about 16 weeks, about 17 weeks, about 18 weeks, about 19 weeks, about 20 weeks, or up to 52 weeks after administration of the topical composition has ceased. In embodiments described herein, the one or more symptoms do not worsen about 2 weeks after administration of the topical composition has ceased. In embodiments described herein, the one or more symptoms do not worsen about 3 weeks after administration of the topical composition has ceased. In embodiments described herein, the one or more symptoms do not worsen about 4 weeks after administration of the topical composition has ceased. In embodiments described herein, the one or more symptoms do not worsen about 8 weeks after administration of the topical composition has ceased. In embodiments described herein, the one or more symptoms do not worsen about 12 weeks after administration of the topical composition has ceased. In embodiments described herein, the one or more symptoms do not worsen about 16 weeks after administration of the topical composition has ceased. In embodiments described herein, the one or more symptoms do not worsen about 20 weeks after administration of the topical composition has ceased. In embodiments described herein, the one or more symptoms do not worsen about 24 weeks after administration of the topical composition has ceased. In embodiments described herein, the one or more symptoms do not worsen about 28 weeks after administration of the topical composition has ceased. In embodiments described herein, the one or more symptoms do not worsen about 32 weeks after administration of the topical composition has ceased. In embodiments described herein, the one or more symptoms do not worsen about 36 weeks after administration of the topical composition has ceased. In embodiments described herein, the one or more symptoms do not worsen about 42 weeks after administration of the topical composition has ceased. In embodiments described herein, the one or more symptoms do not worsen about 48 weeks after administration of the topical composition has ceased. In embodiments described herein, the one or more symptoms do not worsen about 52 weeks after administration of the topical composition has ceased.

In embodiments described herein, the topical composition can be used in a long-term treatment regimen, compared with topical corticosteroids which can only be used for 2-4 weeks. In embodiments described herein, the topical composition can be used for greater than 12 weeks.

Plaque psoriasis is a chronic disease which typically requires daily treatment for the duration of the subject's life. Available treatments, such are corticosteroids, have high side effects and cannot be administered for longer than 2 weeks at a time, after which time a rest phase from treatment must be taken before treatment can resume. Surprisingly, the tapinarof topical composition described herein is safe to use daily for an extended period of time which is greater than 2 weeks. In embodiments described herein, the tapinarof topical composition described herein can be continuously administered to the subject for greater than 2 weeks, greater than 1 month, greater than 2 months, greater than 3 months, greater than 4 months, or longer and during such period of administration, efficacy of the treatment is maintained (e.g., no loss of efficacy over time). Additionally, current treatments are only applied to the areas of the skin where there is an active lesion. Surprisingly, the tapinarof topical composition described herein can be safely applied to all areas of the skin without any limit as to total amount of body surface area receives treatment, for example, the subject can apply to active lesions as well as clear regions. In embodiments described herein, the tapinarof topical composition described herein can be administered to any or all of the following regions: back, elbows, knees, legs, soles of the feet, scalp, face, palms, genitals, or chest.

Embodiments described herein are directed to methods for treating a subject with moderate to severe plaque psoriasis comprising administering about 1.0% tapinarof in a topical composition to the affected areas of the subject once a day for an indefinite amount of time, wherein the subject reaches a PGA score of 1 or improves by at least 1 grade. In embodiments described herein, the subject maintains an improved PGA score but does not reach 0. In embodiments described herein, the subject achieves an improved PGA score of 0 after 4 months of administration of the tapinarof topical composition.

### Methods of Using Topical Compositions to Treat Atopic Dermatitis

Embodiments of the invention are directed to a method for treating atopic dermatitis (AD) in a subject in need thereof, comprising:

a. applying a thin layer of about 1.0% tapinarof topical cream composition to the affected areas of the subject once a day for an initial period of time of at least 8 weeks, until the subject has an IGA score of 0;

b. after the initial period of time, stop treating the subject with tapinarof for a remittive period of time of about 1 month to about 7 months, wherein the remittive period of time is the time wherein the subject maintains an IGA score <2; and

c. if, after the remittive period of time, the subject has an IGA score of >2, further applying a thin layer of 1.0% tapinarof topical cream composition to the affected areas of the subject once a day for a further period of time, until the subject has an IGA score of 0;

wherein:

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

Embodiments of the invention are directed to a method for treating atopic dermatitis (AD) in a subject in need thereof, wherein the subject has achieved an IGA score of 0 after applying a thin layer of about 1.0% tapinarof topical cream composition to the affected areas of the subject once a day for an initial period of time of about 8 weeks to 52 weeks, comprising:

stopping treatment of the subject with tapinarof for a remittive period of time of about 1 month to about 7 months, wherein the remittive period of time is the time wherein the subject has an IGA score <2, and

if, after the remittive period of time, the subject has an IGA score of ≥2, further applying a thin layer of 1.0% tapinarof topical cream composition to the affected areas of the subject once a day for a further period of time until the subject has an IGA score of 0;

wherein:

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

Embodiments of the invention are directed to a method for treating atopic dermatitis (AD) in a subject in need thereof, wherein the subject has an IGA score of at least 1 point lower than baseline but has not achieved an IGA score of 0 after applying a thin layer of about 1.0% tapinarof topical cream composition to the affected areas of the subject once a day for a period of time of about 8 weeks to about 52 weeks, comprising continuing to apply a thin layer of about 1.0% tapinarof topical cream composition to the affected areas of the subject once a day for an indefinite period of time, wherein the IGA score does not increase when applying a thin layer of about 1.0% tapinarof topical cream composition to the affected areas of the subject once a day;

wherein:

the 1.0% tapinarof topical cream composition does not prolong the QTc interval in the subject while applying the 1.0% tapinarof topical cream,

the plasma concentration of tapinarof in the subject is below 50 pg/mL while applying the 1.0% tapinarof topical cream, and

the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

In certain embodiments, the atopic dermatitis is moderate or severe atopic dermatitis.

In some embodiments, the initial period of time is about 8 weeks to about 52 weeks. In certain embodiments the initial period of time is about 8 weeks. In certain embodiments the initial period of time is about 12, about 16, about 24, about 28, about 32, about 36, about 40, about 44, about 48 or about 52 weeks. In certain embodiments the initial period of time is 16-40 weeks. In certain embodiments the initial period of time is 40 weeks. In certain embodiments the initial period is 52 weeks.

In certain embodiments, the remittive period is greater than 3 months and up to about 7 months. In certain embodiments the remittive period of time is about 4 months, about 5 months, about 6 months or about 7 months. In certain embodiments the remittive period is about 4 months.

In some embodiments, wherein the further period of time is less than the initial period of time. In certain embodiments the further period of time is about 8 weeks to 16 weeks. In certain embodiments the further period of time is about 8 weeks, about 9 weeks, about 10 weeks, about 11 weeks, about 12 weeks, about 13 weeks, about 14 weeks, about 15 weeks, or about 16 weeks.

In certain embodiments the subject selects a satisfaction rating of “agree” or “strongly agree” to two or more of the satisfaction questions. In certain embodiments the subject selects a satisfaction rating of “agree” or “strongly agree” to three or more of the satisfaction questions. In certain embodiments the subject selects a satisfaction rating of “agree” or “strongly agree” to four or more of the satisfaction questions. In certain embodiments the subject selects a satisfaction rating of “agree” or “strongly agree” to five or more of the satisfaction questions. In certain embodiments the subject selects a satisfaction rating of “agree” or “strongly agree” to six or more of the satisfaction questions. In certain embodiments the subject selects a satisfaction rating of “agree” or “strongly agree” to seven or more of the satisfaction questions. In certain embodiments the subject selects a satisfaction rating of “agree” or “strongly agree” to eight or more of the satisfaction questions. In certain embodiments the subject selects a satisfaction rating of “agree” or “strongly agree” to nine or more of the satisfaction questions. In certain embodiments the subject selects a satisfaction rating of “agree” or “strongly agree” to ten or more of the satisfaction questions. In certain embodiments the subject selects a satisfaction rating of “agree” or “strongly agree” to eleven or more of the satisfaction questions. In certain embodiments the subject selects a satisfaction rating of “agree” or “strongly agree” to twelve or more of the satisfaction questions. In certain embodiments the subject selects a satisfaction rating of “agree” or “strongly agree” to thirteen or more of the satisfaction questions. In certain embodiments the subject selects a satisfaction rating of “agree” or “strongly agree” to fourteen or more of the satisfaction questions. In certain embodiments the subject selects a satisfaction rating of “agree” or “strongly agree” to fifteen or more of the satisfaction questions. In certain embodiments the subject selects a satisfaction rating of “agree” or “strongly agree” to sixteen or more of the satisfaction questions. In certain embodiments the subject selects a satisfaction rating of “agree” or “strongly agree” to seventeen or more of the satisfaction questions. In certain embodiments the subject selects a satisfaction rating of “agree” or “strongly agree” to eighteen or more of the satisfaction questions.

Embodiments described herein are directed to methods for developing a remittive effect in a subject with moderate to severe atopic dermatitis comprising administering about 1.0% tapinarof in a topical composition to the affected areas of the subject once a day for an initial period of time until the atopic dermatitis is clear at which time the administration is stopped, and reassessing the subject from about 3 months to about 12 months after administration is stopped to determine if further treatment is required.

Embodiments described herein are directed to methods for treating moderate to severe atopic dermatitis in a subject comprising administering about 1.0% tapinarof in a topical composition to the affected areas of the subject once a day for an initial period of time until the atopic dermatitis is clear at which time the administration is stopped, and reassessing the subject from about 3 months to about 12 months after administration is stopped to determine if further treatment is required.

In embodiments described herein, the topical composition is administered in an initial period of time. In embodiments described herein, the initial period of time is about 8 weeks to about 40 weeks in duration. In certain embodiments, the initial period of time is about 40 weeks.

In embodiments described herein, reassessment of the subject occurs at about 4 months after administration. In certain embodiments, further treatment is initiated after reassessment of the subject. In certain embodiments, the further treatment comprises administering about 1.0% tapinarof in a topical composition to the affected areas of the subject once a day for a subsequent period of time until the atopic dermatitis is clear.

Embodiments described herein are directed to methods for inducing a remittive effect in a subject with atopic dermatitis, wherein the remittive effect can be temporary or permanent.

Embodiments described herein are directed to methods for achieving remission in a subject with atopic dermatitis comprising administering about 1.0% tapinarof in a topical composition to the affected areas of the subject once a day until the subject reaches an IGA score of 0, wherein administration of the about 1.0% tapinarof in a topical composition is stopped. In certain embodiments, the subject reaches an IGA score of 0 at 12 weeks. In certain embodiments, the administration is stopped for about 4 months or indefinitely. In certain embodiments, treatment is reinitiated when the subject reaches a IGA score of ≥2, wherein treatment is continued until the subject achieves a IGA score of 0.

Embodiments described herein are directed to methods for treating a subject with atopic dermatitis who has achieved an IGA score of 0 after about 4 weeks, 8 weeks, or 12 weeks of administration of about 1.0% tapinarof in a topical composition comprising stopping administration of the about 1.0% tapinarof in a topical composition for about 4 months, 5 months, 6 months, 7 months, 8 months, 9 months, 10 months, 11 months, or indefinitely.

Embodiments described herein are directed to methods for achieving remission in a subject with atopic dermatitis comprising administering about 1.0% tapinarof in a topical composition to the affected areas of the subject having a IGA score of ≥1 once a day, wherein administration of the about 1.0% tapinarof in a topical composition is stopped when the subject reaches a IGA score of 0. In certain embodiments, the subject reaches a IGA score of 0 after about 4 weeks, 8 weeks or 12 weeks of treatment. In certain embodiments, once the subject reaches a IGA score of 0 the administration is stopped for about 4 months, 5 months, 6 months, 7 months, 8 months, 9 months, 10 months, 11 months, or indefinitely. In certain embodiments, treatment is reinitiated when the subject reaches a IGA score of ≥2, wherein treatment is continued until the subject achieves a IGA score of 0. Embodiments described herein are directed to methods for maintaining remission of moderate to severe atopic dermatitis in a subject comprising administering about 1.0% tapinarof in a topical composition to the affected areas of the subject once a day for an initial period of time until the atopic dermatitis is clear at which time the administration is stopped and remission is achieved, wherein the atopic dermatitis is clear is defined as one or more symptom is alleviated.

Embodiments described herein are directed to methods for inducing remission of atopic dermatitis in a subject comprising administering about 1.0% tapinarof in a topical composition to the affected areas of the subject once a day for an initial period of time until the atopic dermatitis is clear at which time the administration is stopped and remission has been induced, wherein the atopic dermatitis is clear is defined as one or more symptom is alleviated.

In embodiments described herein, further treatment comprises administering about 1.0% tapinarof in a topical composition to the affected areas of the subject once a day for a subsequent period of time until the atopic dermatitis is treated. In certain embodiments, the subsequent period of time is about 8 weeks to about 16 weeks. In certain embodiments, the subsequent period of time can be up to 52 weeks.

In embodiments described herein, the treating moderate to severe atopic dermatitis in a subject is wherein one or more symptom of atopic dermatitis is improved. In embodiments described herein, the one or more symptom of atopic dermatitis is measured according to an assessment selected from Investigator Global Assessment (IGA) score, daily Itch/Pruritus numeric rating scale, Eczema Area and Severity Index (EASI), total severity score, percent body surface area (BSA) affected, sleep quality, dry/rough skin, red/discolored skin, flaky skin, visual analogue scale (VAS) for sleep, visual analogue scale (VAS) for itch, peak pruritus numerical rating scale (PP-NRS), and patient reported outcomes.

In embodiments described herein, the topically administering of the topical composition includes application to the skin of the body, arms, legs, back, chest, buttocks, neck, scalp, fingernails, or toenails where the atopic dermatitis lesions are present (or “affected area”). The topically administering of the topical composition includes applying enough of the topical composition to completely cover each lesion with a thin layer. In embodiments described herein, administration of the topical composition requires that the subject lightly rub the cream into the skin until it is no longer visible.

In embodiments described herein, the subject has been diagnosed with atopic dermatitis and has had stable disease for at least 6 months, at least 7 months, at least 8 months, at least 9 months, at least 10 months, at least 11 months, or at least 12 months. In embodiments described herein, the subject has been diagnosed with atopic dermatitis and has had stable disease for at least 6 months for ages 6 years old and older. In embodiments described herein, the subject has been diagnosed with atopic dermatitis and has had stable disease for 3 months for ages 2 to 5 years old.

In embodiments described herein, the subject is between the ages of 2 or older. In embodiments described herein, the subject is younger than 18 years of age. In embodiments described herein, the subject is 18 years of age or older. In embodiments described herein, the subject is between the ages of 18 to 65 years old. In embodiments described herein, the subject is between the ages of 18 to 75 years old.

In embodiments described herein, the topical composition is administered once daily for up to 52 weeks. In embodiments described herein, the topical composition is administered once daily for about 52 weeks. In embodiments described herein, the topical composition is administered once daily for up to 48 weeks. In embodiments described herein, the topical composition is administered once daily for about 48 weeks. In embodiments described herein, the topical composition is administered once daily for up to 44 weeks. In embodiments described herein, the topical composition is administered once daily for about 44 weeks. In embodiments described herein, the topical composition is administered once daily for up to 40 weeks. In embodiments described herein, the topical composition is administered once daily for about 40 weeks. In embodiments described herein, the topical composition is administered once daily for up to 36 weeks. In embodiments described herein, the topical composition is administered once daily for about 36 weeks. In embodiments described herein, the topical composition is administered once daily for up to 32 weeks. In embodiments described herein, the topical composition is administered once daily for about 32 weeks. In embodiments described herein, the topical composition is administered once daily for up to 28 weeks. In embodiments described herein, the topical composition is administered once daily for about 28 weeks. In embodiments described herein, the topical composition is administered once daily for up to 24 weeks. In embodiments described herein, the topical composition is administered once daily for about 24 weeks. In embodiments described herein, the topical composition is administered once daily for up to 12 weeks. In embodiments described herein, the topical composition is administered once daily for about 12 weeks. In embodiments described herein, the topical composition is administered once daily for up to 8 weeks. In embodiments described herein, the topical composition is administered once daily for about 8 weeks. In embodiments described herein, the topical composition is administered once daily for up to 6 weeks. In embodiments described herein, the topical composition is administered once daily for about 6 weeks. In embodiments described herein, the topical composition is administered once daily for up to 4 weeks. In embodiments described herein, the topical composition is administered once daily for about 4 weeks. In embodiments described herein, the topical composition is administered once daily until the atopic dermatitis is resolved.

In embodiments described herein, the Investigator's Global Assessment (IGA) is used for assessing the current state/severity of a subject's AD. It uses a static 5-point morphological assessment of overall disease severity, as determined by the investigator, using the clinical characteristics of erythema, infiltration, papulation, oozing, and crusting as guidelines. In certain embodiments, the IGA is made daily, weekly, or monthly and without reference to previous scores. The scoring system ranges from 0 (=Clear) to 4 (=Severe). In embodiments described herein, the subject's Investigator Global Assessment (IGA) score improved by about 1 grade, about 2 grades, about 3 grades, about 4 grades, or about 5 grades. In embodiments described herein, the subject's IGA score improved by about 2 grades. In embodiments described herein, the subject's IGA score improved to a score of about 0 or about 1. In embodiments described herein, the subject's IGA score improved to a score of about 1 or almost clear. In certain embodiments, the subject achieved a score of about 0 or about 1 by week 1, week 2, week 3, week 4, week 5, week 6, week 7, week 8, week 9, week 10, week 11, or week 12. In certain embodiments, the subject achieved a 2-grade improvement by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of IGA score after treatment has ended. In embodiments described herein, the subject has sustained improvement of IGA score about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of IGA score about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In certain embodiments, the duration of treatment success (time off treatment) will correlate with the severity of baseline disease. For example, a subject with a baseline IGA score of 3 may have treatment success for about 4 weeks to about 12 weeks, a subject with a baseline IGA score of 2 may have treatment success for about 8 weeks to about 20 weeks, a subject with a baseline IGA score of 1 may have treatment success for about 12 weeks to about 24 weeks, and a subject with a baseline IGA score of 0 may have treatment success for about 20 weeks to about 52 weeks.

In certain embodiments, the subject achieved a IGA score of 0 or 1 at least once over a 52 week period of time. In certain embodiments, the subject achieved a IGA score of 0 at least once over a 52 week period of time. In certain embodiments, the subject achieved a IGA score of 0 or 1 at least once over a 44 week period of time. In certain embodiments, the subject achieved a IGA score of 0 at least once over a 44 week period of time. In certain embodiments, the subject did not experience worsening during a 52 week period of time.

In embodiments described herein, administration of the topical composition to a subject having moderate to severe atopic dermatitis is effectively treated wherein the subject achieved a “Clear” or “Almost Clear” rating according to the IGA with at least a 2 point improvement.

Pruritus is the most frequent symptom of AD and potentially has the greatest effect on quality of life. In embodiments described herein, the daily Itch/Pruritus numeric rating scale is subject-reported and obtained from the itch item on the Daily Sign and Symptom Severity Diary. In embodiments described herein, the subject's Itch/Pruritus numeric rating scale is improved by about 1 point, about 2 points, about 3 points, about 4 points, or about 5 points. In embodiments described herein, the subject's Itch/Pruritus numeric rating scale is improved by 3 points.

The assessment of the % BSA affected is an estimate of the percentage of total involved skin with atopic dermatitis. The extent of BSA affected by AD is a general indicator of disease severity. In embodiments described herein, one percent body surface area (1% BSA) is the equivalent of the total palmar surface of the palm plus 5 digits. The % BSA affected is calculated using the following regional body areas: Head and neck; Trunk, includes internal axillae and groin; Upper extremities, includes arms, external axillae, and hands; and Lower extremities, includes legs, buttocks, and feet. The % BSA assessment is utilized in the EASI. The % BSA affected by atopic dermatitis is evaluated from 0 to 100%. In embodiments described herein, the subject's percent body surface area (BSA) affected is decreased. In certain embodiments, the subject achieved a decrease in the % BSA affected by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of % BSA affected after treatment has ended. In embodiments described herein, the subject has sustained improvement of percent body surface area (BSA) affected about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of % BSA affected about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the Eczema Area and Severity Index (EASI) is measured using a scoring system for assessing the severity of AD that takes into account the overall severity of erythema, infiltration/papulation, excoriation, and lichenification, as well as the extent of BSA affected with AD. The 4 clinical signs are each graded on a 4-point scale (0 to 3) for each of the 4 specified body regions (head and neck, upper extremities, lower extremities, and trunk). The EASI is a static assessment made without reference to previous scores. In embodiments described herein, the subject's Eczema Area and Severity Index (EASI) is improved by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In embodiments described herein, the subject's Eczema Area and Severity Index (EASI) is improved by greater than or equal to 25%, greater than or equal to 50%, or greater than or equal to 75%. In embodiments described herein, the subject's Eczema Area and Severity Index (EASI) is improved by greater than or equal to 50%. In embodiments described herein, the subject's Eczema Area and Severity Index (EASI) is improved by greater than or equal to 75%. In embodiments described herein, the subject's Eczema Area and Severity Index (EASI) is improved by greater than or equal to 90%. In certain embodiments, the subject achieved a greater than 50% improvement in EASI by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In certain embodiments, the subject achieved a greater than 75% improvement in EASI by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In certain embodiments, the subject achieved a greater than 90% improvement in EASI by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of EASI after treatment has ended. In embodiments described herein, the subject has sustained improvement of EASI about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of EASI about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the total severity score (TSS) is determined by measuring a single target lesion measuring at least 3 cm at baseline and was representative of subject disease, but not located on hands, feet or genitalia. The single target lesion selected to assess efficacy in treating a discrete area rather than an overall average of all areas. For the single target lesion, the severity of erythema, induration/papulation, lichenification, oozing/crusting, and scaling was assessed on a 4-point scale and TSS was calculated. The maximum score was 15, with higher scores indicating more severe disease. In embodiments described herein, the subject's total severity score improved by about 1 point, about 2 points, 3 points, about 4 points, about 5 points, about 6 points, about 7 points, about 8 points, about 9 points, about 10 points, about 11 points, about 12 points, about 13 points, about 14 points, or about 15 points. In certain embodiments, the subject achieved an improvement in total severity score by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of total severity score after treatment has ended. In embodiments described herein, the subject has sustained improvement of total severity score about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of total severity score about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the peak pruritus numerical rating scale (PP-NRS) is used to measure itch intensity. The maximum score was 10, with higher scores indicating worst itch imaginable itch. In embodiments described herein, the subject's total severity score improved by about 1 point, about 2 points, 3 points, about 4 points, about 5 points, about 6 points, about 7 points, about 8 points, about 9 points, or about 10 points. In certain embodiments, the subject achieved an improvement in total severity score by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement of total severity score after treatment has ended. In embodiments described herein, the subject has sustained improvement of total severity score about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of total severity score about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, patient reported outcomes were measured using the Daily Sign and Symptom Severity Diary and the Expanded Patient-Oriented Eczema Measure (POEM).

In embodiments described herein, the self-administered Daily Sign and Symptom Severity Diary assesses the severity of 11 disease-related signs and symptoms (1. skin that is itchy, 2. discolored, 3. bleeding, 4. oozing, 5. cracked, 6. scaly, 7. flaky, 8. dry/rough, 9. painful, 10. burning, and 11. stinging). Response options are on an 11-point numeric rating scale (NRS) and range from 0 (Absent) to 10 (Worst Imaginable). In embodiments described herein, the recall period was the previous 24 hours. In embodiments described herein, the subject's assessment of itchy skin, red/discolored skin, bleeding, weeping or oozing skin, cracked skin, scaly skin, flaky skin, dry or rough skin, painful skin, burning skin, or burning skin, each improved by about 1 point, about 2 points, 3 points, about 4 points, about 5 points, about 6 points, about 7 points, about 8 points, about 9 points, about 10 points, or about 11 points. In certain embodiments, the subject reported an improvement on one or more symptoms assessed by the Daily Sign and Symptom Severity Diary by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement on one or more symptoms assessed by the Daily Sign and Symptom Severity Diary after treatment has ended. In embodiments described herein, the subject has sustained improvement on one or more symptoms assessed by the Daily Sign and Symptom Severity Diary about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of one or more daily activities assessed by the Daily Sign and Symptom Severity Diary about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the subject assessed disease severity using the self-administered Expanded Patient-Oriented Eczema Measure (POEM). In embodiments described herein, the Expanded POEM assessed seven symptoms (1. skin that is itchy, 2. bleeding, 3. weeping/oozing, 4. cracked, 5. flaking, 6. dry/rough, and 7. disturbed sleep) measured using a 5-point scale of frequency of occurrence during the previous week. The 3 questions to assess sleep quality were directed to the frequency of waking at night difficulty falling asleep due to the atopic dermatitis. Individual responses were scored from 0 to 4. Improvement in sleep and improvement in itch were also measured using a visual analogue scale (VAS). In embodiments described herein, the subject's assessment of itchy skin, bleeding, weeping/oozing, cracked skin, flaking skin, dry/rough skin, and disturbed sleep each improved by about 1 point, about 2 points, 3 points, or about 4 points. In embodiments described herein, the subject's assessment of sleep quality is improved. In embodiments described herein, the subject's assessment of disturbed sleep improved. In certain embodiments, the subject reported an improvement on one or more symptoms assessed by POEM by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained improvement on the impact of one or more daily activities assessed by POEM after treatment has ended. In embodiments described herein, the subject has sustained improvement of one or more daily activities assessed by POEM about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening of one or more daily activities assessed by POEM about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the Patient Satisfaction questionnaire includes the questions provided below, wherein the subject selects one of the following: Strongly Agree, Agree, Neutral, Disagree, or Strongly Disagree.
1) I can easily manage the atopic dermatitis with the study drug
2) The time spent applying the study drug every day was acceptable and did not affect my everyday life
3) I am satisfied with how well the study drug worked for my atopic dermatitis
4) I have confidence in the study drug
5) The study drug cleared my skin and kept my atopic dermatitis from coming back
6) If the study drug was available by prescription, I would recommend it to other patients with atopic dermatitis
7) If the study drug was available by a prescription, I would use it again or continue on it
8) The study drug is easy to apply
9) The study drug is not greasy
10) The study drug quickly absorbs into my skin
11) The study drug feels good on my skin
12) I am satisfied with the look and feel of the study drug
13) The study drug is more effective than other topical drugs I have used to treat my atopic dermatitis
14) The study drug is easier to use than other topical drugs I have used to treat my atopic dermatitis
15) I prefer the study drug to other topical drugs I have used to treat my atopic dermatitis
16) The study drug is more effective than systemic drugs I have used to treat my atopic dermatitis
17) The study drug is easier to use than systemic drugs I have used to treat my atopic dermatitis
18) I prefer the study drug to systemic drugs I have used to treat my atopic dermatitis

In embodiments described herein, the one or more symptoms improved by about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after administering the topical composition. In embodiments described herein, the one or more symptoms improved by about 2 weeks of administering the topical composition. In embodiments described herein, the one or more symptoms improved by about 4 weeks of administering the topical composition. In embodiments described herein, the one or more symptoms improved by about 8 weeks of administering the topical composition. In embodiments described herein, the one or more symptoms improved by about 12 weeks of administering the topical composition.

In some embodiments, it was surprisingly found that the topical composition may produce long lasting effects on the skin and may modify the long-term course of moderate to severe atopic dermatitis. Specifically, in certain embodiments, the improvement of the symptoms seen during administration of the topical composition may be maintained long after the final administration of the topical composition. In embodiments described herein, the one or more symptoms remain improved and the remittive effect lasted about 1 week, about 2 weeks, about 3 weeks, about 4 weeks, about 5 weeks, about 6 weeks, about 12 weeks, about 13 weeks, about 14 weeks, about 15 weeks, about 16 weeks, about 17 weeks, about 18 weeks, about 19 weeks, about 20 weeks, or up to 52 weeks after administration of the topical composition has ceased. In embodiments described herein, the one or more symptoms remain improved and the remittive effect lasted about 2 weeks after administration of the topical composition has ceased. In embodiments described herein, the one or more symptoms remain improved and the remittive effect lasted about 3 weeks after administration of the topical composition has ceased. In embodiments described herein, the one or more symptoms remain improved and the remittive effect lasted about 4 weeks after administration of the topical composition has ceased. In embodiments described herein, the one or more symptoms remain improved and the remittive effect lasted about 8 weeks after administration of the topical composition has ceased. In embodiments described herein, the one or more symptoms remain improved and the remittive effect lasted about 12 weeks after administration of the topical composition has ceased. In embodiments described herein, the one or more symptoms remain improved and the remittive effect lasted about 16 weeks after administration of the topical composition has ceased. In embodiments described herein, the one or more symptoms remain improved and the remittive effect lasted about 20 weeks after administration of the topical composition has ceased. In embodiments described herein, the one or more symptoms remain improved and the remittive effect lasted about 24 weeks after administration of the topical composition has ceased. In embodiments described herein, the one or more symptoms remain improved and the remittive effect lasted about 28 weeks after administration of the topical composition has ceased. In embodiments described herein, the one or more symptoms remain improved and the remittive effect lasted about 32 weeks after administration of the topical composition has ceased. In embodiments described herein, the one or more symptoms remain improved and the remittive effect lasted about 36 weeks after administration of the topical composition has ceased. In embodiments described herein, the one or more symptoms remain improved and the remittive effect lasted about 42 weeks after administration of the topical composition has ceased. In embodiments described herein, the one or more symptoms remain improved and the remittive effect lasted about 48 weeks after administration of the topical composition has ceased. In embodiments described herein, the one or more symptoms remain improved and the remittive effect lasted about 52 weeks after administration of the topical composition has ceased.

It was surprisingly found that the topical composition may produce long lasting effects on the skin and may modify the long-term course of moderate to severe atopic dermatitis. Specifically, in certain embodiments, the symptoms do not worsen after the final administration of the topical composition. In embodiments described herein, the one or more symptoms do not worsen about 1 week, about 2 weeks, about 3 weeks, about 4 weeks, about 5 weeks, about 6 weeks, about 12 weeks, about 13 weeks, about 14 weeks, about 15 weeks, about 16 weeks, about 17 weeks, about 18 weeks, about 19 weeks, about 20 weeks, or up to 52 weeks after administration of the topical composition has ceased. In embodiments described herein, the one or more symptoms do not worsen about 2 weeks after administration of the topical composition has ceased. In embodiments described herein, the one or more symptoms do not worsen about 3 weeks after administration of the topical composition has ceased. In embodiments described herein, the one or more symptoms do not worsen about 4 weeks after administration of the topical composition has ceased. In embodiments described herein, the one or more symptoms do not worsen about 8 weeks after administration of the topical composition has ceased. In embodiments described herein, the one or more symptoms do not worsen about 12 weeks after administration of the topical composition has ceased. In embodiments described herein, the one or more symptoms do not worsen about 16 weeks after administration of the topical composition has ceased. In embodiments described herein, the one or more symptoms do not worsen about 20 weeks after administration of the topical composition has ceased. In embodiments described herein, the one or more symptoms do not worsen about 24 weeks after administration of the topical composition has ceased. In embodiments described herein, the one or more symptoms do not worsen about 28 weeks after administration of the topical composition has ceased. In embodiments described herein, the one or more symptoms do not worsen about 32 weeks after administration of the topical composition has ceased. In embodiments described herein, the one or more symptoms do not worsen about 36 weeks after administration of the topical composition has ceased. In embodiments described herein, the one or more symptoms do not worsen about 42 weeks after administration of the topical composition has ceased. In embodiments described herein, the one or more symptoms do not worsen about 48 weeks after administration of the topical composition has ceased. In embodiments described herein, the one or more symptoms do not worsen about 52 weeks after administration of the topical composition has ceased.

In embodiments described herein, the topical composition can be used in a long-term treatment regimen, compared with topical corticosteroids which can only be used for 2-4 weeks. In embodiments described herein, the topical composition can be used for greater than 12 weeks.

Atopic dermatitis is a chronic disease which typically requires daily treatment for the duration of the subject's life. Available treatments, such are corticosteroids, have high side effects and cannot be administered for longer than 2 weeks at a time, after which time a rest phase from treatment must be taken before treatment can resume. Surprisingly, the tapinarof topical composition described herein is safe to use daily for an extended period of time which is greater than 2 weeks. In embodiments described herein, the tapinarof topical composition described herein can be continuously administered to the subject for greater than 2 weeks, greater than 1 month, greater than 2 months, greater than 3 months, greater than 4 months, or longer. Additionally, current treatments are only applied to the areas of the skin where there is an active lesion. Surprisingly, the tapinarof topical composition described herein can be safely applied to all areas of the skin without any limit as to total amount of body surface area receives treatment, for example, the subject can apply to active lesions as well as clear regions. In embodiments described herein, the tapinarof topical composition described herein can be administered to any or all of the following regions: back, elbows, knees, legs, soles of the feet, scalp, face, palms, genitals, or chest.

Embodiments described herein are directed to methods for treating a subject with atopic dermatitis comprising administering about 1.0% tapinarof in a topical composition to the affected areas of the subject once a day for an indefinite amount of time, wherein the subject reaches an IGA score of 1 or improves by at least 1 grade. In embodiments described herein, the subject maintains an improved IGA score but does not reach 0. In embodiments described herein, the subject achieves an improved IGA score of 0 after 4 months of administration of the tapinarof topical composition.

### Methods of Using Topical Compositions to Treat Radiation Dermatitis

Embodiments described herein are directed to methods for developing a remittive effect in a subject with radiation dermatitis comprising administering about 1.0% tapinarof in a topical composition to the affected areas of the subject once a day for an initial period of time until the radiation dermatitis is clear at which time the administration is stopped, and reassessing the subject from about 3 months to about 12 months after administration is stopped to determine if further treatment is required.

Embodiments described herein are directed to methods for treating radiation dermatitis in a subject comprising administering about 1.0% tapinarof in a topical composition to the affected areas of the subject once a day for an initial period of time until the radiation dermatitis is clear at which time the administration is stopped, and reassessing the subject from about 3 months to about 12 months after administration is stopped to determine if further treatment is required.

In embodiments described herein, the topical composition is administered in an initial period of time. In embodiments described herein, the initial period of time is about 16 weeks to about 40 weeks in duration. In certain embodiments, the initial period of time is about 40 weeks.

In embodiments described herein, reassessment of the subject occurs at about 4 months after administration. In certain embodiments, further treatment is initiated after reassessment of the subject. In certain embodiments, the further treatment comprises administering about 1.0% tapinarof in a topical composition to the affected areas of the subject once a day for a subsequent period of time until the radiation dermatitis is clear.

Embodiments described herein are directed to methods for inducing a remittive effect in a subject with radiation dermatitis, wherein the remittive effect can be temporary or permanent.

Embodiments described herein are directed to methods for achieving remission in a subject with radiation dermatitis comprising administering about 1.0% tapinarof in a topical composition to the affected areas of the subject once a day until the subject reaches a National Cancer Institute classification of Grade 1, wherein administration of the about 1.0% tapinarof in a topical composition is stopped. In certain embodiments, the subject reaches a National Cancer Institute classification of Grade 1 at 12 weeks. In certain embodiments, the administration is stopped for about 4 months or indefinitely.

Embodiments described herein are directed to methods for treating a subject with radiation dermatitis who has achieved a National Cancer Institute classification of Grade 1 after 12 weeks of administration of about 1.0% tapinarof in a topical composition comprising stopping administration of the about 1.0% tapinarof in a topical composition for about 4 months.

Embodiments described herein are directed to methods for maintaining remission of radiation dermatitis in a subject comprising administering about 1.0% tapinarof in a topical composition to the affected areas of the subject once a day for an initial period of time until the radiation dermatitis is clear at which time the administration is stopped and remission is achieved, wherein one or more symptom is alleviated.

Embodiments described herein are directed to methods for inducing remission of radiation dermatitis in a subject comprising administering about 1.0% tapinarof in a topical composition to the affected areas of the subject once a day for an initial period of time until the radiation dermatitis is clear at which time the administration is stopped and remission has been induced, wherein one or more symptom is alleviated.

In embodiments described herein, the treating radiation dermatitis in a subject is wherein one or more symptom of radiation dermatitis is improved. In embodiments described herein, the one or more symptom of radiation dermatitis is measured according to an assessment selected from erythema, desquamation, patchy skin, moist desquamation confined to skin folds and creases, moderate swelling, confluent, moist desquamation greater than 1.5 cm diameter, which is not confined to the skin folds, pitting edema (severe swelling), skin necrosis, or ulceration of full-thickness dermis (middle layer of skin).

The National Cancer Institute (USA) has developed 4 criteria for the classification of acute radiation dermatitis: Grade 1—Faint erythema and/or desquamation; Grade 2—Moderate to brisk erythema or patchy, moist desquamation confined to skin folds and creases, and/or Moderate swelling; Grade 3—Confluent, moist desquamation greater than 1.5 cm diameter, which is not confined to the skin folds, and/or Pining edema (severe swelling); and Grade 4—Skin necrosis or ulceration of full-thickness dermis (middle layer of skin).

In embodiments described herein, the topically administering of the topical composition includes application to the skin of the body, arms, legs, back, chest, buttocks, neck, scalp, fingernails, or toenails where the radiation dermatitis lesions are present (or “affected area”). The topically administering of the topical composition includes applying enough of the topical composition to completely cover each lesion with a thin layer. In embodiments described herein, administration of the topical composition requires that the subject lightly rub the cream into the skin until it is no longer visible.

In embodiments described herein, the subject is younger than 18 years of age. In embodiments described herein, the subject is 18 years of age or older. In embodiments described herein, the subject is between the ages of 18 to 65 years old. In embodiments described herein, the subject is between the ages of 18 to 75 years old.

In embodiments described herein, the topical composition is administered once daily for up to 52 weeks. In embodiments described herein, the topical composition is administered once daily for about 52 weeks. In embodiments described herein, the topical composition is administered once daily for up to 48 weeks. In embodiments described herein, the topical composition is administered once daily for about 48 weeks. In embodiments described herein, the topical composition is administered once daily for up to 44 weeks. In embodiments described herein, the topical composition is administered once daily for about 44 weeks. In embodiments described herein, the topical composition is administered once daily for up to 40 weeks. In embodiments described herein, the topical composition is administered once daily for about 40 weeks. In embodiments described herein, the topical composition is administered once daily for up to 36 weeks. In embodiments described herein, the topical composition is administered once daily for about 36 weeks. In embodiments described herein, the topical composition is administered once daily for up to 32 weeks. In embodiments described herein, the topical composition is administered once daily for about 32 weeks. In embodiments described herein, the topical composition is administered once daily for up to 28 weeks. In embodiments described herein, the topical composition is administered once daily for about 28 weeks. In embodiments described herein, the topical composition is administered once daily for up to 24 weeks. In embodiments described herein, the topical composition is administered once daily for about 24 weeks. In embodiments described herein, the topical composition is administered once daily for up to 12 weeks. In embodiments described herein, the topical composition is administered once daily for about 12 weeks. In embodiments described herein, the topical composition is administered once daily for up to 8 weeks. In embodiments described herein, the topical composition is administered once daily for about 8 weeks. In embodiments described herein, the topical composition is administered once daily for up to 6 weeks. In embodiments described herein, the topical composition is administered once daily for about 6 weeks. In embodiments described herein, the topical composition is administered once daily for up to 4 weeks. In embodiments described herein, the topical composition is administered once daily for about 4 weeks. In embodiments described herein, the topical composition is administered once daily until the radiation dermatitis is resolved.

In certain embodiments, the subject achieved a classification of Grade 1 by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, or week 24. In embodiments described herein, the subject has sustained classification of Grade 1 after treatment has ended. In embodiments described herein, the subject has sustained classification of Grade 1 for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In embodiments described herein, the subject does not experience worsening to Grades 2-4 for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

In embodiments described herein, the one or more symptoms improved by about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after administering the topical composition. In embodiments described herein, the one or more symptoms improved by about 2 weeks of administering the topical composition. In embodiments described herein, the one or more symptoms improved by about 4 weeks of administering the topical composition. In embodiments described herein, the one or more symptoms improved by about 8 weeks of administering the topical composition. In embodiments described herein, the one or more symptoms improved by about 12 weeks of administering the topical composition.

In some embodiments, it was surprisingly found that the topical composition may produce long lasting effects on the skin and may modify the long-term course of radiation dermatitis. Specifically, in certain embodiments, the improvement of the symptoms seen during administration of the topical composition may be maintained long after the final administration of the topical composition. In embodiments described herein, the one or more symptoms remain improved and the remittive effect lasted about 1 week, about 2 weeks, about 3 weeks, about 4 weeks, about 5 weeks, about 6 weeks, about 12 weeks, about 13 weeks, about 14 weeks, about 15 weeks, about 16 weeks, about 17 weeks, about 18 weeks, about 19 weeks, about 20 weeks, or up to 52 weeks after administration of the topical composition has ceased. In embodiments described herein, the one or more symptoms remain improved and the remittive effect lasted about 2 weeks after administration of the topical composition has ceased. In embodiments described herein, the one or more symptoms remain improved and the remittive effect lasted about 3 weeks after administration of the topical composition has ceased. In embodiments described herein, the one or more symptoms remain improved and the remittive effect lasted about 4 weeks after administration of the topical composition has ceased. In embodiments described herein, the one or more symptoms remain improved and the remittive effect lasted about 8 weeks after administration of the topical composition has ceased. In embodiments described herein, the one or more symptoms remain improved and the remittive effect lasted about 12 weeks after administration of the topical composition has ceased. In embodiments described herein, the one or more symptoms remain improved and the remittive effect lasted about 16 weeks after administration of the topical composition has ceased. In embodiments described herein, the one or more symptoms remain improved and the remittive effect lasted about 20 weeks after administration of the topical composition has ceased. In embodiments described herein, the one or more symptoms remain improved and the remittive effect lasted about 24 weeks after administration of the topical composition has ceased. In embodiments described herein, the one or more symptoms remain improved and the remittive effect lasted about 28 weeks after administration of the topical composition has ceased.

It was surprisingly found that the topical composition may produce long lasting effects on the skin and may modify the long-term course of radiation dermatitis. Specifically, in certain embodiments, the symptoms do not worsen after the final administration of the topical composition. In embodiments described herein, the one or more symptoms do not worsen about 1 week, about 2 weeks, about 3 weeks, about 4 weeks, about 5 weeks, about 6 weeks, about 12 weeks, about 13 weeks, about 14 weeks, about 15 weeks, about 16 weeks, about 17 weeks, about 18 weeks, about 19 weeks, about 20 weeks, or up to 52 weeks after administration of the topical composition has ceased. In embodiments described herein, the one or more symptoms do not worsen about 2 weeks after administration of the topical composition has ceased. In embodiments described herein, the one or more symptoms do not worsen about 3 weeks after administration of the topical composition has ceased. In embodiments described herein, the one or more symptoms do not worsen about 4 weeks after administration of the topical composition has ceased. In embodiments described herein, the one or more symptoms do not worsen about 8 weeks after administration of the topical composition has ceased. In embodiments described herein, the one or more symptoms do not worsen about 12 weeks after administration of the topical composition has ceased. In embodiments described herein, the one or more symptoms do not worsen about 16 weeks after administration of the topical composition has ceased. In embodiments described herein, the one or more symptoms do not worsen about 20 weeks after administration of the topical composition has ceased. In embodiments described herein, the one or more symptoms do not worsen about 24 weeks after administration of the topical composition has ceased. In embodiments described herein, the one or more symptoms do not worsen about 28 weeks after administration of the topical composition has ceased.

In embodiments described herein, the topical composition can be used in a long-term treatment regimen, compared with topical corticosteroids which can only be used for 2-4 weeks. In embodiments described herein, the topical composition can be used for greater than 12 weeks.

Radiation dermatitis typically requires daily treatment but available treatments, such are corticosteroids, have high side effects and cannot be administered for longer than 2 weeks at a time, after which time a rest phase from treatment must be taken before treatment can resume. Surprisingly, the tapinarof topical composition described herein is safe to use daily for an extended period of time which is greater than 2 weeks. In embodiments described herein, the tapinarof topical composition described herein can be continuously administered to the subject for greater than 2 weeks, greater than 1 month, greater than 2 months, greater than 3 months, greater than 4 months, or longer. Additionally, current treatments are only applied to the areas of the skin where there is an active lesion. Surprisingly, the tapinarof topical composition described herein can be safely applied to all areas of the skin without any limit as to total amount of body surface area receives treatment, for example, the subject can apply to active lesions as well as clear regions. In embodiments described herein, the tapinarof topical composition described herein can be administered to any or all of the following regions: back, elbows, knees, legs, soles of the feet, scalp, face, palms, genitals, or chest.

Embodiments described herein are directed to methods for treating a subject with radiation dermatitis comprising administering about 1.0% tapinarof in a topical composition to the affected areas of the subject once a day for an indefinite amount of time, wherein the subject reaches a National Cancer Institute classification of Grade 2 or improves by at least 1 grade. In embodiments described herein, the subject maintains an improved classification but does not reach Grade 1. In embodiments described herein, the subject achieves an improved classification of Grade 1 after 4 months of administration of the tapinarof topical composition.

### Additional Methods of Use

In embodiments described herein, the method of treating a skin condition comprises topically administering the tapinarof composition described herein, wherein the tapinarof has an effect on antigen presenting cell (APC) development, maturation, polarization, activation, or blockade of APC:T-cell interactions.

In embodiments described herein, the method of treating a skin condition comprises topically administering the tapinarof composition described herein, wherein the tapinarof induces T cell polarization, T cell tolerance, T cell anergy, or T cell exhaustion.

In embodiments described herein, the method of treating a skin condition comprises topically administering the tapinarof composition described herein, wherein the tapinarof blocks regulation of T cell differentiation, polarization of T cells, or generation of resident memory T cells.

In embodiments described herein, the tapinarof topical composition is used to treat skin conditions where APC and T cells play a role in disease pathogenesis. In certain embodiments, the disease is psoriasis, atopic dermatitis, or radiation dermatitis.

In embodiments described herein, the method of treating a skin condition comprises topically administering the tapinarof composition described herein, wherein the tapinarof induces disease remittance via alteration of the APC:T cell balance.

In embodiments described herein, the method of treating a skin condition comprises topically administering the tapinarof composition described herein, wherein the tapinarof induces disease remittance via alteration of the TH17:Treg cell balance.

In embodiments described herein, the method of treating a skin condition comprises topically administering the tapinarof composition described herein, wherein the tapinarof induces disease remittance via alteration of the TH1:Treg cell balance.

In embodiments described herein, the method of treating a skin condition comprises topically administering the tapinarof composition described herein, wherein the tapinarof induces disease remittance via alteration of the Th2:Treg cell balance.

In embodiments described herein, the method of treating a skin condition comprises topically administering the tapinarof composition described herein, wherein the tapinarof induces disease remittance via alteration of the Teffector:Treg cell balance.

In embodiments described herein, the method of treating a skin condition comprises topically administering the tapinarof composition described herein, wherein the tapinarof results in disease remittance through AhR modulation of APC:T cell interactions.

In embodiments described herein, the method of treating a skin condition comprises topically administering the tapinarof composition described herein, wherein the tapinarof suppresses or alters the proliferation (growth) of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the development of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the differentiation of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the maturation of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the activation of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the biologic behavior of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the migration and/or chemotaxis of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the polarization of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the gene expression of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the protein expression of and/or the post translational modification of proteins expressed by one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof suppresses or alters the interaction of antigen presenting cells with one or more cell type selected from the group consisting of T-lymphocytes, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof suppresses or alters the interaction of T cells (CD3) with one or more cell type selected from the group consisting of antigen presenting cells, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof suppresses or alters the interaction of T cells (CD4) with one or more cell type selected from the group consisting of antigen presenting cells, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof suppresses or alters the interaction of T cells (CD8) with one or more cell type selected from the group consisting of antigen presenting cells, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof suppresses or alters the interaction of T-helper-17 T cells (TH-17) with one or more cell type selected from the group consisting of antigen presenting cells, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof suppresses or alters the interaction of T-helper-2 T cells (TH-2) with one or more cell type selected from the group consisting of antigen presenting cells, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof suppresses or alters the interaction of T-helper-1 T cells (TH-1) with one or more cell type selected from the group consisting of antigen presenting cells, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof suppresses or alters the interaction of memory resident T cells (Trm) with one or more cell type selected from the group consisting of antigen presenting cells, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof suppresses or alters the interaction of regulatory T cells (Treg) with one or more cell type selected from the group consisting of antigen presenting cells, macrophages, keratinocytes, vascular endothelial cells, mast cells, neutrophils, eosinophils, basophils, and B-lymphocytes.

In embodiments described herein, the method of treating a skin condition comprises topically administering the tapinarof composition described herein, wherein the tapinarof induces or alters the tolerance/anergy of one or more cell type selected from the group consisting T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

In embodiments described herein, the method of treating a skin condition comprises topically administering the tapinarof composition described herein, wherein the tapinarof alters the exhaustion of one or more cell type selected from the group consisting of antigen presenting cells, T cells (CD3), T-helper cells (CD4), T-suppressor cells (CD8), T-helper-17 T-cells (TH-17), T-helper-2 T-cells (TH-2), T-helper-1 T-cells (TH-1), memory resident T-cells (Trm), and regulatory T-cells (Treg).

### Additional Embodiments

Embodiment 1: A method for treating moderate to severe plaque psoriasis in a subject comprising administering about 1.0% tapinarof in a topical composition to the affected areas of the subject once a day for an initial period of time until the plaque psoriasis is clear at which time the administration is stopped, and reassessing the subject from about 3 months to about 12 months after administration is stopped to determine if further treatment is required.

Embodiment 2: The method of Embodiment 1, wherein the initial period of time is about 16 weeks to about 40 weeks.

Embodiment 3: The method of Embodiment 1, wherein plaque psoriasis is clear when the Physician Global Assessment (PGA) score is 0 or 1.

Embodiment 4: The method of Embodiment 1, wherein further treatment is required if the PGA score is greater than or equal to 2.

Embodiment 5: The method of Embodiment 4, wherein further treatment comprises administering about 1.0% tapinarof in a topical composition to the affected areas of the subject once a day for a subsequent period of time until the plaque psoriasis is clear.

Embodiment 6: The method of Embodiment 5, wherein the subsequent period of time is about 8 weeks to about 16 weeks.

Embodiment 7: The method of Embodiment 2, wherein the initial period of time is about 40 weeks.

Embodiment 8: The method of Embodiment 7, wherein reassessment of the subject occurs at about 4 months after administration.

Embodiment 9: The method of Embodiment 8, wherein further treatment is initiated.

Embodiment 10: The method of Embodiment 9, wherein further treatment comprises administering about 1.0% tapinarof in a topical composition to the affected areas of the subject once a day for a subsequent period of time until the plaque psoriasis is clear.

Embodiment 11: A method for treating moderate to severe atopic dermatitis in a subject comprising administering about 1.0% tapinarof in a topical composition to the affected areas of the subject once a day for an initial period of time until the atopic dermatitis is clear at which time the administration is stopped, and reassessing the subject from about 3 months to about 12 months after administration is stopped to determine if further treatment is required.

Embodiment 12: The method of Embodiment 11, wherein the initial period of time is about 16 weeks to about 40 weeks.

Embodiment 13: The method of Embodiment 11, wherein atopic dermatitis is clear when the Investigator Global Assessment (IGA) score is 0 or 1.

Embodiment 14: The method of Embodiment 11, wherein further treatment is required if the IGA score is greater than or equal to 2.

Embodiment 15: The method of Embodiment 14, wherein further treatment comprises administering about 1.0% tapinarof in a topical composition to the affected areas of the subject once a day for a subsequent period of time until the atopic dermatitis is clear.

Embodiment 16: The method of Embodiment 15, wherein the subsequent period of time is about 8 weeks to about 16 weeks.

Embodiment 17: The method of Embodiment 12, wherein the initial period of time is about 40 weeks.

Embodiment 18: The method of Embodiment 17, wherein reassessment of the subject occurs at about 4 months after administration.

Embodiment 19: The method of Embodiment 18, wherein further treatment is initiated.

Embodiment 20: The method of Embodiment 19, wherein further treatment comprises administering about 1.0% tapinarof in a topical composition to the affected areas of the subject once a day for a subsequent period of time until the atopic dermatitis is clear.

Embodiment 21: A method for treating radiation dermatitis in a subject comprising administering about 1.0% tapinarof in a topical composition to the affected areas of the subject once a day for an initial period of time until the radiation dermatitis is clear at which time the administration is stopped, and reassessing the subject from about 3 months to about 12 months after administration is stopped to determine if further treatment is required.

Embodiment 22: The method of Embodiment 21, wherein the initial period of time is about 16 weeks to about 40 weeks.

Embodiment 23: The method of Embodiment 21, wherein radiation dermatitis is clear when the Grade Classification score is 1.

Embodiment 24: The method of Embodiment 21, wherein further treatment is required if the Grade Classification score is greater than or equal to 2.

Embodiment 25: The method of Embodiment 24, wherein further treatment comprises administering about 1.0% tapinarof in a topical composition to the affected areas of the subject once a day for a subsequent period of time until the radiation dermatitis is clear.

Embodiment 26: The method of Embodiment 25, wherein the subsequent period of time is about 8 weeks to about 16 weeks.

Embodiment 27: The method of Embodiment 22, wherein the initial period of time is about 40 weeks.

Embodiment 28: The method of Embodiment 27, wherein reassessment of the subject occurs at about 4 months after administration.

Embodiment 29: The method of Embodiment 28, wherein further treatment is initiated.

Embodiment 30: The method of Embodiment 29, wherein further treatment comprises administering about 1.0% tapinarof in a topical composition to the affected areas of the subject once a day for a subsequent period of time until the radiation dermatitis is clear.

Embodiment 31: A method for achieving remission in a subject with moderate to severe plaque psoriasis comprising administering about 1.0% tapinarof in a topical composition to the affected areas of the subject once a day until the subject reaches a PGA score of 0, wherein administration of the about 1.0% tapinarof in a topical composition is stopped.

Embodiment 32: The method of Embodiment 31, wherein the subject reaches a PGA score of 0 at about 4 weeks, 8 weeks, or 12 weeks.

Embodiment 33: The method of Embodiment 31, wherein the administration is stopped for about 4 months or indefinitely.

Embodiment 34: A method for treating a subject with moderate to severe plaque psoriasis who has achieved a PGA score of 0 after 12 weeks of administration of about 1.0% tapinarof in a topical composition comprising stopping administration of the about 1.0% tapinarof in a topical composition for about 4 months.

Embodiment 35: A method for treating a subject with moderate to severe plaque psoriasis comprising administering about 1.0% tapinarof in a topical composition to the affected areas of the subject once a day for an indefinite amount of time, wherein the subject reaches a PGA score of 1 or improves by at least 1 grade.

Embodiment 36: The method of Embodiment 35, wherein the subject maintains an improved PGA score but does not reach 0.

Embodiment 37: The method of Embodiment 35, wherein the subject achieves an improved PGA score of 0 after 4 months of administration of the tapinarof topical composition.

Embodiment 38: A method for achieving remission in a subject with atopic dermatitis comprising administering about 1.0% tapinarof in a topical composition to the affected areas of the subject once a day until the subject reaches an IGA score of 0, wherein administration of the about 1.0% tapinarof in a topical composition is stopped.

Embodiment 39: The method of Embodiment 38, wherein the subject reaches a IGA score of 0 at about 4 weeks, 8 weeks, or 12 weeks.

Embodiment 40: The method of Embodiment 38, wherein the administration is stopped for about 4 months or indefinitely.

Embodiment 41: A method for treating a subject with atopic dermatitis who has achieved a PGA score of 0 after 12 weeks of administration of about 1.0% tapinarof in a topical composition comprising stopping administration of the about 1.0% tapinarof in a topical composition for about 4 months.

Embodiment 42: A method for treating a subject with atopic dermatitis comprising administering about 1.0% tapinarof in a topical composition to the affected areas of the subject once a day for an indefinite amount of time, wherein the subject reaches an IGA score of 1 or improves by at least 1 grade.

Embodiment 43: The method of Embodiment 42, wherein the subject maintains an improved IGA score but does not reach 0.

Embodiment 44: The method of Embodiment 42, wherein the subject achieves an improved IGA score of 0 after 4 months of administration of the tapinarof topical composition.

Embodiment 45: A method for achieving remission in a subject with radiation dermatitis comprising administering about 1.0% tapinarof in a topical composition to the affected areas of the subject once a day until the subject reaches a National Cancer Institute classification of Grade 1, wherein administration of the about 1.0% tapinarof in a topical composition is stopped.

Embodiment 46: The method of Embodiment 45, wherein the subject reaches a National Cancer Institute classification of Grade 1 at 12 weeks.

Embodiment 47: The method of Embodiment 45, wherein the administration is stopped for an indefinite amount of time.

Embodiment 48: A method for treating a subject with atopic dermatitis who has achieved a National Cancer Institute classification of Grade 1 after 12 weeks of administration of about 1.0% tapinarof in a topical composition comprising stopping administration of the about 1.0% tapinarof in a topical composition for about 4 months.

Embodiment 49: A method for treating a subject with atopic dermatitis comprising administering about 1.0% tapinarof in a topical composition to the affected areas of the subject once a day for about 4 months or indefinitely, wherein the subject reaches a National Cancer Institute classification of Grade 2 or improves by at least 1 grade.

Embodiment 50: The method of Embodiment 49, wherein the subject maintains an improved classification but does not reach Grade 1.

Embodiment 51: The method of Embodiment 49, wherein the subject achieves an improved National Cancer Institute classification of Grade 1 after 4 months of administration of the tapinarof topical composition.

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### EXAMPLES

### Example 1: A Long-Term, Open-Label, Extension Study to Evaluate the Safety and Efficacy of Tapinarof Cream, 1% for the Treatment of Plaque Psoriasis in Adults

Background

Psoriasis is a common, chronic relapsing inflammatory skin disease with recurrent episodes of prominently erythematous and scaly patches. Approximately 2% to 3% of the global population is affected by psoriasis; those affected are predominantly adults, who are most often diagnosed between the ages of 18 to 35 years.

Up to 80% of patients have mild to moderate plaque type psoriasis, which is generally managed with topical treatments such as corticosteroids and/or vitamin D analogs. However, there remains a need for an efficacious and safe topical treatment that permits application to a large body surface area (BSA) for an unrestricted treatment duration.

Tapinarof cream, 1% is an oil in water emulsion intended for topical application to psoriatic skin lesions. The drug likely mediates its effects via the aryl hydrocarbon receptor (AhR) agonist and nuclear factor erythroid 2 related factor 2 because the pattern of proinflammatory mediators inhibited by tapinarof is different from that of corticosteroids, vitamin D analogs, and other immunosuppressive agents commonly used to treat psoriasis. The profile of biological responses elicited by tapinarof most closely matches that of the dual activation properties of coal tar, a common nonprescription treatment for psoriasis. Existing data identify tapinarof as a nonsteroidal therapeutic AhR modulating agent, which has a unique mechanism of action compared with existing therapies. Additional details, including evaluation of tapinarof cream, 1% in nonclinical and clinical trials, can be found in the study protocol.

Study Rationale

This 40 week, Phase 3, open label extension study was conducted as part of a clinical development program to evaluate the long term safety and continued efficacy of tapinarof cream, 1% for the topical treatment of plaque psoriasis in adults. Subjects in this study were enrolled from those subjects who completed 1 of 2 Phase 3 pivotal efficacy and safety studies (Study DMVT 505 3001 or Study DMVT 505 3002) and met the predefined criteria to enroll into this extension study. The results of this extension study are intended to support product registration in the United States.

Rationale for Study Design and Dose

The once daily topical application of tapinarof cream, 1% administered in the pivotal Phase 3 studies was continued in this open label extension study.

Tapinarof cream, 1% is intended for long term use in non-life threatening inflammatory dermatologic conditions. Therefore, the 40 week duration of long term use of tapinarof cream, 1% in this extension study, in addition to the treatment received during the double blinded (DB), pivotal Phase 3 study, was expected to be an adequate duration to assess safety and efficacy of repeated treatment courses of tapinarof cream, 1% as recommended in the ICH E1 guideline on the extent of population exposure to assess clinical safety for drugs intended for long term treatment of non-life threatening conditions. The ICH E1 guideline recommends subject be treated with the intended clinical dose and followed for adverse events (AEs) for at least 1 year for a minimum of 100 subjects and for at least 6 months for a minimum of 300 subjects. This interim analysis includes 235 subjects exposed to tapinarof cream, 1% for 1 year and 450 subjects exposed to tapinarof cream, 1% for 6 months.

Objective: To evaluate the safety and tolerability of tapinarof cream, 1% in adults with plaque psoriasis. Endpoints: Incidence, frequency, and nature of AEs, adverse events of special interest (AESIs), and serious adverse events (SAEs), Change over time in clinical laboratory tests and frequency of clinically significant abnormal test results, Change over time in vital signs and frequency of clinically significant abnormal results, Local Tolerability Scale (LTS) scores by severity summarized by visit.

Objective: To describe the efficacy of tapinarof cream, 1% over an extended period of time in adults with plaque psoriasis, including duration of treatment success, durability of response and duration of remittive effect. Endpoints: Proportion of subjects who experienced a Physician Global Assessment (PGA)≥2 at least 1 time in the study and the median time from Visit 1 date to first worsening (PGA≥2) for subjects entering the study with a PGA score of clear (0), Proportion of subjects who achieved a PGA score of 0 at least 1 time in the study and the median time from Visit 1 date to first achieving a PGA score of 0 for subjects entering the study with a PGA score ≥1, Duration of each treatment episode defined as time from each PGA≥2 (or PGA≥1 for the first episode) to each subsequent treatment success (PGA score of 0), Duration of each treatment success (PGA score of 0) to each subsequent worsening (PGA≥2), Proportion of subjects who never achieved a PGA≥2 throughout the study, Proportion of subjects who never achieved a PGA score of 0 or 1 throughout the study, Proportion of subjects who never achieved a PGA score of 0 throughout the study, PGA scores by visit (Observed Cases [OC] and Last Observed Carried Forward [LOCF]), Change and percent change from baseline in percent of body surface area (% BSA) affected by visit (OC and LOCF), Change and percent change in Psoriasis Area and Severity Index (PASI) score by visit (OC and LOCF).

Objective: To describe the effect of tapinarof cream, 1% on psoriasis symptom severity and the associated impact on daily activities and attitudes in adults with plaque psoriasis. Endpoints: Change in disease impact on daily activities, as measured by the Dermatology Life Quality Index (DLQI) total and individual dimension scores.

Complete disease clearance is defined as the proportion of patients achieving PGA of 0 (clear).

Remittive effect is defined as the duration of efficacy maintenance, which is time at which patient has a PGA score of 0 (clear) or 1 (almost clear) while off therapy after having achieved complete disease clearance (PGA=0).

Response is defined as the proportion of patients who entered the trial with a PGA≥2 and achieved a PGA of 0 (clear) or 1 (almost clear) at least once during trial.

Durability of response (absence of tachyphylaxis) is defined as the maintenance of efficacy while on treatment, defined as the proportion of patients who achieved a PGA score of 0 or 1 at least once during the trial and trends in PASI score and percentage of body surface area (% BSA) affected over time.

Tolerability is defined as the local tolerability using a patient-reported 5-point scale for burning/stinging and itching, and an investigator-assessed 5-point scale for dryness, erythema, and peeling.

Study Design

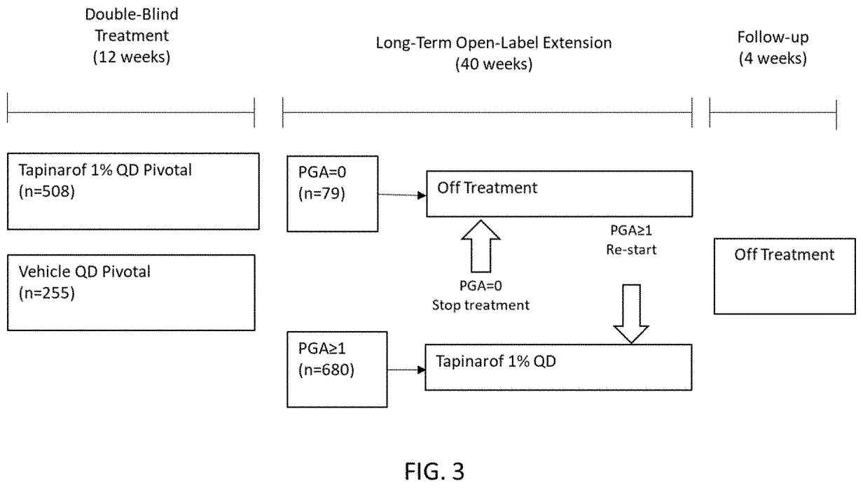

This was a long term, open label, multicenter study to evaluate the safety and efficacy of topical tapinarof cream, 1% in adults with plaque psoriasis. Subjects in this extension study completed treatment with tapinarof cream, 1% or vehicle cream in 1 of 2 DB, randomized, pivotal Phase 3 efficacy and safety studies (Study DMVT 505 3001 or Study DMVT 505 3002). This extension study consisted of up to 40 weeks of treatment and a 4 week, safety follow up period. See FIG. 3.

At the completion of the Week 12 visit of the DB, pivotal Phase 3 study, eligibility of the subjects opting to enroll in this extension study was confirmed. Study visits during the treatment period for all subjects occurred every 4 weeks (±3 days). A phone call was performed at Week 2 (Day 15). Unscheduled visits may have occurred, as needed. Subjects who withdrew from this study before Week 40 returned to the study center for an Early Termination visit. The total duration of this extension study participation was 44 weeks. Subjects in this study were treated as follows based on their PGA score from the Week 12 visit in the DB, pivotal Phase 3 study (Study DMVT 505 3001 or Study DMVT 505 3002).

Subjects entering with a PGA≥1 received treatment with tapinarof cream, 1% until they achieve a PGA=0, at which time treatment was discontinued and subjects were monitored for durability of response (remittive response). If/when disease worsening occurred, as evidenced by a PGA≥2, treatment was re initiated and continued until a PGA=0 was observed.

Subjects entering with a PGA=0 had treatment discontinued and were monitored for remittive response. If/when disease worsening occurred, as evidenced by a PGA≥2, treatment was re initiated and continued until a PGA=0 was observed.

This treatment and re treatment pattern of use was continued until the end of the study (i.e., subjects may have received study drug up until the Week 40 visit.

Study drug (tapinarof cream, 1%) was dispensed, and subjects were instructed on how to apply it. Study drug was dispensed to subjects during the study visits and was to be administered at home between study visits as instructed by site personnel. Subjects were instructed to apply study drug once daily to all affected areas, including newly appearing lesions and lesions or affected areas that improved during the study. Subjects applied sufficient study drug to cover completely each lesion with a thin layer and recorded the time of study drug application in a daily diary provided by the study site. (Note that subjects were allowed, but not required, to treat fingernails, toenails, palms, soles, and scalp lesions with study drug; however, efficacy analyses did not include assessment of improvement of psoriasis in these areas.) Subjects were instructed to maintain the approximate dosing time chosen at the beginning of the study for their full study participation. Study drug application instructions were reviewed at subsequent study visits. During study visits, subjects applied the daily dose of study drug while at the site under the supervision of site personnel, after efficacy and safety assessments had been completed. The time of the dose application and assessments depended on the time of the study visit (either morning or afternoon visit). Therefore, the timing of the study visit may have led to a change in the subject's chosen dosing time; if this occurred, it was not considered a protocol deviation. The subject should have resumed their chosen dosing time the day following any such clinic visit application.

Rescue medications may have been initiated with consultation of the Medical Monitor.

Safety assessments included AEs, AESIs, SAEs, local (application site) tolerability, clinical laboratory tests, vital signs, physical examinations, and Investigator assessed LTS. Efficacy assessments included a 5 point static PGA (0 [clear], 1 [almost clear], 2 [mild], 3 [moderate], 4 [severe]), % BSA affected, the PASI, and subject reported DLQI.

Inclusion Criteria

Each subject had to meet all of the following criteria to be eligible to participate in the study:

Completed the 12 week treatment period in 1 of the 2 pivotal Phase 3 studies (Study DMVT 505 3001 or Study DMVT 505 3002)

Male or female

Female subjects of childbearing potential and male subjects who were engaging in sexual activity that could have led to pregnancy should have used one of the following acceptable birth control methods while on study and for 4 weeks after the last exposure to study drug. Acceptable contraception methods were:

Female subject or male subject's female partner was surgically sterile (bilateral tubal ligation, hysterectomy, bilateral oophorectomy) for a minimum of 3 months prior to the first dose of study drug

Female subject or male subject's female partner had an intrauterine device in place for at least 3 months prior to the first dose of study drug

Double barrier methods (e.g., condom plus diaphragm, condom or diaphragm plus spermicide) starting at least 14 days prior to the first dose of study drug

Subjects who claimed abstinence as their method of contraception were allowed, provided they agreed to use a double barrier method (e.g., condom plus diaphragm, condom or diaphragm plus spermicide) should they have become sexually active from Screening to 1 month after the last dose of study drug

Surgical sterilization of male subject or female subject's male partner (vasectomy) a minimum of 3 months prior to first dose of study drug

Female subject or male subject's female partner was taking hormonal contraceptives starting at least 3 months prior to the first dose of study drug. If hormonal contraceptives were started <3 months prior to the first dose of study drug, subjects must have agreed to use a double barrier method (e.g., condom plus diaphragm, condom or diaphragm plus spermicide) from Screening through 3 months after the initiation of hormonal contraceptives.

These allowed methods of contraception are only effective when used consistently, correctly and in accordance with the product label. The Investigator was responsible for ensuring that subjects understood how to properly use these methods of contraception.

Non childbearing potential was defined as premenarchal or premenopausal females with a documented bilateral tubal ligation, bilateral oophorectomy (removal of the ovaries) or hysterectomy, or hysteroscopic sterilization; or postmenopausal females defined as a cessation of menses for at least 12 months without an alternative medical cause. In questionable cases, a blood sample with simultaneous follicle stimulating hormone >40 mIU/mL was confirmatory. Documented verbal history from the subject was acceptable.

Female subjects of childbearing potential must have had a negative urine pregnancy test at Baseline (Day 1).

Capable of giving signed informed consent, as applicable, which included compliance with the requirements and restrictions listed in the ICF; written informed consent must have been obtained prior to any study related procedures.

Exclusion Criteria

A subject who met any of the following criteria was excluded and considered ineligible for participation in this extension study:

Used a prohibited concomitant product or procedure to treat psoriasis during the pivotal study

Had an SAE that was potentially related to treatment or experienced an AE that led to permanent discontinuation of treatment in the pivotal study

History of or ongoing serious illness or medical, physical, or psychiatric condition(s) that, in the Investigator's opinion, may have interfered with the subject's completion of the study

Known Hypersensitivity to Tapinarof or Excipients

Investigational Product: tapinarof cream, 1%. Excipients in tapinarof cream are propylene glycol, diethylene glycol monoethyl ether, polysorbate 80, medium chain triglycerides, emulsifying wax nonionic, polyoxyl stearyl ether 2, polyoxyl stearyl ether 20, benzoic acid, butylated hydroxytoluene, purified water, sodium citrate, citric acid monohydrate, and edetate disodium.

Treatment by Physician Global Assessment Score

Subjects entering with a PGA≥1 received treatment with tapinarof cream, 1% until they achieved a PGA=0. If/when disease worsening occurred, as evidenced by a PGA≥2, treatment was re initiated and continued until a PGA=0 was observed.

Subjects entering with a PGA=0 had treatment discontinued and were monitored for durability of response (remittive response). If/when disease worsening occurred, as evidenced by a PGA≥2, treatment was re initiated and continued until a PGA=0 was observed.

This treatment and re treatment pattern of use continued until the end of the study (i.e., subjects may have received study drug up until the Week 40 visit). Subjects who experienced suspected disease worsening between scheduled study visits may have contacted the study site to arrange an unscheduled visit. Upon confirmation of disease worsening, subjects would have restarted treatment with study drug.

Disease Worsening (Physician Global Assessment Score ≥2) During the Study

If disease worsening was suspected between scheduled study visits, an unscheduled visit may have been performed.

If disease worsening (i.e., PGA≥2) was not confirmed, the subject continued to be assessed at routine, scheduled visits (see Table 2).

For each confirmed episode of disease worsening (i.e., PGA≥2) during the study (at either a scheduled or unscheduled visit), a treatment course of study drug (once daily) was initiated and continued until the subject achieved a PGA=0, as assessed at scheduled visits, approximately every 4 weeks.

Subjects subsequently achieving a PGA=0 stopped study drug and then were assessed at routine, scheduled visits.

Subjects who then experience suspected disease worsening between scheduled study visits may have contacted the study site to arrange an unscheduled visit.

If disease worsening was not confirmed, the subject continued to be assessed at routine, scheduled visits.

If disease worsening was confirmed (i.e., PGA≥2) at the unscheduled visit, subjects initiated another treatment course of study drug (once daily) until a PGA=0 was achieved at which time the subject stopped study drug and continued to be assessed at routine, scheduled visits.

This treatment and re treatment pattern of use continued until the end of the study.

Application of Study Drug

Study drug was dispensed to subjects requiring treatment at the study visits specified in the Schedule of Assessments.

Subjects took the tubes home and self-administered study drug (or had caregiver apply if necessary), except on clinic visit days (when study drug was applied under supervision at the site), to affected areas, once daily.

Subjects were instructed to apply study drug as follows:

For subjects with a PGA>0, once daily application to affected areas including those areas treated in the DB, pivotal Phase 3 study; subjects were to choose the application time they preferred and applied the study drug at that time each day of study participation.

Subjects with a PGA=0 did not receive treatment but if they worsen and experienced a PGA≥2, study drug was applied once daily to newly affected areas only.

If a subject missed a daily dose, it was recorded as a protocol deviation. The subject was to continue dosing the next day and was not to apply more than once daily to make up for the missed dose on the previous day.

If the chosen application time was in the evening, the dose was applied at least 30 minutes prior to bedtime.

Study drug was to be applied to dry, clean skin.

Subjects had to wash hands after application unless treating lesions on the hands or fingernails.

Study drug was applied to all lesions, including newly appearing lesions and lesions that had improved during the study, if applicable.

Subjects were allowed, but not required, to treat fingernails, toenails, palms, soles, and scalp lesions with study drug; however, efficacy analyses did not include assessment of improvement of psoriasis in these areas. If using study drug on the scalp, no other treatment for scalp psoriasis was permitted during the study.

If there was residual cream visible on the disease affected lesional skin, then, the subject was instructed to continue to lightly rub the cream into the skin until it was no longer visible.

If study drug was applied to the subject by another person, that person was to thoroughly wash his/her hands after application. When possible, use of disposable gloves was recommended.

Subjects recorded the time of study drug application in their daily diary.

Subjects were to avoid swimming, bathing, showering, or strenuous activities for at least 2 hours after application of study drug.

On study visit days, study drug was applied in the clinic under the supervision of site personnel and after safety and efficacy assessments had been completed.

Note: The time of the dose and assessments on clinic visit days depended on the time of the clinic visit. Therefore, the timing of the clinic visit could have differed from the subject's chosen dosing time. The intention was to allow flexibility to accommodate subjects' schedules.

Subjects were instructed/reminded on how to apply study drug at each clinic visit (except during the final treatment visit).

Efficacy Assessments

To minimize interobserver variability, Investigators and evaluators/raters were trained on each of the required assessments during an Investigator meeting, site initiation visit, and/or by utilizing online assessments before enrolling subjects at their study site. Only trained evaluators/raters were permitted to perform the efficacy assessments. To the fullest extent possible, the same Investigator (or designated evaluator/rater) performed all efficacy assessments for an individual subject throughout the study. If it was not possible for the same evaluator/rater to continue performing assessments, it was recommended that the primary and subsequent evaluator/rater both examine and discuss their respective scoring during at least 1 visit.

Assessments Completed by the Investigator

Physician Global Assessment

The PGA was used to assess the primary efficacy endpoint in this study. The PGA is a clinical tool for assessing the current state/severity of a subject's psoriasis at a given time point. It is a static, 5 point, morphological assessment of overall disease severity, as determined by the Investigator, using the clinical characteristics of erythema, scaling, and plaque thickness/elevation as guidelines; higher PGA scores represent more severe disease (0=clear, 1=almost clear, 2=mild, 3=moderate, and 4=severe). The BSA affected was not considered in scoring of the PGA. Variations of the PGA are frequently used in clinical studies because it is a simple assessment, similar to those used in clinical practice. The PGA was performed first, prior to the % BSA, PASI, and LTS assessments.

Body Surface Area Affected

The % BSA affected is an estimate of the percentage of total involved skin with psoriasis. For the purpose of clinical estimation, the total palmar surface of the subject's palm and digits was assumed to be approximately equivalent to 1% BSA. The % BSA affected by psoriasis was evaluated (from 0% to 100%). Details on calculation of approximate % BSA involvement in each subject (total and individual) can be found in Appendix 2 of the protocol. Percent BSA is a static assessment made without reference to previous scores.

At Screening, Baseline, and for all efficacy assessments, lesions on the subject's scalp, palms, fingernails, toenails, and soles were not included in the calculation of % BSA affected as these areas were excluded from the efficacy analyses.

Psoriasis Area and Severity Index

The PASI scoring system is a widely used, standard, clinical tool for assessing the severity of psoriasis that takes into account the overall severity of erythema (redness), induration (plaque thickness), scale, and the extent of % BSA affected with psoriasis. The 3 clinical signs are each graded on a 5 point scale (0 to 4) and the % BSA affected is scored on a 7 point scale (0 to 6) for each of the 4 specified body regions (head, upper extremities, trunk, and lower extremities). Individual scores are multiplied by a weighted factor for each body region; the sum of these scores gives the overall PASI score. Higher scores indicate more severe disease. PASI is a static assessment made without reference to previous scores. Details on PASI calculation can be found in Appendix 2 of the protocol.

Assessments Completed by Subject

Dermatology Life Quality Index

Subjects completed the DLQI questionnaire. The DLQI is a simple, validated, dermatology specific, 10 question questionnaire to assess the impact of the disease on a subject's quality of life. The DLQI is an important outcome measure in dermatology clinical trials and is the most frequently used instrument in randomized, controlled dermatology trials. The DLQI can be analyzed as a total score (where a higher score indicates greater impairment in quality of life) and can also be scored for the following dimensions: Symptoms and Feelings (Items 1 and 2), Daily Activities (Items 3 and 4), Leisure (Items 5 and 6), Work and School (Item 7), Personal Relationships (Items 8 and 9), and Treatment (Item 10).

Patient Satisfaction Questionnaire

Subjects completed the Patient Satisfaction Questionnaire.

TABLE 1
Extension Study Efficacy Endpoints

| | |
|---|---|
| For subjects | Proportion of subjects who experienced PGA ≥ 2 at least |
| entering the | 1 time during the study |
| study with a | Proportion of subjects who never experienced PGA ≥ 2 |
| PGA score | throughout the study |
| of clear (0): | Time to first worsening (PGA ≥ 2), defined as days from |
| | the date of Visit 1 to the date of first occurrence of |
| | PGA ≥ 2. Subjects who never experienced PGA ≥ 2 |
| | throughout the study were censored at the date of their |
| | last PGA assessment. |
| For subjects | Proportion of subjects who achieved PGA = 0 at least 1 |
| entering the | time during the study |
| study with a | Proportion of subjects who never experienced PGA = 0 |
| PGA score ≥ | throughout the study |
| 1: | Time to achieving a PGA = 0, defined as days from date |
| | of Visit 1 to the date of first occurrence of PGA = 0. |
| | Subjects who never achieved PGA = 0 throughout the |
| | study were censored at the date of their last PGA |
| | assessment. |
| For subjects | Proportion of subjects who achieved PGA = 0 or 1 at |
| entering the | least 1 time during the study |
| study with a | Proportion of subjects who never achieved PGA = 0 or |
| PGA score ≥ | 1 throughout the study |
| 2: | |
| Endpoints | Duration (days) of treatment episodes, defined as time |
| summarized | (days) from date of each PGA ≥ 2 (or PGA ≥ 1 for the |
| over subjects | first episode) to 1 day before each subsequent treatment |
| in the ITT | success (PGA = 0). The date of the last PGA assessment |
| population: | was used as the end date for episodes ongoing at the end |
| | of this extension study. All 1st episodes were |
| | summarized, then all 2nd episodes, etc. Additionally, |
| | each subject was characterized by an average duration of |
| | episodes and these averages were summarized over the |
| | entire ITT population. |
| | Duration (days) of treatment successes, defined as time |
| | (days) from date of PGA = 0 to 1 day before each PGA ≥ |
| | 2. The date of the last PGA assessment was used as the |
| | end date for episodes of treatment success ongoing at the |
| | end of this extension study. All 1st treatment successes |
| | were summarized, then all 2nd treatment successes, etc. |
| | Additionally, each subject was characterized by an |
| | average duration of treatment success and these averages |
| | were summarized over the entire ITT population. Both |
| | durations of treatment episodes/successes were |
| | summarized by Baseline PGA scores. |
| | PGA scores and change from baseline values by visit |
| | (OC and LOCF) |
| | Absolute values, change and percent change from |
| | baseline in % BSA affected by visit (OC and LOCF) |
| | Absolute values, change and percent change from |
| | baseline in PASI score by visit (OC and LOCF) |
| | Proportion of subjects with ≥ 50% improvement in PASI |
| | score (PASI50) from Baseline by visit (OC and LOCF) |
| | Proportion of subjects with ≥ 75% improvement in PASI |
| | score (PASI75) from Baseline by visit (OC and LOCF) |
| | Proportion of subjects with ≥ 90% improvement in PASI |
| | score (PASI90) from Baseline by visit (OC and LOCF) |

All efficacy parameters were summarized over the ITT analysis set overall and by DB assigned treatment group. Summaries of efficacy endpoints by visit were performed using OC and LOCF methods.

Time to event parameters, such as the time from treatment success to subsequent worsening (PGA≥2) or duration of treatment episode, were summarized by the Kaplan Meier product limit method using OC.

The remaining efficacy endpoints were summarized descriptively: continuous data included the mean, SD, minimum, maximum, median, and number of observations; descriptive summary statistics for categorical data included frequency counts and percentages.

For the following efficacy outcomes:

Proportions of subjects who achieved PGA=0 at least 1 time during the study (for subjects entering the study with a PGA≥1)

Proportions of subjects who experienced PGA≥2 at least 1 time during the study (for subjects entering the study with a PGA=0)

PGA scores at Week 40 (LOCF)

% BSA affected absolute value and change from baseline to Week 40 (LOCF)

PASI absolute value and change from baseline to Week 40 (LOCF)

Subgroup analyses were generated for Baseline PGA, age (<65 and ≥65 years old), sex, race (White, Other), Baseline % BSA affected (<10%, ≥10%), duration of disease (<5 years, 5 to 10 years, >10 years), country (United States and Canada).

Health Related Quality of Life

The DLQI is a 10 item subject reported outcome, 6 domain measure that assessed the extent to which the skin condition had affected the subject's quality of life over the past week. The total score (0 to 30) is the sum of 10 questions with each ranging from 0 to 3. The DLQI was assessed at 4 week intervals. If one question was left unanswered, this was scored as 0 and the scores were summed and expressed as usual out of a maximum of 30. If ≥2 questions were left unanswered, the total DLQI score was considered missing.

When summarizing the domain scores, if a domain contains 2 questions and the answer to one of the questions was missing, that domain was scored as the answer to the non-missing question. If all the answers to questions in a domain were missing, the domain was not scored.

DLQI total score and sub scores on DLQI domains and their changes from baseline as well as percent change from baseline values were summarized descriptively. Additional details of the analysis of the DLQI are provided in the SAP.

Patient Satisfaction Questionnaire

Responses for each question of the Patient Satisfaction Questionnaire assessed at Week 40 (or Early Termination visit) were summarized overall and by DB assigned treatment group.

Disposition of Subjects is described in Table 2.

TABLE 2
Baseline Patient Demographics and Characteristics in Phase 3

| | Overall | Tapinarof→Tapinarof* | Vehicle→Tapinarof* |
|---|---|---|---|
| | (n = 763) | (n = 508) | (n = 255) |
| Age, years, | 50.7 (12.9) | 50.5 (12.9) | 51.0 (12.9) |
| mean (SD) | | | |
| Male, n (%) | 448 (58.7) | 304 (59.8) | 144 (56.5) |
| Weight, kg, | 92.4 (23.9) | 92.6 (25.1) | 92.1 (21.3) |
| mean (SD) | | | |
| BMI, kg/m², | 31.7 (7.7) | 31.6 (8.1) | 31.8 (7.0) |
| mean (SD) | | | |
| PGA, n (%)^{†} | | | |
| 0—Clear | 79 (10.4) | 74 (14.6) | 5 (2.0) |
| 1—Almost | 161 (21.1) | 144 (28.3) | 17 (6.7) |
| clear | | | |
| 2—Mild | 247 (32.4) | 187 (36.8) | 60 (23.5) |
| 3—Moderate | 249 (32.6) | 93 (18.3) | 156 (61.2) |
| 4—Severe | 23 (3.0) | 7 (1.4) | 16 (6.3) |
| PASI, mean | 4.8 (4.7) | 3.3 (3.5) | 7.7 (5.4) |
| (SD) | | | |
| BSA affected, | 4.6 (5.6) | 3.3 (4.7) | 7.3 (6.2) |
| %, mean | | | |
| (SD) | | | |

*Tapinarof Tapinarof: patients previously assigned to tapinarof in the pivotal trials who enrolled in Phase 3; Vehicle Tapinarof: patients previously assigned to vehicle in the pivotal trials who enrolled in Phase 3.
^{†}Four patients (3 tapinarof, 1 vehicle) did not have a baseline PGA, PASI, and BSA value and are listed as missing. ITT population.
BMI, body mass index;
BSA, body surface area;
ITT, intention-to-treat;
PASI, Psoriasis Area and Severity Index;
PGA, Physician Global Assessment,
SD, standard deviation.

A total of 763 subjects were enrolled from the DB, pivotal Phase 3 studies (Study DMVT 505 3001 and Study DMVT 5050 3002) of which 508 subjects received tapinarof cream, 1% and 255 subjects received vehicle cream. This is an interim analysis based on a cut of the data that occurred on 25 Nov. 2020. Data from all enrolled subjects at the time of the data cut are included in this interim analysis which includes 671 subjects who completed assessments up to 14 weeks and 349 subjects who completed assessments up to 40 weeks. The combination of subjects treated during one of the 2 DB, pivotal Phase 3 studies plus this extension study results in 235 and 450 subjects who were treated with the intended clinical dose and followed for AEs for a least 1 year or 6 months, respectively, as per ICH E1 guidelines. An additional 114 subjects who were previously randomized to vehicle cream in the DB, pivotal Phase 3 studies have also been exposed to tapinarof cream, 1% for at least 6 months in this extension study.

Although all subjects enrolled in this extension study received tapinarof cream, 1% if they met the PGA criteria, data are presented using the 2 randomized cohorts from the DB, pivotal Phase 3 studies: tapinarof cream, 1% and vehicle cream. Data from both cohorts combined are presented as overall.

A total of 763 subjects with chronic psoriasis were enrolled in this extension study, and as of the data cutoff date, 743 subjects received and 20 subjects had not yet received treatment with tapinarof cream, 1%. Seventy eight subjects entered this 40 week extension study with a PGA=0.

Of the 763 subjects, 260 (34.1%) completed the study (tapinarof cream, 1%: 34.3%; vehicle cream: 33.7%). Two hundred and thirteen subjects permanently discontinued this extension study. The most common reasons for premature treatment discontinuation prior to Week 40 were withdrawal of consent (10.9% overall; 9.6% and 13.3% in the tapinarof cream, 1% and vehicle cream, respectively), lost to follow up (6.2% overall; 6.7% and 5.1%, respectively), and AEs (5.8% overall; 5.7% and 5.9%, respectively).

Of the total enrolled ITT population, 763 subjects participated in this extension study for a mean (SD) of 231.6 (87.62) days; the number of days in the study was similar between the 2 DB assigned treatment groups.

Efficacy Analysis

All efficacy endpoints were analyzed using the ITT population.

Note: Visit 1 of this extension study is considered the Baseline visit which is also the last assessment completed as part of the DB, pivotal Phase 3 studies (Week 12). There was also a Baseline visit at the beginning of the DB, pivotal Phase 3 studies.

Upon entering this extension study, 4 subjects had missing Baseline PGA, % BSA, and PASI values. Subject 1034 004 signed an ICF prior to being deemed ineligible; this subject was discontinued prior to receiving tapinarof cream, 1%. Due to factors related to COVID 19 limiting in clinic visits, the Baseline visit for Subject 1038 016 was conducted virtually and was conducted by phone for Subject 1905 009 and Subject 2002 007.

Proportion of Subjects who Experienced a PGA≥2 at Least 1 Time in the Study and the Median Time from Visit 1 Date to First Worsening for Subjects Entering this Extension Study with a PGA=0

Table 3 presents the proportion of subjects who experienced a PGA≥2 at least once after entering this extension study with a PGA=0; Table 4 presents the time to that PGA≥2. Of the 78 subjects who entered this extension study with a PGA=0, 73 had been randomized to tapinarof cream, 1% in the DB, pivotal Phase 3 studies and 5 had been randomized to vehicle cream. A PGA≥2 was experienced by 75.6% of these subjects (n=78) at least once at the data cutoff date for this interim analysis. Seventeen subjects randomized to tapinarof cream, 1% in the DB, pivotal Phase 3 studies did not experience a PGA≥2 at the time of the interim analysis. Of this group, 2 subjects completed the study, 10 were ongoing, and 5 had an early termination. In addition, of the 5 subjects previously randomized to vehicle cream who entered with PGA=0, there were 2 subjects who did not receive tapinarof cream, 1% at the time of the data cutoff date.

The median (95% confidence interval [CI]) time to first worsening (PGA≥2) was 115 days (85.0, 162.0; Table 3).

TABLE 3
Analysis of Efficacy Endpoint, Proportion of Subjects Experiencing
PGA ≥ 2; Subjects Entering the Extension Study with PGA = 0 (ITT
Population, Observed Cases)

| | Tapinarof | Vehicle | |
|---|---|---|---|
| | Cream, 1% | Cream | |
| | (Pivotal) | (Pivotal) | Overall |
| | (N = 73) | (N = 5) | (N = 78) |
| Proportion of subjects who | 56/73 (76.7) | 3/5 (60.0) | 59/78 (75.6) |
| experienced PGA ≥ 2 at least 1 | | | |
| time during the study, n/N (%) | | | |
| Proportion of subjects who never | 17/73 (23.3) | 2/5 (40.0) | 19/78 (24.4) |
| experienced PGA ≥ 2 throughout | | | |
| the study, n/N (%) | | | |

DB = double blinded;
ITT = Intent-to-Treat;
PGA = Physician Global Assessment.

TABLE 4
Analysis of Efficacy Endpoint, Time to First Worsening (PGA ≥ 2);
Subjects Entering this Extension Study with a PGA = 0 (ITT Population,
Observed Cases)

| | | |
|---|---|---|
| Tapinarof | Vehicle | |
| Cream, 1% | Cream | |
| (Pivotal) | (Pivotal) | Overall |
| (N = 73) | (N = 5) | (N = 78) |

Time to First Worsening (PGA ≥ 2; Days) [1]
Kaplan-Meier Estimates

| | | | |
|---|---|---|---|
| N | 73 | 5 | 78 |
| Number of | 56 | 3 | 59 |
| Subjects with | | | |
| 1st Worsening | | | |
| 25th percentile | 54 (29.0, 76.0) | 66 (42.0, NE) | 54 (29.0, 66.0) |
| (95% CI) | | | |
| Median (95% | 114 (85.0, 142.0) | 281 (42.0, NE) | 115 (85.0, 162.0) |
| CI) | | | |
| 75th percentile | 196 (168.0, 309.0) | NE (NE, NE) | 222 (169.0, 309.0) |
| (95% CI) | | | |

[1] Days from date of Baseline visit to the date of first occurrence of PGA ≥ 2. Subjects who never experienced PGA ≥ 2 throughout the study were censored at the date of their last PGA assessment.
CI = confidence interval;
DB = double blinded;
ITT = Intent-to-Treat;
NE = not estimable;
PGA = Physician Global Assessment.

Proportion of Subjects who Achieved a PGA=0 at Least 1 Time in the Study and the Median Time from Baseline Visit to First Achieving a PGA=0 for Subjects Entering this Extension Study with a PGA≥1

Table 5 presents the proportion of subjects who achieved a PGA=0 at least once after entering this extension study with a PGA≥1; Table 6 presents the time to that PGA=0. Of the 681 subjects who entered this extension study with a PGA≥1, 32.5% of those subjects experienced a PGA=0 at least once during the study. The proportions were similar between the DB assigned treatment groups (tapinarof cream, 1%: 32.2%; vehicle cream: 32.9%).

The median (95% CI) time to first achieving PGA=0 was not estimable; however, the 25th percentile (95% CI) was 141 days (113.0, 169.0). Subjects who were randomized to vehicle cream during the DB, pivotal Phase 3 studies achieved their first PGA=0 (169 days) later than those subjects who were randomized to tapinarof cream, 1% (113 days; Table 6).

TABLE 5
Analysis of Efficacy Endpoint, Proportion of Subjects Achieving
PGA = 0; Subjects Entering the Extension Study with a PGA ≥ 1
(ITT Population, Observed Cases).

| | Tapinarof | Vehicle | |
|---|---|---|---|
| | Cream, 1% | Cream | |
| | (Pivotal) | (Pivotal) | Overall |
| | (N = 432) | (N = 249) | (N = 681) |
| Proportion of subjects | 139/432 (32.2) | 82/249 (32.9) | 221/681 (32.5) |
| who achieved PGA = 0 | | | |
| at least 1 time during the | | | |
| study, n/N (%) | | | |
| Proportion of subjects | 293/432 (67.8) | 167/249 (67.1) | 460/681 (67.5) |
| who never achieved | | | |
| PGA = 0 throughout the | | | |
| study, n/N (%) | | | |

DB = double blinded;
ITT = Intent-to-Treat;
PGA = Physician Global Assessment

TABLE 6
Analysis of Efficacy Endpoint, Time to First Achieving PGA = 0;
Subjects Entering the Extension Study with PGA ≥ 1 (ITT Population,
Observed Cases)

| | | |
|---|---|---|
| Tapinarof | Vehicle | |
| Cream, 1% | Cream | |
| (Pivotal) | (Pivotal) | Overall |
| (N = 432) | (N = 249) | (N = 681) |

Time to First Achieving PGA = 0 (Days) [1]
Kaplan-Meier Estimates

| | | | |
|---|---|---|---|
| N | 432 | 249 | 681 |
| Number of Subjects with | 139 | 82 | 221 |
| 1st Achieving | | | |
| 25th percentile (95% CI) | 113 (89.0, | 169 (119.0, | 141 (113.0, |
| | 148.0) | 206.0) | 169.0) |
| Median (95% CI) | NE (NE, NE) | 313 (283.0, | NE (NE, NE) |
| | | NE) | |
| 75th percentile (95% CI) | NE (NE, NE) | NE (NE, NE) | NE (NE, NE) |

CI = confidence interval;
DB = double blinded;
ITT = Intent-to-Treat;
NE = not estimable;
PGA = Physician Global Assessment.

Proportion of Subjects who Achieved PGA=0 or 1 at Least 1 Time in the Study for Subjects Entering the Extension Study with PGA≥2

Table 7 presents the proportion of subjects achieving a PGA=0 or 1 who entered this extension study with PGA≥2. Of the 520 subjects who entered this extension study with a PGA≥2, 57.3% of those subjects experienced a PGA=0 or 1 at least once during the study. The proportions were similar between the DB assigned treatment groups (tapinarof cream, 1%: 53.5%; vehicle cream: 62.1%).

TABLE 7
Analysis of Efficacy Endpoint, Proportion of Subjects Achieving a
PGA = 0 or 1; Subjects Entering the Extension Study with PGA ≥ 2
(ITT Population, Observed Cases)

| | Tapinarof | Vehicle | |
|---|---|---|---|
| | Cream, 1% | Cream | |
| | (Pivotal) | (Pivotal) | Overall |
| | (N = 288) | (N = 232) | (N = 520) |
| Proportion of subjects | 154/288 (53.5) | 144/232 (62.1) | 298/520 (57.3) |
| who achieved PGA = | | | |
| 0 or 1 at least 1 time | | | |
| during the study, n/N | | | |
| (%) | | | |
| Proportion of subjects | 134/288 (46.5) | 88/232 (37.9) | 222/520 (42.7) |
| who never achieved | | | |
| PGA = 0 or 1 | | | |
| throughout the study, | | | |
| n/N (%) | | | |

DB = double blinded;
ITT = Intent-to-Treat;
PGA = Physician Global Assessment.

Duration of Treatment Episodes

The duration of each treatment episode is defined as the time from each PGA≥2 (or PGA≥1 for the first episode [Baseline]) to each subsequent treatment success (PGA=0). Table 8 presents the durations of treatment episodes. Treatment episodes that were ongoing as of the last known PGA evaluation were right censored, thus the treatment duration was stopped due to the subject reaching the end of the study, not because the subject achieved a PGA=0. At the time of the data cutoff date for this interim analysis, 96.3% of subjects required one treatment episode with a mean (SD) duration of 163.1 (97.10) days. The treatment episode data were right censored for 66.4% of these subjects. The mean (SD) duration of the first treatment episode was approximately 19 days longer in the group of subjects who were randomized to vehicle cream in the DB, pivotal Phase 3 studies (175.5 [92.74] days) compared with subjects randomized to tapinarof cream, 1% (156.6 [98.77] days).

The overall mean (SD) durations of the second and third treatment episodes continued to shorten (74.0 [55.35] and 50.4 [41.38] days, respectively); however, this is primarily due to the number of subjects' data requiring right censoring for this interim analysis (60.2% and 75.0% for the second and third treatment episodes, respectively). Only 1 subject required a fourth treatment episode.

Overall, the mean (SD) total duration of treatment episodes for each subject was 177.6 (90.38) days.

TABLE 8
Analysis of Efficacy Endpoint, Duration of Treatment Episodes (ITT
Population, Observed Cases)

| | | |
|---|---|---|
| Tapinarof | Vehicle | |
| Cream, 1% | Cream | |
| (Pivotal) | (Pivotal) | Overall |
| (N = 508) | (N = 255) | (N = 763) |

Duration of 1st Episode (days)

| | | | |
|---|---|---|---|
| Number of subjects | 484 | 251 | 735 |
| Number of subjects without | 166 | 81 | 247 |
| censoring [1] | | | |
| Number of subjects with right | 318 | 170 | 488 |
| censoring [1] | | | |
| Mean (SD) | 156.6 (98.77) | 175.5 (92.74) | 163.1 (97.10) |
| Median | 147.5 | 196.0 | 169.0 |
| Minimum, Maximum | 1, 307 | 1, 294 | 1, 307 |

Duration of 2nd Episode (days)

| | | | |
|---|---|---|---|
| Number of subjects | 94 | 39 | 133 |
| Number of subjects without | 34 | 19 | 53 |
| censoring [1] | | | |
| Number of subjects with right | 60 | 20 | 80 |
| censoring [1] | | | |
| Mean (SD) | 80.7 (58.54) | 57.8 (43.31) | 74.0 (55.35) |
| Median | 60.5 | 38.0 | 57.0 |
| Minimum, Maximum | 1, 225 | 1, 169 | 1, 225 |

Duration of 3rd Episode (days)

| | | | |
|---|---|---|---|
| Number of subjects | 9 | 7 | 16 |
| Number of subjects without | 2 | 2 | 4 |
| censoring [1] | | | |
| Number of subjects with right | 7 | 5 | 12 |
| censoring [1] | | | |
| Mean (SD) | 49.6 (52.51) | 51.6 (24.51) | 50.4 (41.38) |
| Median | 28.0 | 56.0 | 32.5 |
| Minimum, Maximum | 1, 138 | 26, 86 | 1, 138 |

Duration of 4th Episode (days)

| | | | |
|---|---|---|---|
| Number of subjects | 0 | 1 | 1 |
| Number of subjects with right | 0 | 1 | 1 |
| censoring [1] | | | |
| Mean (SD) | — | 1.0 (NE) | 1.0 (NE) |
| Median | — | 1.0 | 1.0 |
| Minimum, Maximum | — | 1, 1 | 1, 1 |

Duration of Last Episode for Each Subject (days)

| | | | |
|---|---|---|---|
| Number of subjects | 484 | 251 | 735 |
| Number of subjects without | 99 | 55 | 154 |
| censoring [1] | | | |
| Number of subjects with right | 385 | 196 | 581 |
| censoring [1] | | | |
| Mean (SD) | 157.6 (99.25) | 171.4 (96.61) | 162.4 (98.51) |
| Median | 168.0 | 195.0 | 169.0 |
| Minimum, Maximum | 1, 307 | 1, 294 | 1, 307 |

Average Duration of Episodes for Each Subject (days)

| | | | |
|---|---|---|---|
| N | 484 | 251 | 735 |
| Mean (SD) | 157.1 (97.16) | 173.4 (93.60) | 162.7 (96.20) |
| Median | 140.5 | 195.0 | 168.0 |
| Minimum, Maximum | 1, 307 | 1, 294 | 1, 307 |

Total Duration of Treatment Episodes for Each Subject (days)

| | | | |
|---|---|---|---|
| N | 484 | 251 | 735 |
| Mean (SD) | 173.2 (92.39) | 185.9 (85.95) | 177.6 (90.38) |
| Median | 196.0 | 197.0 | 197.0 |
| Minimum, Maximum | 1, 307 | 1, 294 | 1, 307 |

[1] Right censoring indicates that the treatment episode was ongoing as of the last known PGA evaluation that treatments could be scheduled.
DB = double blinded;
ITT = Intent-to-Treat;
NE = not estimable;
PGA = Physician Global Assessment;
SD = standard deviation.

Table 9 presents the durations of treatment episodes by Baseline visit PGA score upon entering this extension study. Overall, the mean (SD) duration of the first treatment episode increased with increasing Baseline PGA score: PGA=0: 92.9 (67.74) days, PGA=1: 133.5 (100.06) days, PGA=2: 177.8 (92.43) days, PGA=3: 180.8 (93.84) days, and PGA=4: 187.4 (102.04) days. Of note, the percentage of subjects affected by right censoring also increased with increasing PGA score (PGA=0 [40.0%], PGA=1 [47.8%], PGA=2 [72.1%], PGA=3 [77.1%], and PGA=4 [82.6%]). Overall, the mean (SD) total duration of treatment for each subject were PGA=0: 109.2 (61.94) days, PGA=1: 165.9 (90.32) days, PGA=2: 188.8 (87.61) days, PGA=3: 187.9 (90.62) days, and PGA=4: 191.1 (99.63) days.

TABLE 9
Analysis of Efficacy Endpoint, Duration of Treatment Episodes by
Baseline PGA Score (ITT Population, Observed Cases)
Baseline PGA = 0

| | | |
|---|---|---|
| Tapinarof | Vehicle | |
| Cream, 1% | Cream | |
| (Pivotal) | (Pivotal) | Overall |
| (N = 73) | (N = 5) | (N = 78) |

Duration of 1st Episode (days)

| | | | |
|---|---|---|---|
| Number of subjects | 53 | 2 | 55 |
| Number of subjects | 31 | 2 | 33 |
| without censoring [1] | | | |
| Number of subjects with | 22 | 0 | 22 |
| right censoring [1] | | | |
| Mean (SD) | 91.7 (66.66) | 126.0 (120.21) | 92.9 (67.74) |
| Median | 65.0 | 126.0 | 65.0 |
| Minimum, Maximum | 25, 253 | 41, 211 | 25, 253 |

Duration of 2nd Episode (days)

| | | | |
|---|---|---|---|
| Number of subjects | 18 | 1 | 19 |
| Number of subjects | 10 | 1 | 11 |
| without censoring [1] | | | |
| Number of subjects with | 8 | 0 | 8 |
| right censoring [1] | | | |
| Mean (SD) | 44.9 (26.43) | 28.0 (NE) | 44.1 (25.97) |
| Median | 43.0 | 28.0 | 42.0 |
| Minimum, Maximum | 1, 92 | 28, 28 | 1, 92 |

Duration of 3rd Episode (days)

| | | | |
|---|---|---|---|
| Number of subjects | 1 | 0 | 1 |
| Number of subjects with | 1 | 0 | 1 |
| right censoring [1] | | | |
| Mean (SD) | 61.0 (NE) | — | 61.0 (NE) |
| Median | 61.0 | — | 61.0 |
| Minimum, Maximum | 61, 61 | — | 61, 61 |

Duration of Last Episode for Each Subject (days)

| | | | |
|---|---|---|---|
| Number of subjects | 53 | 2 | 55 |
| Number of subjects | 22 | 2 | 24 |
| without censoring [1] | | | |
| Number of subjects with | 31 | 0 | 31 |
| right censoring [1] | | | |
| Mean (SD) | 88.8 (67.68) | 119.5 (129.40) | 89.9 (68.95) |
| Median | 63.0 | 119.5 | 63.0 |
| Minimum, Maximum | 1, 253 | 28, 211 | 1, 253 |

Average Duration of Episodes for Each Subject (days)

| | | | |
|---|---|---|---|
| N | 53 | 2 | 55 |
| Mean (SD) | 90.2 (65.67) | 122.8 (124.80) | 91.4 (66.93) |
| Median | 63.0 | 122.8 | 63.0 |
| Minimum, Maximum | 27, 253 | 35, 211 | 27, 253 |

Total Duration of Treatment Episodes for Each Subject (days)

| | | | |
|---|---|---|---|
| N | 53 | 2 | 55 |
| Mean (SD) | 108.1 (61.26) | 140.0 (100.41) | 109.2 (61.94) |
| Median | 94.0 | 140.0 | 94.0 |
| Minimum, Maximum | 27, 253 | 69, 211 | 27, 253 |

Baseline PGA = 1

| | | |
|---|---|---|
| Tapinarof | Vehicle | |
| Cream, 1% | Cream | |
| (Pivotal) | (Pivotal) | Overall |
| (N = 144) | (N= 17) | (N = 161) |

Duration of 1st Episode (days)

| | | | |
|---|---|---|---|
| Number of subjects | 144 | 17 | 161 |
| Number of subjects | 75 | 9 | 84 |
| without censoring [1] | | | |
| Number of subjects with | 69 | 8 | 77 |
| right censoring [1] | | | |
| Mean (SD) | 132.6 (99.69) | 141.1 (105.95) | 133.5 (100.06) |
| Median | 85.5 | 112.0 | 87.0 |
| Minimum, Maximum | 1, 291 | 27, 284 | 1, 291 |

Duration of 2nd Episode (days)

| | | | |
|---|---|---|---|
| Number of subjects | 45 | 4 | 49 |
| Number of subjects | 18 | 1 | 19 |
| without censoring [1] | | | |
| Number of subjects with | 27 | 3 | 30 |
| right censoring [1] | | | |
| Mean (SD) | 97.9 (62.19) | 104.0 (61.27) | 98.4 (61.51) |
| Median | 83.0 | 109.5 | 85.0 |
| Minimum, Maximum | 1, 225 | 28, 169 | 1, 225 |

Duration of 3rd Episode (days)

| | | | |
|---|---|---|---|
| Number of subjects | 7 | 0 | 7 |
| Number of subjects | 2 | 0 | 2 |
| without censoring [1] | | | |
| Number of subjects with | 5 | 0 | 5 |
| right censoring [1] | | | |
| Mean (SD) | 54.9 (56.83) | — | 54.9 (56.83) |
| Median | 28.0 | — | 28.0 |
| Minimum, Maximum | 1, 138 | — | 1, 138 |

Duration of Last Episode for Each Subject (days)

| | | | |
|---|---|---|---|
| Number of subjects | 144 | 17 | 161 |
| Number of subjects | 43 | 6 | 49 |
| without censoring [1] | | | |
| Number of subjects with | 101 | 11 | 112 |
| right censoring [1] | | | |
| Mean (SD) | 145.2 (96.38) | 159.0 (93.38) | 146.7 (95.87) |
| Median | 144.0 | 134.0 | 141.0 |
| Minimum, Maximum | 1, 291 | 28, 284 | 1, 291 |

Average Duration of Episodes for Each Subject (days)

| | | | |
|---|---|---|---|
| Number of subjects | 144 | 17 | 161 |
| Mean (SD) | 138.9 (94.99) | 150.0 (97.54) | 140.1 (95.01) |
| Median | 112.0 | 112.0 | 112.0 |
| Minimum, Maximum | 1, 291 | 28, 284 | 1, 291 |

Total Duration of Treatment Episodes for Each Subject (days)

| | | | |
|---|---|---|---|
| Number of subjects | 144 | 17 | 161 |
| Mean (SD) | 165.9 (90.68) | 165.5 (89.94) | 165.9 (90.32) |
| Median | 194.5 | 140.0 | 193.0 |
| Minimum, Maximum | 1, 291 | 45, 284 | 1, 291 |

Baseline PGA = 2 (Mild)

| | | |
|---|---|---|
| Tapinarof | Vehicle | |
| Cream, 1% | Cream | |
| (Pivotal) | (Pivotal) | Overall |
| (N = 188) | (N = 60) | (N = 248) |

Duration of 1st Episode (days)

| | | | |
|---|---|---|---|
| Number of subjects | 187 | 60 | 247 |
| Number of subjects | 47 | 22 | 69 |
| without censoring [1] | | | |
| Number of subjects with | 140 | 38 | 178 |
| right censoring [1] | | | |
| Mean (SD) | 179.2 (94.49) | 173.2 (86.28) | 177.8 (92.43) |
| Median | 197.0 | 194.0 | 197.0 |
| Minimum, Maximum | 1, 307 | 27, 294 | 1, 307 |

Duration of 2nd Episode (days)

| | | | |
|---|---|---|---|
| Number of subjects | 26 | 13 | 39 |
| Number of subjects | 5 | 7 | 12 |
| without censoring [1] | | | |
| Number of subjects with | 21 | 6 | 27 |
| right censoring [1] | | | |
| Mean (SD) | 78.2 (54.89) | 46.0 (42.39) | 67.5 (52.79) |
| Median | 84.5 | 27.0 | 59.0 |
| Minimum, Maximum | 1, 180 | 1, 120 | 1, 180 |

Duration of 3rd Episode (days)

| | | | |
|---|---|---|---|
| Number of subjects | 1 | 2 | 3 |
| Number of subjects | 0 | 1 | 1 |
| without censoring [1] | | | |
| Number of subjects with | 1 | 1 | 2 |
| right censoring [1] | | | |
| Mean (SD) | 1.0 (NE) | 42.5 (19.09) | 28.7 (27.50) |
| Median | 1.0 | 42.5 | 29.0 |
| Minimum, Maximum | 1, 1 | 29, 56 | 1, 56 |

Duration of Last Episode for Each Subject (days)

| | | | |
|---|---|---|---|
| Number of subjects | 187 | 60 | 247 |
| Number of subjects | 25 | 15 | 40 |
| without censoring [1] | | | |
| Number of subjects with | 162 | 45 | 207 |
| right censoring [1] | | | |
| Mean (SD) | 175.4 (98.91) | 166.2 (95.09) | 173.2 (97.88) |
| Median | 197.0 | 194.0 | 197.0 |
| Minimum, Maximum | 1, 307 | 1, 294 | 1, 307 |

Average Duration of Episodes for Each Subject (days)

| | | | |
|---|---|---|---|
| Number of subjects | 187 | 60 | 247 |
| Mean (SD) | 177.3 (95.45) | 169.8 (89.68) | 175.5 (93.95) |
| Median | 197.0 | 194.0 | 197.0 |
| Minimum, Maximum | 1, 307 | 24, 294 | 1, 307 |

Total Duration of Treatment Episodes for Each Subject (days)

| | | | |
|---|---|---|---|
| Number of Subjects | 187 | 60 | 247 |
| Mean (SD) | 190.1 (90.58) | 184.6 (78.19) | 188.8 (87.61) |
| Median | 223.0 | 196.5 | 200.0 |
| Minimum, Maximum | 1, 307 | 27, 294 | 1, 307 |

Baseline PGA = 3 (Moderate)

| | | |
|---|---|---|
| Tapinarof | Vehicle | |
| Cream, 1% | Cream | |
| (Pivotal) | (Pivotal) | Overall |
| (N = 93) | (N = 156) | (N = 249) |

Duration of 1st Episode (days)

| | | | |
|---|---|---|---|
| Number of subjects | 93 | 156 | 249 |
| Number of subjects | 11 | 46 | 57 |
| without censoring [1] | | | |
| Number of subjects with | 82 | 110 | 192 |
| right censoring [1] | | | |
| Mean (SD) | 187.1 (95.90) | 177.1 (92.70) | 180.8 (93.84) |
| Median | 198.0 | 195.5 | 197.0 |
| Minimum, Maximum | 1, 305 | 1, 292 | 1, 305 |

Duration of 2nd Episode (days)

| | | | |
|---|---|---|---|
| Number of subjects | 5 | 20 | 25 |
| Number of subjects | 1 | 10 | 11 |
| without censoring [1] | | | |
| Number of subjects with | 4 | 10 | 14 |
| right censoring [1] | | | |
| Mean (SD) | 68.2 (77.66) | 56.4 (37.29) | 58.8 (46.15) |
| Median | 30.0 | 55.5 | 55.0 |
| Minimum, Maximum | 1, 196 | 1, 142 | 1, 196 |

Duration of 3rd Episode (days)

| | | | |
|---|---|---|---|
| Number of subjects | 0 | 5 | 5 |
| Number of subjects | 0 | 1 | 1 |
| without censoring [1] | | | |
| Number of subjects with | 0 | 4 | 4 |
| right censoring [1] | | | |
| Mean (SD) | — | 55.2 (27.44) | 55.2 (27.44) |
| Median | — | 57.0 | 57.0 |
| Minimum, Maximum | — | 26, 86 | 26, 86 |

Duration of 4th Episode (days)

| | | | |
|---|---|---|---|
| Number of subjects | 0 | 1 | 1 |
| Number of subjects with | 0 | 1 | 1 |
| right censoring [1] | | | |
| Mean (SD) | — | 1.0 (NE) | 1.0 (NE) |
| Median | — | 1.0 | 1.0 |
| Minimum, Maximum | — | 1, 1 | 1, 1 |

Duration of Last Episode for Each Subject (days)

| | | | |
|---|---|---|---|
| Number of subjects | 93 | 156 | 249 |
| Number of subjects | 7 | 31 | 38 |
| without censoring [1] | | | |
| Number of subjects with | 86 | 125 | 211 |
| right censoring [1] | | | |
| Mean (SD) | 182.2 (99.93) | 171.4 (97.39) | 175.4 (98.29) |
| Median | 198.0 | 195.0 | 197.0 |
| Minimum, Maximum | 1, 305 | 1, 292 | 1, 305 |

Average Duration of Episodes for Each Subject (days)

| | | | |
|---|---|---|---|
| Number of subjects | 93 | 156 | 249 |
| Mean (SD) | 184.7 (96.36) | 174.0 (94.06) | 178.0 (94.87) |
| Median | 198.0 | 195.0 | 197.0 |
| Minimum, Maximum | 1, 305 | 1, 292 | 1, 305 |

Total Duration of Treatment Episodes for Each Subject (days)

| | | | |
|---|---|---|---|
| Number of subjects | 93 | 156 | 249 |
| Mean (SD) | 190.8 (95.50) | 186.1 (87.86) | 187.9 (90.62) |
| Median | 200.0 | 197.5 | 198.0 |
| Minimum, Maximum | 1, 305 | 1, 292 | 1, 305 |

Baseline PGA = 4 (Severe)

| | | |
|---|---|---|
| Tapinarof | Vehicle | |
| Cream, 1% | Cream | |
| (Pivotal) | (Pivotal) | Overall |
| (N = 7) | (N= 16) | (N = 23) |

Duration of 1st Episode (days)

| Number of subjects | 7 | 16 | 23 |
|---|---|---|---|
| Number of subjects | | | |
| without censoring [1] | | | |
| Number of subjects with | 5 | 14 | 19 |
| right censoring [1] | | | |
| Mean (SD) | 132.9 (100.39) | 211.3 (96.13) | 187.4 (102.04) |
| Median | 113.0 | 267.0 | 225.0 |
| Minimum, Maximum | 16, 280 | 29, 287 | 16, 287 |

Duration of 2nd Episode (days)

| | | | |
|---|---|---|---|
| Number of subjects | 0 | 1 | 1 |
| Number of subjects with | 0 | 1 | 1 |
| right censoring [1] | | | |
| Mean (SD) | — | 85.0 (NE) | 85.0 (NE) |
| Median | — | 85.0 | 85.0 |
| Minimum, Maximum | — | 85, 85 | 85, 85 |

Duration of Last Episode for Each Subject (days)

| | | | |
|---|---|---|---|
| Number of subjects | 7 | 16 | 23 |
| Number of subjects | 2 | 1 | 3 |
| without censoring [1] | | | |
| Number of subjects with | 5 | 15 | 20 |
| right censoring [1] | | | |
| Mean (SD) | 132.9 (100.39) | 211.4 (96.04) | 187.5 (101.99) |
| Median | 113.0 | 267.0 | 225.0 |
| Minimum, Maximum | 16, 280 | 29, 287 | 16, 287 |

Average Duration of Episodes for Each Subject (days)

| | | | |
|---|---|---|---|
| Number of subjects | 7 | 16 | 23 |
| Mean (SD) | 132.9 (100.39) | 211.3 (96.08) | 187.5 (102.01) |
| Median | 113.0 | 267.0 | 225.0 |
| Minimum, Maximum | 16, 280 | 29, 287 | 16, 287 |

Total Duration of Treatment Episodes for Each Subject (days)

| | | | |
|---|---|---|---|
| Number of subjects | 7 | 16 | 23 |
| Mean (SD) | 132.9(100.39) | 216.6 (90.82) | 191.1 (99.63) |
| Median | 113.0 | 267.0 | 225.0 |
| Minimum, Maximum | 16, 280 | 29, 287 | 16, 287 |

[1] Right censoring indicates that the treatment episode was ongoing as of the last known PGA evaluation that treatments could have been scheduled.
DB = double blinded;
ITT = Intent-to-Treat;
NE = not estimable;
PGA = Physician Global Assessment;
SD = standard deviation

Duration of Treatment Success

The duration of treatment success (treatment-free interval or remittive response) is defined as the time from each PGA=0 to each subsequent worsening (PGA≥2). Table 10 presents the durations of treatment successes. Treatment successes that were ongoing as of the last known PGA evaluation were right censored resulting in the treatment-free interval being stopped due to the subject reaching the end of the study, not because the subject had worsening disease. As of the data cutoff date for this interim analysis, 299 subjects (39.2%) experienced at least one treatment success with a mean (SD) duration of 100.0 (78.49) days. The treatment success data were right censored for 37.8% of these subjects. The mean duration of the first treatment success was approximately 10 days longer in the group of subjects who were randomized to tapinarof cream, 1% in the DB, pivotal Phase 3 studies (103.0 [80.13] days) compared with subjects randomized to vehicle cream (92.8 [74.32] days.

The overall mean (SD) durations of the second and third treatment successes continued to shorten (65.4 [60.68] days and 30.0 [26.46] days, respectively); however, this is primarily due to the number of subjects' data requiring right censoring for this interim analysis (45.7% and 66.7% for the second and third treatment successes, respectively).

Overall, the mean (SD) total duration of treatment success for each subject was 119.3 (81.80) days.

TABLE 10
Analysis of Efficacy Endpoint, Duration of Treatment Success (ITT
Population, Observed Cases)

| | | |
|---|---|---|
| Tapinarof | Vehicle | |
| Cream, 1% | Cream | |
| (Pivotal) | (Pivotal) | Overall |
| (N = 508) | (N = 255) | (N = 763) |

Duration of 1st Success (days)

| | | | |
|---|---|---|---|
| Number of subjects | 212 | 87 | 299 |
| Number of subjects without | 140 | 46 | 186 |
| censoring [1] | | | |
| Number of subjects with | 72 | 41 | 113 |
| right censoring [1] | | | |
| Mean (SD) | 103.0 (80.13) | 92.8 (74.32) | 100.0 (78.49) |
| Median | 84.0 | 85.0 | 84.0 |
| Minimum, Maximum | 1, 309 | 1, 309 | 1, 309 |

Duration of 2nd Success (days)

| | | | |
|---|---|---|---|
| Number of subjects | 57 | 24 | 81 |
| Number of subjects without | 34 | 10 | 44 |
| censoring [1] | | | |
| Number of subjects with | 23 | 14 | 37 |
| right censoring [1] | | | |
| Mean (SD) | 76.5 (63.07) | 39.1 (45.75) | 65.4 (60.68) |
| Median | 56.0 | 28.0 | 41.0 |
| Minimum, Maximum | 1, 257 | 1, 170 | 1, 257 |

Duration of 3rd Success (days)

| | | | |
|---|---|---|---|
| Number of subjects | 12 | 3 | 15 |
| Number of subjects without | 4 | 1 | 5 |
| censoring [1] | | | |
| Number of subjects with | 8 | 2 | 10 |
| right censoring [1] | | | |
| Mean (SD) | 32.7 (28.40) | 19.3 (15.89) | 30.0 (26.46) |
| Median | 28.5 | 28.0 | 28.0 |
| Minimum, Maximum | 1, 85 | 1, 29 | 1, 85 |

Duration of Last Success for Each Subject (days)

| | | | |
|---|---|---|---|
| Number of subjects | 212 | 87 | 299 |
| Number of subjects without | 109 | 30 | 139 |
| censoring [1] | | | |
| Number of subjects with | 103 | 57 | 160 |
| right censoring [1] | | | |
| Mean (SD) | 101.5 (83.51) | 80.5 (77.32) | 95.4 (82.18) |
| Median | 82.5 | 56.0 | 64.0 |
| Minimum, Maximum | 1, 309 | 1, 309 | 1, 309 |

Average Duration of Treatment Success for Each Subject (days)

| | | | |
|---|---|---|---|
| Number of subjects | 212 | 87 | 299 |
| Mean (SD) | 102.4 (77.90) | 86.8 (72.58) | 97.9 (76.60) |
| Median | 84.0 | 64.0 | 84.0 |
| Minimum, Maximum | 1, 309 | 1, 309 | 1, 309 |

Total Duration of Treatment Success for Each Subject (days)

| | | | |
|---|---|---|---|
| Number of subjects | 212 | 87 | 299 |
| Mean (SD) | 125.4 (83.22) | 104.3 (76.62) | 119.3 (81.80) |
| Median | 116.5 | 97.0 | 112.0 |
| Minimum, Maximum | 1, 309 | 1, 309 | 1, 309 |

[1] Right censoring indicates that treatment success was ongoing as of the last known PGA evaluation.
DB = double blinded;
ITT = Intent-to-Treat;
PGA = Physician Global Assessment;
SD = standard deviation.

Table 11 presents the duration of treatment successes by Baseline visit PGA upon entering this extension study. Overall, the mean (SD) duration of the first treatment success decreased with increasing Baseline PGA score from PGA=0 to PGA=3: PGA=0: 125.1 (91.87) days, PGA=1: 107.2 (76.62) days, PGA=2: 90.4 (72.34) days, and PGA=3: 68.8 (57.40) days. The overall mean (SD) duration of the first treatment success was 104.5 (56.36) days for subjects with a Baseline PGA=4. Of note, the percentage of subjects affected by right censoring increased with increasing PGA score (PGA=0 [24.4%], PGA=1 [36.5%], PGA=2 [40.8%], PGA=3 [50.8%], and PGA=4 [75.0%]). Overall, the mean (SD) total duration of treatment success for each subject also decreased with increasing Baseline PGA score from PGA=0 to PGA=3: PGA=0: 167.9 (84.48) days, PGA=1: 124.5 (78.53) days, PGA=2: 98.0 (74.01) days, and PGA=3: 75.5 (57.18) days. The overall mean (SD) total duration of treatment success was 104.5 (56.36) days for subjects with a Baseline PGA=4.

TABLE 11
Analysis of Efficacy Endpoint, Duration of Treatment Success by Baseline
PGA Score (ITT Population, Observed Cases)
Baseline PGA = 0 (Clear)

| | | |
|---|---|---|
| Tapinarof | Vehicle | |
| Cream, 1% | Cream | |
| (Pivotal) | (Pivotal) | Overall |
| (N = 73) | (N = 5) | (N = 78) |

Duration of 1st Success (days)

| | | | |
|---|---|---|---|
| Number of subjects | 73 | 5 | 78 |
| Number of subjects without censoring [1] | 56 | 3 | 59 |
| Number of subjects with right censoring [1] | 17 | 2 | 19 |
| Mean (SD) | 120.3 (87.83) | 195.2 (130.61) | 125.1 (91.87) |
| Median | 112.0 | 280.0 | 112.0 |
| Minimum, Maximum | 1, 309 | 41, 309 | 1, 309 |

Duration of 2nd Success (days)

| | | | |
|---|---|---|---|
| Number of subjects | 32 | 2 | 34 |
| Number of subjects without censoring [1] | 22 | 1 | 23 |
| Number of subjects with right censoring [1] | 10 | 1 | 11 |
| Mean (SD) | 89.1 (67.03) | 63.5 (88.39) | 87.6 (67.05) |
| Median | 71.0 | 63.5 | 71.0 |
| Minimum, Maximum | 1, 257 | 1, 126 | 1, 257 |

Duration of 3rd Success (days)

| | | | |
|---|---|---|---|
| Number of subjects | 10 | 1 | 11 |
| Number of subjects without censoring [1] | 4 | 0 | 4 |
| Number of subjects with right censoring [1] | 6 | 1 | 7 |
| Mean (SD) | 36.2 (29.31) | 1.0 (NE) | 33.0 (29.76) |
| Median | 28.5 | 1.0 | 28.0 |
| Minimum, Maximum | 1, 85 | 1, 1 | 1, 85 |

Duration of Last Success for Each Subject (days)

| | | | |
|---|---|---|---|
| Number of subjects | 73 | 5 | 78 |
| Number of subjects without censoring [1] | 40 | 1 | 41 |
| Number of subjects with right censoring [1] | 33 | 4 | 37 |
| Mean (SD) | 118.0 (93.58) | 174.4 (158.72) | 121.6 (98.44) |
| Median | 112.0 | 280.0 | 112.0 |
| Minimum, Maximum | 1, 309 | 1, 309 | 1, 309 |

Average Duration of Treatment Success for Each Subject (days)

| | | | |
|---|---|---|---|
| Number of subjects | 73 | 5 | 78 |
| Mean (SD) | 119.7 (82.74) | 191.0 (136.91) | 124.2 (87.66) |
| Median | 98.0 | 280.0 | 98.0 |
| Minimum, Maximum | 1, 309 | 21, 309 | 1, 309 |

Total Duration of Treatment Success for Each Subject (days)

| | | | |
|---|---|---|---|
| Number of subjects | 73 | 5 | 78 |
| Mean (SD) | 164.3 (82.24) | 220.8 (109.21) | 167.9 (84.48) |
| Median | 168.0 | 280.0 | 170.0 |
| Minimum, Maximum | 1, 309 | 42, 309 | 1, 309 |

Baseline PGA = 1 (Almost Clear)

| | | |
|---|---|---|
| Tapinarof | Vehicle | |
| Cream, 1% | Cream | |
| (Pivotal) | (Pivotal) | Overall |
| (N = 73) | (N = 5) | (N = 78) |

Duration of 1st Success (days)

| | | | |
|---|---|---|---|
| Number of subjects | 76 | 9 | 85 |
| Number of subjects without censoring [1] | 49 | 5 | 54 |
| Number of subjects with right censoring [1] | 27 | 4 | 31 |
| Mean (SD) | 106.3 (77.57) | 115.0 (71.74) | 107.2 (76.62) |
| Median | 86.0 | 127.0 | 90.0 |
| Minimum, Maximum | 1, 280 | 1, 241 | 1, 280 |

Duration of 2nd Success (days)

| | | | |
|---|---|---|---|
| Number of subjects | 18 | 1 | 19 |
| Number of subjects without censoring [1] | 10 | 0 | 10 |
| Number of subjects with right censoring [1] | 8 | 1 | 9 |
| Mean (SD) | 71.6 (58.68) | 144.0 (NE) | 75.4 (59.40) |
| Median | 52.0 | 144.0 | 56.0 |
| Minimum, Maximum | 1, 189 | 144, 144 | 1, 189 |

Duration of 3rd Success (days)

| | | | |
|---|---|---|---|
| Number of subjects | 2 | 0 | 2 |
| Number of subjects with right censoring [1] | 2 | 0 | 2 |
| Mean (SD) | 15.0 (19.80) | — | 15.0 (19.80) |
| Median | 15.0 | — | 15.0 |
| Minimum, Maximum | 1, 29 | — | 1, 29 |

Duration of Last Success for Each Subject (days)

| | | | |
|---|---|---|---|
| Number of subjects | 76 | 9 | 85 |
| Number of subjects without censoring [1] | 39 | 4 | 43 |
| Number of subjects with right censoring [1] | 37 | 5 | 42 |
| Mean (SD) | 107.2 (79.80) | 118.9 (72.32) | 108.5 (78.72) |
| Median | 85.5 | 136.0 | 90.0 |
| Minimum, Maximum | 1, 280 | 1, 241 | 1, 280 |

Average Duration of Treatment Success for Each Subject (days)

| | | | |
|---|---|---|---|
| Number of subjects | 76 | 9 | 85 |
| Mean (SD) | 106.8 (76.36) | 116.9 (71.80) | 107.9 (75.54) |
| Median | 92.5 | 127.0 | 98.0 |
| Minimum, Maximum | 1, 280 | 1, 241 | 1, 280 |

Total Duration of Treatment Success for Each Subject (days)

| | | | |
|---|---|---|---|
| Number of subjects | 76 | 9 | 85 |
| Mean (SD) | 123.7 (78.29) | 131.0 (85.06) | 124.5 (78.53) |
| Median | 116.5 | 136.0 | 120.0 |
| Minimum, Maximum | 1, 280 | 1, 253 | 1, 280 |

Baseline PGA = 2 (Mild)

| | | |
|---|---|---|
| Tapinarof | Vehicle | |
| Cream, 1% | Cream | |
| (Pivotal) | (Pivotal) | Overall |
| (N = 188) | (N = 60) | (N = 248) |

Duration of 1st Success (days)

| | | | |
|---|---|---|---|
| Number of subjects | 49 | 22 | 71 |
| Number of subjects without censoring [1] | 29 | 13 | 42 |
| Number of subjects with right censoring [1] | 20 | 9 | 29 |
| Mean (SD) | 81.3 (71.03) | 110.8 (72.69) | 90.4 (72.34) |
| Median | 57.0 | 115.5 | 83.0 |
| Minimum, Maximum | 1, 281 | 1, 253 | 1, 281 |

Duration of 2nd Success (days)

| | | | |
|---|---|---|---|
| Number of subjects | 6 | 10 | 16 |
| Number of subjects without censoring [1] | 1 | 2 | 3 |
| Number of subjects with right censoring [1] | 5 | 8 | 13 |
| Mean (SD) | 27.3 (30.06) | 34.7 (51.14) | 31.9 (43.41) |
| Median | 25.0 | 27.5 | 27.5 |
| Minimum, Maximum | 1, 83 | 1, 170 | 1, 170 |

Duration of 3rd Success (days)

| | | | |
|---|---|---|---|
| Number of subjects | 0 | 1 | 1 |
| Number of subjects with right censoring [1] | 0 | 1 | 1 |
| Mean (SD) | — | 29.0 (NE) | 29.0 (NE) |
| Median | — | 29.0 | 29.0 |
| Minimum, Maximum | — | 29, 29 | 29, 29 |

Duration of Last Success for Each Subject (days)

| | | | |
|---|---|---|---|
| Number of subjects | 49 | 22 | 71 |
| Number of subjects without censoring [1] | 24 | 4 | 28 |
| Number of subjects with right censoring [1] | 25 | 18 | 43 |
| Mean (SD) | 76.1 (72.82) | 79.8 (77.74) | 77.3 (73.84) |
| Median | 56.0 | 44.5 | 56.0 |
| Minimum, Maximum | 1, 281 | 1, 253 | 1, 281 |

Average Duration of Treatment Success for Each Subject (days)

| | | | |
|---|---|---|---|
| Number of subjects | 49 | 22 | 71 |
| Mean (SD) | 78.7 (71.03) | 94.6 (65.57) | 83.6 (69.31) |
| Median | 57.0 | 87.0 | 66.5 |
| Minimum, Maximum | 1, 281 | 1, 253 | 1, 281 |

Total Duration of Treatment Success for Each Subject (days)

| | | | |
|---|---|---|---|
| Number of subjects | 49 | 22 | 71 |
| Mean (SD) | 84.6 (71.17) | 127.9 (73.01) | 98.0 (74.01) |
| Median | 78.0 | 136.5 | 84.0 |
| Minimum, Maximum | 1, 281 | 1, 253 | 1, 281 |

Baseline PGA = 3 (Moderate)

| | | |
|---|---|---|
| Tapinarof | Vehicle | |
| Cream, 1% | Cream | |
| (Pivotal) | (Pivotal) | Overall |
| (N = 93) | (N = 156) | (N = 249) |

Duration of 1st Success (days)

| | | | |
|---|---|---|---|
| Number of subjects | 12 | 49 | 61 |
| Number of subjects without censoring [1] | 6 | 24 | 30 |
| Number of subjects with right censoring [1] | 6 | 25 | 31 |
| Mean (SD) | 62.6 (55.19) | 70.3 (58.38) | 68.8 (57.40) |
| Median | 43.0 | 56.0 | 56.0 |
| Minimum, Maximum | 1, 171 | 1, 247 | 1, 247 |

Duration of 2nd Success (days)

| | | | |
|---|---|---|---|
| Number of subjects | 1 | 11 | 12 |
| Number of subjects without censoring [1] | 1 | 7 | 8 |
| Number of subjects with right censoring [1] | 0 | 4 | 4 |
| Mean (SD) | 57.0 (NE) | 29.2 (18.23) | 31.5 (19.15) |
| Median | 57.0 | 28.0 | 28.5 |
| Minimum, Maximum | 57, 57 | 1, 62 | 1, 62 |

Duration of 3rd Success (days)

| | | | |
|---|---|---|---|
| Number of subjects | 0 | 1 | 1 |
| Number of subjects with right censoring [1] | 0 | 1 | 1 |
| Mean (SD) | | 28.0 (NE) | 28.0 (NE) |
| Median | | 28.0 | 28.0 |
| Minimum, Maximum | | 28, 28 | 28, 28 |

Duration of Last Success for Each Subject (days)

| | | | |
|---|---|---|---|
| Number of subjects | 12 | 49 | 61 |
| Number of subjects without censoring [1] | 6 | 20 | 26 |
| Number of subjects with right censoring [1] | 6 | 29 | 35 |
| Mean (SD) | 65.2 (54.03) | 63.8 (59.74) | 64.1 (58.23) |
| Median | 56.0 | 49.0 | 51.0 |
| Minimum, Maximum | 1, 171 | 1, 247 | 1, 247 |

Average Duration of Treatment Success for Each Subject (days)

| | | | |
|---|---|---|---|
| Number of subjects | 12 | 49 | 61 |
| Mean (SD) | 63.9 (54.43) | 67.1 (57.99) | 66.4 (56.88) |
| Median | 48.3 | 51.0 | 51.0 |
| Minimum, Maximum | 1, 171 | 1, 247 | 1, 247 |

Total Duration of Treatment Success for Each Subject (days)

| | | | |
|---|---|---|---|
| Number of subjects | 12 | 49 | 61 |
| Mean (SD) | 67.3 (54.20) | 77.4 (58.26) | 75.5 (57.18) |
| Median | 56.5 | 81.0 | 64.0 |
| Minimum, Maximum | 1, 171 | 1, 247 | 1, 247 |

Baseline PGA = 4 (Severe)

| | | |
|---|---|---|
| Tapinarof | Vehicle | |
| Cream, 1% | Cream | |
| (Pivotal) | (Pivotal) | Overall |
| (N = 7) | (N = 16) | (N = 23) |

Duration of 1st Success (days)

| | | | |
|---|---|---|---|
| Number of subjects | 2 | 2 | 4 |
| Number of subjects without censoring [1] | 0 | 1 | 1 |
| Number of subjects with right censoring [1] | 2 | 1 | 3 |
| Mean (SD) | 119.5 (79.90) | 89.5 (47.38) | 104.5 (56.36) |
| Median | 119.5 | 89.5 | 93.0 |
| Minimum, Maximum | 63, 176 | 56, 123 | 56, 176 |

Duration of Last Success for Each Subject (days)

| | | | |
|---|---|---|---|
| Number of subjects | 2 | 2 | 4 |
| Number of subjects without censoring [1] | 0 | 1 | 1 |
| Number of subjects with right censoring [1] | 2 | 1 | 3 |
| Mean (SD) | 119.5 (79.90) | 89.5 (47.38) | 104.5 (56.36) |
| Median | 119.5 | 89.5 | 93.0 |
| Minimum, Maximum | 63, 176 | 56, 123 | 56, 176 |

Average Duration of Treatment Success for Each Subject (days)

| | | | |
|---|---|---|---|
| Number of subjects | 2 | 2 | 4 |
| Mean (SD) | 119.5 (79.90) | 89.5 (47.38) | 104.5 (56.36) |
| Median | 119.5 | 89.5 | 93.0 |
| Minimum, Maximum | 63, 176 | 56, 123 | 56, 176 |

Total Duration of Treatment Success for Each Subject (days)

| | | | |
|---|---|---|---|
| Number of subjects | 2 | 2 | 4 |
| Mean (SD) | 119.5 (79.90) | 89.5 (47.38) | 104.5 (56.36) |
| Median | 119.5 | 89.5 | 93.0 |
| Minimum, Maximum | 63, 176 | 56, 123 | 56, 176 |

[1] Right censoring indicates that treatment success was ongoing at the end of the study.
DB = double blinded;
ITT = Intent-to-Treat;
NE = not estimable;
PGA = Physician Global Assessment;
SD = standard deviation.

Physician Global Assessment Score, Percent of Body Surface Area Affected, and Psoriasis Area and Severity Index

Physician Global Assessment

Table 12 presents PGA score and change from baseline at Week 16 and Week 40 as performed using OC and LOCF methods. Overall subjects' baseline PGA scores were similarly distributed among moderate (32.8%), mild (32.7%), and almost clear (21.2%). The majority of subjects who were randomized to vehicle cream in the DB, pivotal Phase 3 studies entered this extension study with a Baseline PGA=3 (61.4%) compared with 18.4% of subjects randomized to tapinarof cream, 1%. The majority of subjects randomized to tapinarof cream, 1% in the DB, pivotal Phase 3 studies entered with a PGA score of either a 2 (37.2%) or 1 (28.5%).

At Week 16 using the OC method, 19.1% and 32.2% of subjects randomized to tapinarof cream, 1% in the DB, pivotal Phase 3 studies had a PGA=0 or PGA=1, respectively; and 12.9% and 28.2% of subjects randomized to vehicle cream had a PGA=0 or PGA=1, respectively. Using the LOCF method, 18.1% and 30.4% of subjects randomized to tapinarof cream, 1% in the DB, pivotal Phase 3 studies had a PGA=0 or PGA=1, respectively; and 12.7% and 25.8% of subjects randomized to vehicle cream had a PGA=0 or PGA=1.

At Week 40 using the OC method, 17.4% and 30.1% of subjects randomized to tapinarof cream, 1% in the DB, pivotal Phase 3 studies had a PGA=0 or PGA=1, respectively; and 23.9% and 31.9% of subjects randomized to vehicle cream had a PGA=0 or PGA=1, respectively. Using the LOCF method, 15.4% and 30.0% of subjects randomized to tapinarof cream, 1% in the DB, pivotal Phase 3 studies had a PGA=0 or PGA=1, respectively; and 16.7% and 27.4% of subjects randomized to vehicle cream had a PGA=0 or PGA=1, respectively.

TABLE 12
Summary of PGA Scores Observed Values and Change from Baseline at
Week 16 and Week 40 (ITT Population, Observed Cases and Last
Observation Carried Forward)

| | | |
|---|---|---|
| Tapinarof | Vehicle | |
| Cream, 1% | Cream | |
| (Pivotal) | (Pivotal) | Overall |
| (N = 508) | (N = 255) | (N = 763) |

Baseline
Observed Value, n (%)

| | | | |
|---|---|---|---|
| N | 505 | 254 | 759 |
| 4 - Severe | 7 (1.4) | 16 (6.3) | 23 (3.0) |
| 3 - Moderate | 93 (18.4) | 156 (61.4) | 249 (32.8) |
| 2 - Mild | 188 (37.2) | 60 (23.6) | 248 (32.7) |
| 1 - Almost Clear | 144 (28.5) | 17 (6.7) | 161 (21.2) |
| 0 - Clear | 73 (14.5) | 5 (2.0) | 78 (10.3) |

Week 16, Observed Cases
Observed Value, n (%)

| | | | |
|---|---|---|---|
| N | 419 | 209 | 628 |
| 4 - Severe | 4 (1.0) | 1 (0.5) | 5 (0.8) |
| 3 - Moderate | 54 (12.9) | 32 (15.3) | 86 (13.7) |
| 2 - Mild | 146 (34.8) | 90 (43.1) | 236 (37.6) |
| 1 - Almost Clear | 135 (32.2) | 59 (28.2) | 194 (30.9) |
| 0 - Clear | 80 (19.1) | 27 (12.9) | 107 (17.0) |

Change from Baseline, n (%)

| | | | |
|---|---|---|---|
| N | 418 | 209 | 627 |
| +4 = Worsened by 4 | 0 (0.0) | 0 (0.0) | 0 (0.0) |
| +3 = Worsened by 3 | 5 (1.2) | 0 (0.0) | 5 (0.8) |
| +2 = Worsened by 2 | 23 (5.5) | 2 (1.0) | 25 (4.0) |
| +1 = Worsened by 1 | 63 (15.1) | 6 (2.9) | 69 (11.0) |
| 0 = No Change | 187 (44.7) | 59 (28.2) | 246 (39.2) |
| −1 = Improved by 1 | 109 (26.1) | 81 (38.8) | 190 (30.3) |
| −2 = Improved by 2 | 29 (6.9) | 48 (23.0) | 77 (12.3) |
| −3 = Improved by 3 | 1 (0.2) | 12 (5.7) | 13 (2.1) |
| −4 = Improved by 4 | 1 (0.2) | 1 (0.5) | 2 (0.3) |

Week 16, Last Observation Carried Forward
Observed Value, n (%)

| | | | |
|---|---|---|---|
| N | 493 | 252 | 745 |
| 4 - Severe | 6 (1.2) | 5 (2.0) | 11 (1.5) |
| 3 - Moderate | 74 (15.0) | 45 (17.9) | 119 (16.0) |
| 2 - Mild | 174 (35.3) | 105 (41.7) | 279 (37.4) |
| 1 - Almost Clear | 150 (30.4) | 65 (25.8) | 215 (28.9) |
| 0 - Clear | 89 (18.1) | 32 (12.7) | 121 (16.2) |

Change from Baseline, n (%)

| | | | |
|---|---|---|---|
| N | 491 | 251 | 742 |
| +4 = Worsened by 4 | 0 (0.0) | 0 (0.0) | 0 (0.0) |
| +3 = Worsened by 3 | 5 (1.0) | 0 (0.0) | 5 (0.7) |
| +2 = Worsened by 2 | 25 (5.1) | 3 (1.2) | 28 (3.8) |
| +1 = Worsened by 1 | 77 (15.7) | 9 (3.6) | 86 (11.6) |
| 0 = No Change | 223 (45.4) | 74 (29.5) | 297 (40.0) |
| −1 = Improved by 1 | 122 (24.8) | 97 (38.6) | 219 (29.5) |
| −2 = Improved by 2 | 37 (7.5) | 54 (21.5) | 91 (12.3) |
| −3 = Improved by 3 | 1 (0.2) | 13 (5.2) | 14 (1.9) |
| −4 = Improved by 4 | 1 (0.2) | 1 (0.4) | 2 (0.3) |

Week 40, Observed Cases
Observed Value, n (%)

| | | | |
|---|---|---|---|
| N | 236 | 113 | 349 |
| 4 - Severe | 1 (0.4) | 0 (0.0) | 1 (0.3) |
| 3 - Moderate | 42 (17.8) | 15 (13.3) | 57 (16.3) |
| 2 - Mild | 81 (34.3) | 35 (31.0) | 116 (33.2) |
| 1 - Almost Clear | 71 (30.1) | 36 (31.9) | 107 (30.7) |
| 0 - Clear | 41 (17.4) | 27 (23.9) | 68 (19.5) |

Change from Baseline, n (%)

| | | | |
|---|---|---|---|
| N | 236 | 113 | 349 |
| +4 = Worsened by 4 | 0 (0.0) | 0 (0.0) | 0 (0.0) |
| +3 = Worsened by 3 | 3 (1.3) | 0 (0.0) | 3 (0.9) |
| +2 = Worsened by 2 | 20 (8.5) | 1 (0.9) | 21 (6.0) |
| +1 = Worsened by 1 | 48 (20.3) | 7 (6.2) | 55 (15.8) |
| 0 = No Change | 86 (36.4) | 19 (16.8) | 105 (30.1) |
| −1 = Improved by 1 | 58 (24.6) | 41 (36.3) | 99 (28.4) |
| −2 = Improved by 2 | 19 (8.1) | 27 (23.9) | 46 (13.2) |
| −3 = Improved by 3 | 2 (0.8) | 17 (15.0) | 19 (5.4) |
| −4 = Improved by 4 | 0 (0.0) | 1 (0.9) | 1 (0.3) |

Week 40, Last Observation Carried Forward
Observed Value, n (%)

| | | | |
|---|---|---|---|
| N | 493 | 252 | 745 |
| 4 - Severe | 7 (1.4) | 4 (1.6) | 11 (1.5) |
| 3 - Moderate | 91 (18.5) | 55 (21.8) | 146 (19.6) |
| 2 - Mild | 171 (34.7) | 82 (32.5) | 253 (34.0) |
| 1 - Almost Clear | 148 (30.0) | 69 (27.4) | 217 (29.1) |
| 0 - Clear | 76 (15.4) | 42 (16.7) | 118 (15.8) |

Change from Baseline, n (%)

| | | | |
|---|---|---|---|
| N | 491 | 251 | 742 |
| +4 = Worsened by 4 | 0 (0.0) | 0 (0.0) | 0 (0.0) |
| +3 = Worsened by 3 | 4 (0.8) | 0 (0.0) | 4 (0.5) |
| +2 = Worsened by 2 | 34 (6.9) | 3 (1.2) | 37 (5.0) |
| +1 = Worsened by 1 | 95 (19.3) | 13 (5.2) | 108 (14.6) |
| 0 = No Change | 211 (43.0) | 71 (28.3) | 282 (38.0) |
| −1 = Improved by 1 | 110 (22.4) | 84 (33.5) | 194 (26.1) |
| −2 = Improved by 2 | 31 (6.3) | 57 (22.7) | 88 (11.9) |
| −3 = Improved by 3 | 5 (1.0) | 22 (8.8) | 27 (3.6) |
| −4 = Improved by 4 | 1 (0.2) | 1 (0.4) | 2 (0.3) |

DB = double blinded;
ITT = Intent-to-Treat;
PGA = Physician Global Assessment.

Body Surface Area

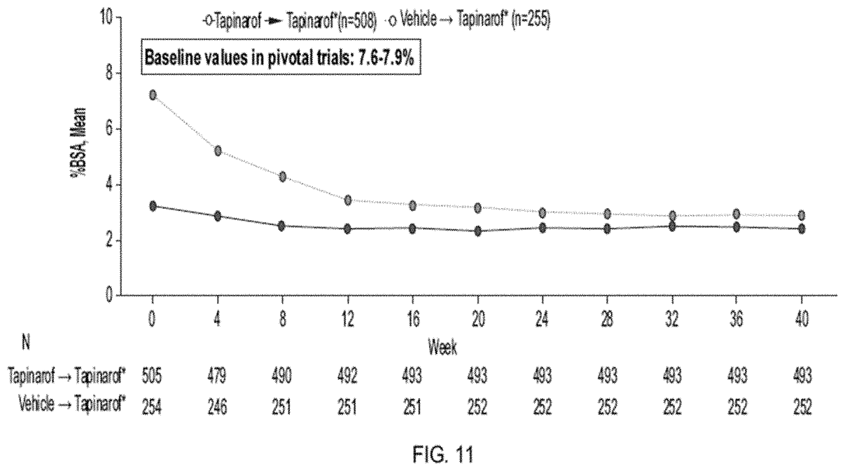

Table 13 presents % BSA affected, change from baseline, and percent change from Baseline at Week 16 and Week 40 as performed using OC and LOCF methods. FIG. 11 shows the change in % BSA affected throughout the duration of the trial. Overall subjects' Baseline mean (SD) % BSA affected was 4.65% (5.601) with 3.31% (4.743) for subjects who were randomized to tapinarof cream 1% in the DB, pivotal Phase 3 studies and 7.30% (6.208) for subjects who were randomized to vehicle cream.

The % BSA affected decreased over time overall and in both DB assigned treatment groups with no loss of effect observed. At Week 16 using the OC method, the mean (SD) % BSA affected was 1.97% (2.694) for subjects randomized to tapinarof cream, 1% in the DB, pivotal Phase 3 studies and 2.89% (4.028) for subjects randomized to vehicle cream. Overall, the mean (SD) % BSA affected was 2.28% (3.226). Using the LOCF method resulted in mean (SD) % BSA affected of 2.54% (4.579) and 3.36% (4.546), for subjects randomized to tapinarof cream, 1% cream or vehicle cream in the DB, pivotal Phase 3 studies, respectively. The overall mean (SD) % BSA affected was 2.82% (4.581).

At Week 40 using the OC method, the mean (SD) % BSA affected was 1.75% (2.714) for subjects randomized to tapinarof cream, 1% in the DB, pivotal Phase 3 studies and 2.07% (3.703) for subjects randomized to vehicle cream. Overall, the mean (SD) % BSA affected was 1.85% (3.067). Using the LOCF method resulted in mean (SD) % BSA affected of 2.44% (4.645) and 2.99% (4.472) for subjects randomized to tapinarof cream, 1% cream or vehicle cream in the DB, pivotal Phase 3 studies, respectively. The overall mean (SD) % BSA affected was 2.63% (4.592).

TABLE 13
Summary of Percent BSA Affected Observed Values, Change from
Baseline, and Percent Change from Baseline at Week 16 and Week 40 (ITT Population,
Observed Cases and Last Observation Carried Forward)

| Tapinarof Cream 1% | | | Vehicle Cream | | | | | |
|---|---|---|---|---|---|---|---|---|
| (Pivotal) | | | (Pivotal) | | | Overall | | |
| (N = 508) | | | (N = 255) | | | (N = 763) | | |
| | | Percent | | | Percent | | | Percent |
| Observed | CFBL | CFBL | Observed | CFBL | CFBL | Observed | CFBL | CFBL |

Baseline

| | | | |
|---|---|---|---|
| N | 505 | 254 | 759 |
| Mean (SD) | 3.31 | 7.30 | 4.65 |
| | (4.743) | (6.208) | (5.601) |
| Median | 2.00 | 5.65 | 3.00 |
| Minimum, | 0.0, 66.0 | 0.0, 45.0 | 0.0, 66.0 |
| Maximum | | | |

Week 16, Observed Cases

| | | | | | | | | | |
|---|---|---|---|---|---|---|---|---|---|
| N | 419 | 418 | 350 | 209 | 209 | 204 | 628 | 627 | 554 |
| Mean (SD) | 1.97 | −0.97 | −18.46 | 2.89 | −4.40 | −53.18 | 2.28 | −2.11 | −31.25 |
| | (2.694) | (3.090) | (86.088) | (4.028) | (5.043) | (77.450) | (3.226) | (4.175) | (84.617) |
| Median | 1.00 | −0.23 | −36.96 | 1.50 | −3.18 | −66.67 | 1.02 | −1.00 | −50.00 |
| Minimum, | 0.0, 20.8 | −27.1, | −100.0, | 0.0, 22.0 | −27.1, | −100.0, | 0.0, 22.0 | −27.1, | −100.0, |
| Maximum | | 10.5 | 525.0 | | 14.3 | 841.2 | | 14.3 | 841.2 |

Week 16, Last Observation Carried Forward

| | | | | | | | | | |
|---|---|---|---|---|---|---|---|---|---|
| N | 493 | 491 | 420 | 252 | 251 | 246 | 745 | 742 | 666 |
| Mean (SD) | 2.54 | −0.77 | −5.67 | 3.36 | −3.96 | −47.15 | 2.82 | −1.85 | −20.99 |
| | (4.579) | (3.330) | (150.062) | (4.546) | (5.028) | (81.350) | (4.581) | (4.259) | (130.491) |
| Median | 1.00 | −0.20 | −33.33 | 1.80 | −2.98 | −63.42 | 1.20 | −0.96 | −46.13 |
| Minimum, | 0.0, 66.0 | −27.1, | −100.0, | 0.0, 26.2 | −27.1, | −100.0, | 0.0, 66.0 | −27.1, | −100.0, |
| Maximum | | 17.2 | 2278.6 | | 14.3 | 841.2 | | 17.2 | 2278.6 |

Week 40, Observed Cases

| | | | | | | | | | |
|---|---|---|---|---|---|---|---|---|---|
| N | 236 | 236 | 197 | 113 | 113 | 110 | 349 | 349 | 307 |
| Mean (SD) | 1.75 | −0.93 | −21.60 | 2.07 | −5.30 | −60.22 | 1.85 | −2.35 | −35.43 |
| | (2.714) | (3.100) | (126.790) | (3.703) | (5.594) | (102.651) | (3.067) | (4.556) | (119.977) |
| Median | 0.68 | −0.49 | −57.39 | 0.80 | −4.49 | −86.06 | 0.72 | −1.50 | −67.00 |
| Minimum, | 0.0, 17.6 | −17.8, | −100.0, | 0.0, 23.0 | −29.0, | −100.0, | 0.0, 23.0 | −29.0, | −100.0, |
| Maximum | | 12.6 | 1194.3 | | 10.8 | 860.0 | | 12.6 | 1194.3 |

Week 40, Last Observation Carried Forward

| | | | | | | | | | |
|---|---|---|---|---|---|---|---|---|---|
| N | 493 | 491 | 420 | 252 | 251 | 246 | 745 | 742 | 666 |
| Mean (SD) | 2.44 | −0.88 | −0.11 | 2.99 | −4.33 | −43.91 | 2.63 | −2.04 | −16.29 |
| | (4.645) | (3.834) | (183.058) | (4.472) | (5.702) | (109.158) | (4.592) | (4.833) | (161.095) |
| Median | 1.00 | −0.30 | −46.61 | 1.00 | −3.30 | −75.06 | 1.00 | −1.00 | −55.78 |
| Minimum, | 0.0, 66.0 | −27.1, | −100.0, | 0.0, 26.2 | −29.0, | −100.0, | 0.0, 66.0 | −29.0, | −100.0, |
| Maximum | | 17.2 | 2278.6 | | 14.3 | 860.0 | | 17.2 | 2278.6 |

BSA = body surface area;
CFBL = change from baseline;
DB = double blinded;
ITT = Intent-to-Treat;
PGA = Physician Global Assessment;
SD = standard deviation.

Psoriasis Area and Severity Index

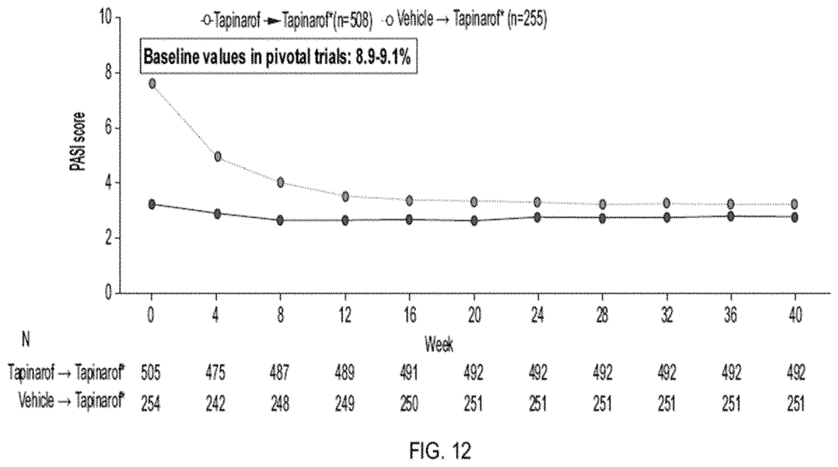

Table 14 presents observed PASI score, change from baseline, and percent change from baseline at Week 16 and Week 40 as performed using OC and LOCF methods. FIG. 12 shows the change in PASI score throughout the duration of the trial. Overall subjects' Baseline mean (SD) PASI score was 4.76 (4.724) with 3.28 (3.531) for subjects who were randomized to tapinarof cream, 1% in the DB, pivotal Phase 3 studies and 7.69 (5.387) for subjects who were randomized to vehicle cream.

PASI score decreased over time overall and in both DB assigned treatment groups with no loss of effect observed. At Week 16 using the OC method, the mean (SD) PASI score was 2.38 (2.652) for subjects randomized to tapinarof cream, 1% in the DB, pivotal Phase 3 studies and 2.95 (3.020) for subjects randomized to vehicle cream. Overall, the mean (SD) PASI score was 2.57 (2.791). Using the LOCF method resulted in mean (SD) PASI score of 2.74 (3.324) and 3.47 (3.794) for subjects randomized to tapinarof cream, 1% cream or vehicle cream in the DB, pivotal Phase 3 studies, respectively. The overall mean (SD) PASI was 2.99 (3.504).

At Week 40 using the OC method, the mean (SD) PASI score was 2.38 (2.672) for subjects randomized to tapinarof cream, 1% in the DB, pivotal Phase 3 studies and 2.31 (3.008) for subjects randomized to vehicle cream. Overall, the mean (SD) PASI score was 2.36 (2.781). Using the LOCF method resulted in mean (SD) PASI score of 2.80 (3.443) and 3.28 (3.937) for subjects randomized to tapinarof cream, 1% cream or vehicle cream in the DB, pivotal Phase 3 studies, respectively. The overall mean (SD) PASI score was 2.97 (3.622).

TABLE 14
Summary of PASI Observed Values, Change from Baseline, and Percent
Change from Baseline at Week 16 and Week 40 (ITT Population,
Observed Cases and Last Observation Carried Forward)

| Tapinarof Cream 1% | | | Vehicle Cream | | | | | |
|---|---|---|---|---|---|---|---|---|
| (Pivotal) | | | (Pivotal) | | | Overall | | |
| (N = 508) | | | (N = 255) | | | (N = 763) | | |
| | | Percent | | | Percent | | | Percent |
| Observed | CFBL | CFBL | Observed | CFBL | CFBL | Observed | CFBL | CFBL |

Baseline

| | | | |
|---|---|---|---|
| N | 505 | 254 | 759 |
| Mean (SD) | 3.28 | 7.69 | 4.76 |
| | (3.531) | (5.387) | (4.724) |
| Median | 2.30 | 6.40 | 3.40 |
| Minimum, | 0.0, 28.8 | 0.0, 28.2 | 0.0, 28.8 |
| Maximum | | | |

Week 16, Observed Cases

| | | | | | | | | | |
|---|---|---|---|---|---|---|---|---|---|
| N | 412 | 411 | 342 | 207 | 207 | 202 | 619 | 618 | 544 |
| Mean (SD) | 2.38 | −0.67 | −7.79 | 2.95 | −4.59 | −55.09 | 2.57 | −1.99 | −25.35 |
| | (2.652) | (2.888) | (106.357) | (3.020) | (4.294) | (47.486) | (2.791) | (3.889) | (91.988) |
| Median | 1.60 | −0.40 | −30.00 | 2.10 | −3.80 | −64.02 | 1.80 | −1.00 | −44.72 |
| Minimum, | 0.0, 18.0 | −20.4, | −100.0, | 0.0, 16.2 | −18.2, | −100.0, | 0.0, 18.0 | −20.4, | −100.0, |
| Maximum | | 10.8 | 600.0 | | 9.7 | 323.3 | | 10.8 | 600.0 |

Week 16, Last Observation Carried Forward

| | | | | | | | | | |
|---|---|---|---|---|---|---|---|---|---|
| N | 491 | 489 | 416 | 250 | 250 | 245 | 741 | 739 | 661 |
| Mean (SD) | 2.74 | −0.54 | −3.20 | 3.47 | −4.24 | −51.02 | 2.99 | −1.80 | −20.93 |
| | (3.324) | (2.930) | (106.527) | (3.794) | (4.221) | (48.996) | (3.504) | (3.841) | (92.505) |
| Median | 1.80 | −0.20 | −25.00 | 2.40 | −3.80 | −59.38 | 1.80 | −0.90 | −40.91 |
| Minimum, | 0.0, 28.8 | −20.4, | −100.0, | 0.0, 23.5 | −18.2, | −100.0, | 0.0, 28.8 | −20.4, | −100.0, |
| Maximum | | 14.0 | 600.0 | | 9.7 | 323.3 | | 14.0 | 600.0 |

Week 40, Observed Cases

| | | | | | | | | | |
|---|---|---|---|---|---|---|---|---|---|
| N | 236 | 236 | 196 | 113 | 113 | 110 | 349 | 349 | 306 |
| Mean (SD) | 2.38 | −0.51 | 8.49 | 2.31 | −5.53 | −60.12 | 2.36 | −2.14 | −16.18 |
| | (2.672) | (3.189) | (222.467) | (3.008) | (4.957) | (62.520) | (2.781) | (4.506) | (184.734) |
| Median | 1.60 | −0.30 | −35.24 | 1.40 | −4.80 | −76.39 | 1.60 | −1.20 | −56.70 |
| Minimum, | 0.0, 18.5 | −14.7, | −100.0, | 0.0, 17.5 | −28.2, | −100.0, | 0.0, 18.5 | −28.2, | −100.0, |
| Maximum | | 14.4 | 2700.0 | | 6.4 | 300.0 | | 14.4 | 600.0 |

Week 40, Last Observation Carried Forward

| | | | | | | | | | |
|---|---|---|---|---|---|---|---|---|---|
| N | 492 | 490 | 417 | 251 | 251 | 246 | 743 | 741 | 663 |
| Mean (SD) | 2.80 | −0.48 | 11.96 | 3.28 | −4.42 | −48.42 | 2.97 | −1.81 | −10.45 |
| | (3.443) | (3.360) | (184.055) | (3.937) | (4.814) | (68.495) | (3.622) | (4.332) | (154.519) |
| Median | 1.80 | −0.20 | −27.27 | 2.00 | −3.90 | −68.66 | 1.80 | −0.90 | −44.44 |
| Minimum, | 0.0, 28.8 | −20.4, | −100.0, | 0.0, 23.5 | −28.2, | −100.0, | 0.0, 28.8 | −28.2, | −100.0, |
| Maximum | | 14.4 | 2700.0 | | 9.7 | 481.6 | | 14.4 | 2700.0 |

CFBL = change from baseline;
DB = double blinded;
ITT = Intent-to-Treat;
PASI = Psoriasis Area and Severity Index;
PGA = Physician Global Assessment;
SD = standard deviation.

Table 15 presents subjects who achieved a reduction of ≥75% from Baseline in the PASI (PASI75) at Week 16 and Week 40 as performed using OC and LOCF methods.

There was continued efficacy in subjects randomized to tapinarof cream, 1% in the DB, pivotal Phase 3 studies and a greater percentage of subjects with reductions of ≥75% from Baseline in those randomized to vehicle cream, as expected based on the Baseline differences of the 2 DB assigned treatment groups. At Week 16 using the OC method, 141 subjects (22.8%) achieved a reduction of ≥75% from Baseline in their PASI score: 17.0% of subjects randomized to tapinarof cream, 1% in the DB, pivotal Phase 3 studies and 34.3% of subjects randomized to vehicle cream. Using the LOCF method, 158 subjects (21.3%) achieved a reduction of ≥75% from Baseline in their PASI score: 15.9% of subjects randomized to tapinarof cream, 1% in the DB, pivotal Phase 3 studies and 32.0% of subjects randomized to vehicle cream.

At Week 40 using the OC method, 108 subjects (30.9%) achieved a reduction of ≥75% from Baseline in their PASI score: 21.2% of subjects randomized to tapinarof cream, 1% in the DB, pivotal Phase 3 studies and 51.3% of subjects randomized to vehicle cream. Using the LOCF, 188 subjects (25.3%) achieved a reduction of ≥75% from Baseline in their PASI score: 16.9% of subjects randomized to tapinarof cream, 1% in the DB, pivotal Phase 3 studies and 41.8% of subjects randomized to vehicle cream.

TABLE 15
Summary of PASI75 Response Rate at Week 16 and Week 40
(ITT Population, Observed Cases and Last Observation Carried Forward)

| | | |
|---|---|---|
| Tapinarof | Vehicle | |
| Cream, 1% | Cream | |
| (Pivotal) | (Pivotal) | Overall |
| (N = 508) | (N = 255) | (N = 763) |

Week 16, Observed Cases

| | | | |
|---|---|---|---|
| PASI75 Responder [1], n/N (%) | 70/412 (17.0) | 71/207 (34.3) | 141/619 (22.8) |
| Standard error of percentage responder | 1.85 | 3.30 | 1.69 |
| PASI75 Non-Responder [1], n/N (%) | 342/412 (83.0) | 136/207 (65.7) | 478/619 (77.2) |
| Standard error of percentage non-responder | 1.85 | 3.30 | 1.69 |

Week 16, Last Observation Carried Forward

| | | | |
|---|---|---|---|
| PASI75 Responder [1], n/N (%) | 78/491 (15.9) | 80/250 (32.0) | 158/741 (21.3) |
| Standard error of percentage responder | 1.65 | 2.95 | 1.50 |
| PASI75 Non-Responder [1], n/N (%) | 413/491 (84.1) | 170/250 (68.0) | 583/741 (78.7) |
| Standard error of percentage non-responder | 1.65 | 2.95 | 1.50 |

Week 40, Observed Cases

| | | | |
|---|---|---|---|
| PASI75 Responder [1], n/N (%) | 50/236 (21.2) | 58/113 (51.3) | 108/349 (30.9) |
| Standard error of percentage responder | 2.66 | 4.70 | 2.47 |
| PASI75 Non-Responder [1], n/N (%) | 186/236 (78.8) | 55/113 (48.7) | 241/349 (69.1) |
| Standard error of percentage non-responder | 2.66 | 4.70 | 2.47 |

Week 40, Last Observation Carried Forward

| | | | |
|---|---|---|---|
| PASI75 Responder [1], n/N (%) | 83/492 (16.9) | 105/251 (41.8) | 188/743 (25.3) |
| Standard error of percentage responder | 1.69 | 3.11 | 1.59 |
| PASI75 Non-Responder [1], n/N (%) | 409/492 (83.1) | 146/251 (58.2) | 555/743 (74.7) |
| Standard error of percentage non-responder | 1.69 | 3.11 | 1.59 |

[1] Response defined as achieving a reduction of 75% or greater from Baseline in the PASI. Missing cases were imputed by last observation carried forward.
DB = double blinded;
ITT = Intent-to-Treat;
PASI = Psoriasis Area and Severity Index;
PASI75 = 75% improvement in PASI score;
PGA = Physician Global Assessment.

Table 16 presents subjects who achieved a reduction of ≥90% from Baseline in the PASI (PASI90) at Week 16 and Week 40 as performed using OC and LOCF methods.

There was continued efficacy in subjects randomized to tapinarof cream, 1% in the DB, pivotal Phase 3 studies and a greater percentage of subjects with reductions of ≥90% from Baseline in those randomized to vehicle cream, as expected based on the Baseline differences of the 2 DB assigned treatment groups. At Week 16 using the OC method, 89 subjects (14.4%) achieved a reduction of ≥90% from Baseline in their PASI score: 13.6% of subjects randomized to tapinarof cream, 1% in the DB, pivotal Phase 3 studies and 15.9% of subjects randomized to vehicle cream. Using the LOCF method, 102 subjects (13.8%) achieved a reduction of ≥90% from Baseline in their PASI score: 13.0% of subjects randomized to tapinarof cream, 1% in the DB, pivotal Phase 3 studies and 15.2% of subjects randomized to vehicle cream.

At Week 40 using the OC method, 62 subjects (17.8%) achieved a reduction of ≥90% from Baseline in their PASI score: 13.1% of subjects randomized to tapinarof cream, 1% in the DB, pivotal Phase 3 studies and 27.4% of subjects randomized to vehicle cream. Using the LOCF method, 107 subjects (14.4%) achieved a reduction of ≥90% from Baseline in their PASI score: 11.2% of subjects randomized to tapinarof cream, 1% in the DB, pivotal Phase 3 studies and 20.7% of subjects randomized to vehicle cream.

TABLE 16
Summary of PASI90 Response Rate at Week 16 and Week 40
(ITT Population, Observed Cases and Last Observation Carried
Forward)

| | | |
|---|---|---|
| Tapinarof | Vehicle | |
| Cream, 1% | Cream | |
| (Pivotal) | (Pivotal) | Overall |
| (N = 508) | (N = 255) | (N = 763) |

Week 16, Observed Cases

| | | | |
|---|---|---|---|
| PASI90 Responder [1], n/N (%) | 56/412 (13.6) | 33/207 (15.9) | 89/619 (14.4) |
| Standard error of percentage responder | 1.69 | 2.54 | 1.41 |
| PASI90 Non-Responder [1], n/N (%) | 356/412 (86.4) | 174/207 (84.1) | 530/619 (85.6) |
| Standard error of percentage non- | 1.69 | 2.54 | 1.41 |
| responder | | | |

Week 16, Last Observation Carried Forward

| | | | |
|---|---|---|---|
| PASI90 Responder [1], n/N (%) | 64/491 (13.0) | 38/250 (15.2) | 102/741 (13.8) |
| Standard error of percentage responder | 1.52 | 2.27 | 1.27 |
| PASI90 Non-Responder [1], n/N (%) | 427/491 (87.0) | 212/250 (84.8) | 639/741 (86.2) |
| Standard error of percentage non- | 1.52 | 2.27 | 1.27 |
| responder | | | |

Week 40, Observed Cases

| | | | |
|---|---|---|---|
| PASI90 Responder [1], n/N (%) | 31/236 (13.1) | 31/113 (27.4) | 62/349 (17.8) |
| Standard error of percentage responder | 2.20 | 4.20 | 2.05 |
| PASI90 Non-Responder [1], n/N (%) | 205/236 (86.9) | 82/113 (72.6) | 287/349 (82.2) |
| Standard error of percentage non- | 2.20 | 4.20 | 2.05 |
| responder | | | |

Week 40, Last Observation Carried Forward

| | | | |
|---|---|---|---|
| PASI90 Responder [1], n/N (%) | 55/492 (11.2) | 52/251 (20.7) | 107/743 (14.4) |
| Standard error of percentage responder | 1.42 | 2.56 | 1.29 |
| PASI90 Non-Responder [1], n/N (%) | 437/492 (88.8) | 199/251 (79.3) | 636/743 (85.6) |
| Standard error of percentage non- | 1.42 | 2.56 | 1.29 |
| responder | | | |

[1] Response defined as achieving a reduction of 90% or greater from Baseline in the PASI. Missing cases imputed by last observation carried forward.
DB = double blinded;
ITT = Intent-to-Treat;
PASI = Psoriasis Area and Severity Index;
PASI90 = 90% improvement in PASI score;
PGA = Physician Global Assessment.

Health Related Quality of Life Endpoint, Dermatology Life Quality Index

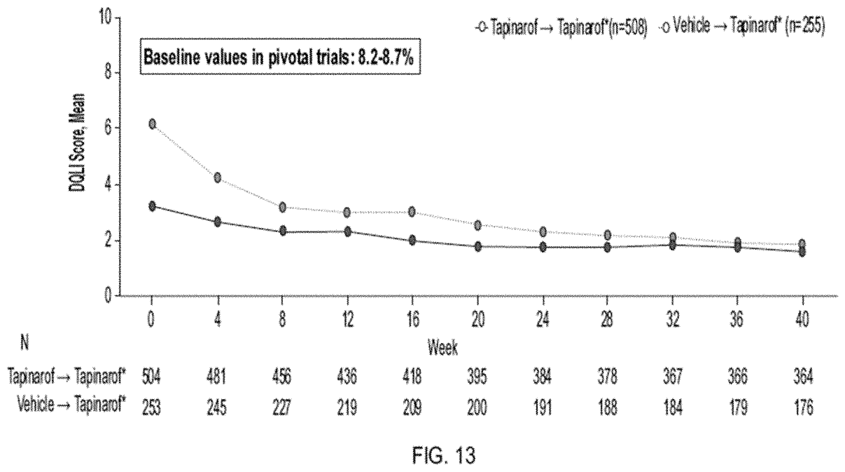

Table 17 presents the DLQI total score observed values, change from baseline, and percent change from baseline for Week 16 and Week 40. FIG. 13 shows the change in mean DQLI score throughout the duration of the trial. Overall subjects' Baseline mean (SD) DLQI total score was 4.3 (5.11) with 3.3 (4.33) for subjects who were randomized to tapinarof cream 1% in the DB, pivotal Phase 3 studies and 6.2 (5.97) for subjects who were randomized to vehicle cream.

DLQI scores decreased in both DB assigned treatment groups during this extension study with greater decreases in those subjects randomized to vehicle cream in the DB, pivotal Phase 3 studies, as expected based on the Baseline differences of the 2 DB assigned treatment groups. At Week 16 using the OC method, the mean (SD) DLQI total score was 2.0 (3.23) for subjects randomized to tapinarof cream, 1% in the DB, pivotal Phase 3 studies and 3.0 (5.19) for subjects randomized to vehicle cream. Overall, the mean (SD) DLQI total score was 2.3 (4.02).

By Week 40 using the OC method, the overall score and the scores from each DB assigned treatment group were ≤2.0. The Week 40 mean (SD) DLQI total score was 1.6 (2.36) for subjects randomized to tapinarof cream, 1% in the DB, pivotal Phase 3 studies and 2.0 (4.23) for subjects randomized to vehicle cream. Overall, the mean (SD) DLQI total score was 1.7 (3.09).

TABLE 17
Summary of Health Related Quality of Life Endpoint, DLQI Total Score
Observed Values, Change from Baseline, and Percent Change from
Baseline at Week 16 and Week 40 (ITT Population, Observed Cases)

| Tapinarof Cream, 1% | | | Vehicle Cream | | | | | |
|---|---|---|---|---|---|---|---|---|
| (Pivotal) | | | (Pivotal) | | | Overall | | |
| (N = 508) | | | (N = 255) | | | (N = 763) | | |
| | | Percent | | | Percent | | | Percent |
| Observed | CFBL | CFBL | Observed | CFBL | CFBL | Observed | CFBL | CFBL |

Baseline

| | | | |
|---|---|---|---|
| N | 504 | 253 | 757 |
| Mean (SD) | 3.3 | 6.2 | 4.3 |
| | (4.33) | (5.97) | (5.11) |
| Median | 2.0 | 4.0 | 2.0 |
| Minimum, | 0, 26 | 0, 30 | 0, 30 |
| Maximum | | | |

Week 16

| | | | | | | | | | |
|---|---|---|---|---|---|---|---|---|---|
| N | 418 | 416 | 307 | 209 | 208 | 188 | 627 | 624 | 495 |
| Mean (SD) | 2.0 | −1.0 | −21.3 | 3.0 | −3.1 | −45.9 | 2.3 | −1.7 | −30.7 |
| | (3.23) | (3.83) | (115.97) | (5.19) | (5.56) | (102.00) | (4.02) | (4.58) | (111.41) |
| Median | 1.0 | 0.0 | −50.0 | 1.0 | −3.0 | −73.0 | 1.0 | −1.0 | −60.0 |
| Minimum, | 0, 26 | −24, | −100.0, | 0, 30 | −27, | −100.0, | 0, 30 | −27, | −100.0, |
| Maximum | | 21 | 800.0 | | 23 | 850 | | 23 | 850.0 |

Week 40

| | | | | | | | | | |
|---|---|---|---|---|---|---|---|---|---|
| N | 235 | 235 | 170 | 113 | 113 | 102 | 348 | 348 | 272 |
| Mean (SD) | 1.6 | −1.1 | −25.0 | 2.0 | −3.9 | −69.2 | 1.7 | −2.0 | −41.6 |
| | (2.36) | (3.04) | (110.42) | (4.23) | (4.57) | (52.62) | (3.09) | (3.83) | (95.37) |
| Median | 1.0 | 0.0 | −61.3 | 0.0 | −3.0 | −88.9 | 1.0 | −1.0 | −72.1 |
| Minimum, | 0, 14 | −13, | −100.0, | 0, 29 | −23, 6 | −100.0, | 0, 29 | −23, | −100.0, |
| Maximum | | 12 | 600.0 | | | 300.0 | | 12 | 600.0 |

CFBL = change from baseline;
DB = double blinded;
DLQI = Dermatology Life Quality Index;
ITT = Intent-to-Treat;
PGA = Physician Global Assessment;
SD = standard deviation.

Health Related Quality of Life Endpoint, Patient Satisfaction Questionnaire

As of the data cutoff date for this interim analysis, 403 Patient Satisfaction Questionnaires had been completed; the results are presented in Table 18.

The majority of subjects (83.4%) strongly agreed (49.9%) or agreed (33.5%) that they could easily manage their psoriasis with tapinarof cream, 1% and were satisfied (82.4%) with how well tapinarof cream, 1% worked for their psoriasis (strongly agreed: 52.9%; agreed: 29.5%). Subjects randomized to tapinarof cream, 1% in the DB, pivotal Phase 3 studies and subjects randomized to vehicle cream responded similarly.

Of the 353 subjects (88.3%) that had used other topical drugs to treat their psoriasis in the past, 51.5% strongly agreed and 29.3% agreed that tapinarof cream, 1% was more effective than those other topical drugs and 79.2% of subjects preferred tapinarof cream, 1% (strongly agreed: 53.0%; agreed: 26.2%) to the other topical drugs they have used to treat their psoriasis in the past.

Of the 137 subjects (34.2%) that had used systemic drugs to treat their psoriasis in the past, 31.2% strongly agreed and 18.1% agreed that tapinarof cream, 1% was more effective than those systemic drugs and 63.0% of subjects preferred tapinarof cream, 1% (strongly agreed: 37.0%; agreed: 26.1%) to those systemic drugs they have used to treat their psoriasis in the past.

TABLE 18
Summary of Patient Satisfaction Questionnaire at Week 40 or Early
Termination Visit (ITT Population)

| | | | |
|---|---|---|---|
| | Tapinarof | Vehicle | |
| | Cream, 1% | Cream | |
| | (Pivotal) | (Pivotal) | Overall |
| Observed Value, n (%) | (N = 508) | (N = 255) | (N = 763) |

Question 1: I can easily manage my psoriasis with the study drug.

| | | | |
|---|---|---|---|
| Number of Subjects with Known Answer, n | 264 | 139 | 403 |
| Strongly Agree | 129 (48.9) | 72 (51.8) | 201 (49.9) |
| Agree | 93 (35.2) | 42 (30.2) | 135 (33.5) |
| Neutral | 20 (7.6) | 11 (7.9) | 31 (7.7) |
| Disagree | 11 (4.2) | 11 (7.9) | 22 (5.5) |
| Strongly Disagree | 11 (4.2) | 3 (2.2) | 14 (3.5) |

Question 2: The time spent applying the study drug every day was acceptable and did
not affect my everyday life.

| | | | |
|---|---|---|---|
| Number of Subjects with Known Answer, n | 264 | 139 | 403 |
| Strongly Agree | 147 (55.7) | 75 (54.0) | 222 (55.1) |
| Agree | 96 (36.4) | 52 (37.4) | 148 (36.7) |
| Neutral | 12 (4.5) | 11 (7.9) | 23 (5.7) |
| Disagree | 7 (2.7) | 1 (0.7) | 8 (2.0) |
| Strongly Disagree | 2 (0.8) | 0 (0.0) | 2 (0.5) |

Question 3: I am satisfied with how well the study drug worked for my psoriasis.

| | | | |
|---|---|---|---|
| Number of Subjects with Known Answer, n | 264 | 139 | 403 |
| Strongly Agree | 139 (52.7) | 74 (53.2) | 213 (52.9) |
| Agree | 81 (30.7) | 38 (27.3) | 119 (29.5) |
| Neutral | 20 (7.6) | 9 (6.5) | 29 (7.2) |
| Disagree | 16 (6.1) | 14 (10.1) | 30 (7.4) |
| Strongly Disagree | 8 (3.0) | 4 (2.9) | 12 (3.0) |

Question 4: I have confidence in the study drug.

| | | | |
|---|---|---|---|
| Number of Subjects with Known Answer, n | 264 | 139 | 403 |
| Strongly Agree | 141 (53.4) | 72 (51.8) | 213 (52.9) |
| Agree | 78 (29.5) | 40 (28.8) | 118 (29.3) |
| Neutral | 27 (10.2) | 14 (10.1) | 41 (10.2) |
| Disagree | 13 (4.9) | 10 (7.2) | 23 (5.7) |
| Strongly Disagree | 5 (1.9) | 3 (2.2) | 8 (2.0) |

Question 5: The study drug cleared by skin and kept my psoriasis from coming back.

| | | | |
|---|---|---|---|
| Number of Subjects with Known Answer, n | 264 | 139 | 403 |
| Strongly Agree | 73 (27.7) | 42 (30.2) | 115 (28.5) |
| Agree | 90 (34.1) | 42 (30.2) | 132 (32.8) |
| Neutral | 56 (21.2) | 23 (16.5) | 79 (19.6) |
| Disagree | 31 (11.7) | 22 (15.8) | 53 (13.2) |
| Strongly Disagree | 14 (5.3) | 10 (7.2) | 24 (6.0) |

Question 6: If the study drug was available by prescription, I would recommend it to
other patients with psoriasis.

| | | | |
|---|---|---|---|
| Number of Subjects with Known Answer, n | 264 | 139 | 403 |
| Strongly Agree | 139 (52.7) | 72 (51.8) | 211 (52.4) |
| Agree | 81 (30.7) | 39 (28.1) | 120 (29.8) |
| Neutral | 26 (9.8) | 16 (11.5) | 42 (10.4) |
| Disagree | 10 (3.8) | 10 (7.2) | 20 (5.0) |
| Strongly Disagree | 8 (3.0) | 2 (1.4) | 10 (2.5) |

Question 7: If the study drug was available by a prescription, I would use it again or
continue on it.

| | | | |
|---|---|---|---|
| Number of Subjects with Known Answer, n | 264 | 139 | 403 |
| Strongly Agree | 139 (52.7) | 75 (54.0) | 214 (53.1) |
| Agree | 78 (29.5) | 36 (25.9) | 114 (28.3) |
| Neutral | 20 (7.6) | 10 (7.2) | 30 (7.4) |
| Disagree | 18 (6.8) | 12 (8.6) | 30 (7.4) |
| Strongly Disagree | 9 (3.4) | 6 (4.3) | 15 (3.7) |

Question 8: The study drug is easy to apply.

| | | | |
|---|---|---|---|
| Number of Subjects with Known Answer, n | 264 | 139 | 403 |
| Strongly Agree | 158 (59.8) | 80 (57.6) | 238 (59.1) |
| Agree | 97 (36.7) | 52 (37.4) | 149 (37.0) |
| Neutral | 7 (2.7) | 4 (2.9) | 11 (2.7) |
| Disagree | 0 (0.0) | 2 (1.4) | 2 (0.5) |
| Strongly Disagree | 2 (0.8) | 1 (0.7) | 3 (0.7) |

Question 9: The study drug is not greasy.

| | | | |
|---|---|---|---|
| Number of Subjects with Known Answer, n | 264 | 139 | 403 |
| Strongly Agree | 130 (49.2) | 59 (42.4) | 189 (46.9) |
| Agree | 105 (39.8) | 61 (43.9) | 166 (41.2) |
| Neutral | 22 (8.3) | 12 (8.6) | 34 (8.4) |
| Disagree | 6 (2.3) | 7 (5.0) | 13 (3.2) |
| Strongly Disagree | 1 (0.4) | 0 (0.0) | 1 (0.2) |

Question 10: The study drug quickly absorbs into my skin.

| | | | |
|---|---|---|---|
| Number of Subjects with Known Answer, n | 264 | 139 | 403 |
| Strongly Agree | 122 (46.2) | 53 (38.1) | 175 (43.4) |
| Agree | 114 (43.2) | 70 (50.4) | 184 (45.7) |
| Neutral | 19 (7.2) | 12 (8.6) | 31 (7.7) |
| Disagree | 7 (2.7) | 4 (2.9) | 11 (2.7) |
| Strongly Disagree | 2 (0.8) | 0 (0.0) | 2 (0.5) |

Question 11: The study drug feels good on my skin.

| | | | |
|---|---|---|---|
| Number of Subjects with Known Answer, n | 264 | 139 | 403 |
| Strongly Agree | 117 (44.3) | 50 (36.0) | 167 (41.4) |
| Agree | 100 (37.9) | 54 (38.8) | 154 (38.2) |
| Neutral | 39 (14.8) | 28 (20.1) | 67 (16.6) |
| Disagree | 6 (2.3) | 5 (3.6) | 11 (2.7) |
| Strongly Disagree | 2 (0.8) | 2 (1.4) | 4 (1.0) |

Question 12: I am satisfied with the look and feel of the study drug.

| | | | |
|---|---|---|---|
| Number of Subjects with Known Answer, n | 259 | 135 | 394 |
| Strongly Agree | 124 (47.9) | 61 (45.2) | 185 (47.0) |
| Agree | 97 (37.5) | 54 (40.0) | 151 (38.3) |
| Neutral | 34 (13.1) | 15 (11.1) | 49 (12.4) |
| Disagree | 3 (1.2) | 4 (3.0) | 7 (1.8) |
| Strongly Disagree | 1 (0.4) | 1 (0.7) | 2 (0.5) |

Have you used other topical drugs to treat your psoriasis in the past?

| | | | |
|---|---|---|---|
| Number of Subjects with Known Answer, n | 263 | 137 | 400 |
| Yes | 232 (88.2) | 121 (88.3) | 353 (88.3) |
| No | 31 (11.8) | 16 (11.7) | 47 (11.8) |

Question 13: The study drug is more effective than other topical drugs I have used to
treat my psoriasis.

| | | | |
|---|---|---|---|
| Number of Subjects with Known Answer, n | 232 | 123 | 355 |
| Strongly Agree | 117 (50.4) | 66 (53.7) | 183 (51.5) |
| Agree | 68 (29.3) | 36 (29.3) | 104 (29.3) |
| Neutral | 30 (12.9) | 14 (11.4) | 44 (12.4) |
| Disagree | 11 (4.7) | 6 (4.9) | 17 (4.8) |
| Strongly Disagree | 6 (2.6) | 1 (0.8) | 7 (2.0) |

Question 14: The study drug is easier to use than other topical drugs I have used to
treat my psoriasis.

| | | | |
|---|---|---|---|
| Number of Subjects with Known Answer, n | 232 | 123 | 355 |
| Strongly Agree | 72 (31.0) | 46 (37.4) | 118 (33.2) |
| Agree | 76 (32.8) | 38 (30.9) | 114 (32.1) |
| Neutral | 77 (33.2) | 32 (26.0) | 109 (30.7) |
| Disagree | 6 (2.6) | 6 (4.9) | 12 (3.4) |
| Strongly Disagree | 1 (0.4) | 1 (0.8) | 2 (0.6) |

Question 15: I prefer the study drug to other topical drugs I have used to treat my
psoriasis.

| | | | |
|---|---|---|---|
| Number of Subjects with Known Answer, n | 232 | 123 | 355 |
| Strongly Agree | 120 (51.7) | 68 (55.3) | 188 (53.0) |
| Agree | 65 (28.0) | 28 (22.8) | 93 (26.2) |
| Neutral | 32 (13.8) | 16 (13.0) | 48 (13.5) |
| Disagree | 10 (4.3) | 9 (7.3) | 19 (5.4) |
| Strongly Disagree | 5 (2.2) | 2 (1.6) | 7 (2.0) |

Have you used systemic drugs to treat your psoriasis in the past?

| | | | |
|---|---|---|---|
| Number of Subjects with Known Answer, n | 263 | 138 | 401 |
| Yes | 90 (34.2) | 47 (34.1) | 137 (34.2) |
| No | 173 (65.8) | 91 (65.9) | 264 (65.8) |

Question 16: The study drug is more effective than systemic drugs I have used to treat
my psoriasis.

| | | | |
|---|---|---|---|
| Number of Subjects with Known Answer, n | 90 | 48 | 138 |
| Strongly Agree | 33 (36.7) | 10 (20.8) | 43 (31.2) |
| Agree | 16 (17.8) | 9 (18.8) | 25 (18.1) |
| Neutral | 23 (25.6) | 16 (33.3) | 39 (28.3) |
| Disagree | 10 (11.1) | 9 (18.8) | 19 (13.8) |
| Strongly Disagree | 8 (8.9) | 4 (8.3) | 12 (8.7) |

Question 17: The study drug is easier to use than systemic drugs I have used to treat
my psoriasis.

| | | | |
|---|---|---|---|
| Number of Subjects with Known Answer, n | 90 | 48 | 138 |
| Strongly Agree | 29 (32.2) | 15 (31.3) | 44 (31.9) |
| Agree | 28 (31.1) | 14 (29.2) | 42 (30.4) |
| Neutral | 19 (21.1) | 9 (18.8) | 28 (20.3) |
| Disagree | 10 (11.1) | 6 (12.5) | 16 (11.6) |
| Strongly Disagree | 4 (4.4) | 4 (8.3) | 8 (5.8) |

Question 18: I prefer the study drug to systemic drugs I have used to treat my
psoriasis.

| | | | |
|---|---|---|---|
| Number of Subjects with Known Answer, n | 90 | 48 | 138 |
| Strongly Agree | 38 (42.2) | 13 (27.1) | 51 (37.0) |
| Agree | 23 (25.6) | 13 (27.1) | 36 (26.1) |
| Neutral | 14 (15.6) | 10 (20.8) | 24 (17.4) |
| Disagree | 10 (11.1) | 8 (16.7) | 18 (13.0) |
| Strongly Disagree | 5 (5.6) | 4 (8.3) | 9 (6.5) |

cream. Data from both cohorts combined are presented as overall.
DB = double blinded;
ET = early termination;
ITT = Intent-to-Treat;
PGA = Physician Global Assessment.

Discussion

Tapinarof cream, 1% is a nonsteroidal, first in class, therapeutic aryl hydrocarbon receptor modulating agent being developed for the topical treatment of plaque psoriasis in adults. It has the potential to fulfill an unmet medical need as an efficacious topical therapy without restrictions on treatment duration or application site (i.e., use on sensitive areas). This long term extension study confirms the results of the DB, pivotal Phase 3 studies demonstrating that tapinarof cream, 1% has favorable safety, tolerability and efficacy profiles that support long term use for the management of psoriasis.

This report describes a planned interim analysis of this extension study in adults rolling over from the DB, pivotal Phase 3 studies which evaluated tapinarof cream, 1% or vehicle cream in the treatment of plaque psoriasis for 12 weeks. The safety, tolerability, efficacy, durability, and remittive effect of topical tapinarof cream, 1% applied once daily for up to 40 weeks were evaluated in this open label, Phase 3, multicenter long term extension study. Moreover, this extension study was designed to evaluate extended, intermittent use of tapinarof cream, 1% which is more reflective of ‘real world’ application in clinical practice.

Study Population and Baseline Characteristics

Eligibility consisted of those subjects who completed the 12 week treatment period in 1 of the 2 DB, pivotal Phase 3 studies (DMVT 505 3001 or DMVT 505 3002). A total of 763 subjects were enrolled, which included 508 subjects previously randomized to tapinarof cream, 1% and 255 subjects previously randomized to vehicle cream. The population included 235 subjects exposed to tapinarof cream, 1% and followed for 1 year and 450 subjects followed for 6 months which exceeds ICH E1 exposure requirements for safety follow up (ICH, 1994). The full spectrum of PGA scores (clear to severe) was represented at study entry. Subjects previously randomized to vehicle cream in the DB, pivotal Phase 3 studies entered this extension study with a higher disease burden reflective of an untreated population while subjects previously randomized to tapinarof cream, 1% entered with less severe disease reflective of tapinarof cream, 1%'s statistically significant efficacy in the DB, pivotal Phase 3 studies.

The impact of COVID 19 on this study was minimal. No subjects were discontinued due to a positive COVID 19 diagnosis and discontinuations due to other COVID 19 related reasons occurred in <1% of subjects.

Efficacy

Efficacy measures included standard assessments for PGA, % BSA affected, and PASI. Furthermore, the study design allowed for assessment of durability (no loss of effect while on therapy) and remittive effect (maintenance of effect when off therapy).

Approximately 40% of the study population achieved complete clearance (PGA=0) at least once during this extension study; this included 78 subjects that entered the study with a PGA=0 and an additional 221 subjects that entered with a PGA≥1. The high rate of disease clearance with a topical therapy demonstrates a significant opportunity for the care of patients suffering from plaque psoriasis.

Assessment of response over time showed a similar pattern of improvement for PGA, % BSA affected, and PASI. Subjects previously randomized to tapinarof cream, 1% in the DB, pivotal Phase 3 studies demonstrated continued improvement beyond the initial 12 weeks of treatment they received in the DB, pivotal Phase 3 studies before achieving a plateauing of the effect at approximately Week 12 of this extension study (total duration of treatment of 24 weeks). Subjects previously randomized to vehicle cream in the DB, pivotal Phase 3 studies achieved a similar response to those previously randomized to tapinarof cream, 1% in PGA=0 or 1, mean % BSA affected, and mean PASI score by approximately Weeks 24 to 28. Importantly, these beneficial clinical effects did not decline over time, demonstrating that tachyphylaxis was not observed. The durability of effect while on treatment was consistent across all measures of efficacy.

The protocol directed the treatment of subjects entering with a PGA≥1 until they achieved a PGA=0, at which time treatment was suspended. If/when the subject had worsening of disease (PGA≥2), the subject would re initiate treatment until a PGA=0 was achieved. This pattern of treatment, suspension, and re treatment allowed for characterization of a remittive effect, defined as maintenance of clear/almost clear (PGA=0 or 1) while off therapy. In those subjects entering with a PGA=0 (n=78), the median time to worsening of disease was 115 days with a 95% CI ranging from 85 to 162 days. For subjects either entering with or achieving a PGA=0 (n=299), the average treatment free interval was >3 months (mean 97.9 days). The treatment free period is underestimated since some data were censored, meaning that the remittive period ended due to the subject reaching the end of the extension study, not because the subject had worsening disease.

The subject reported DLQI and Patient Satisfaction Questionnaire illustrate the impact of tapinarof cream, 1% in this population. Quality of life measures improved for both DB assigned treatment groups. Initially, greater improvements were observed for subjects previously randomized to vehicle cream in the DB, pivotal Phase 3 studies compared with subjects randomized to tapinarof cream, 1%; however, over time the quality of life improved measurements converged for the 2 DB assigned treatment groups. At Week 40, the overall mean DLQI was 1.7 out of scale that ranges from 0 to 30 with lower scores representing improvement in quality of life measures. The Patient Satisfaction Questionnaire showed a consistently positive perception of tapinarof cream, 1% across all parameters including questions relating to application, ease of use, and disease management.

Final Data Analysis

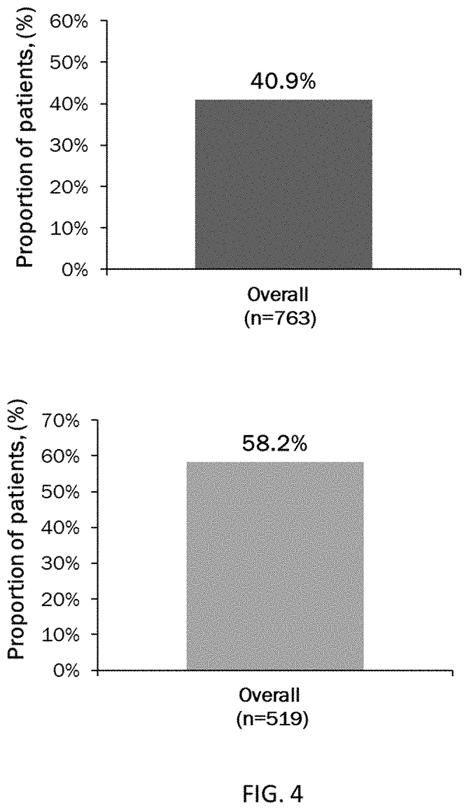

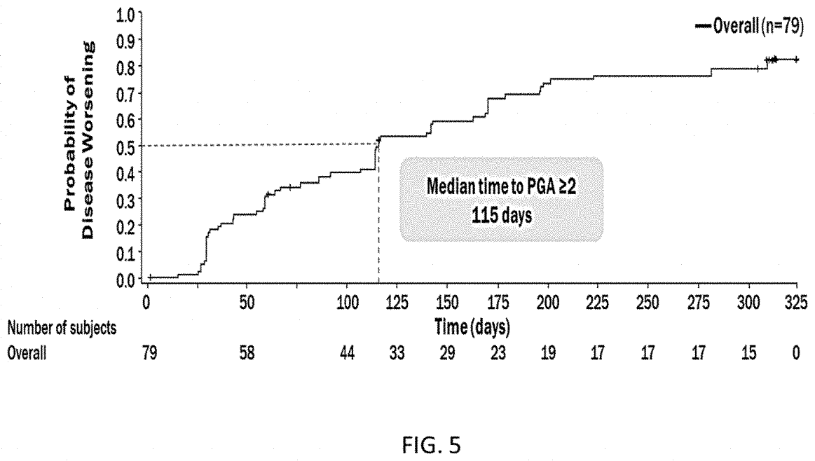

Tapinarof provides for a long-term extension of efficacy measured as complete disease clearance (PGA=0) and response rate. Improvements in efficacy were observed beyond the 12 week pivotal trials. FIG. 4 graphically depicts that 40.9% of patients achieved complete disease clearance at least once during the study, including patients who entered with PGA=0 (n=79) and patients entering with PGA≥1 who achieved PGA=0 at least once during the study (n=233), and that 58.2% of patients entering with PGA≥2 achieved a PGA score of 0 or 1 at least once during the study. As can be seen in FIG. 5, the median time to PGA≥2 off tapinarof was approximately 4 months (115 days) for patients entering with PGA=0 (95% CI: 85 to 168 days) and among patients entering with, or achieving, a PGA=0 (n=312), the mean total duration of remittive effect was approximately 4 months (130 days).

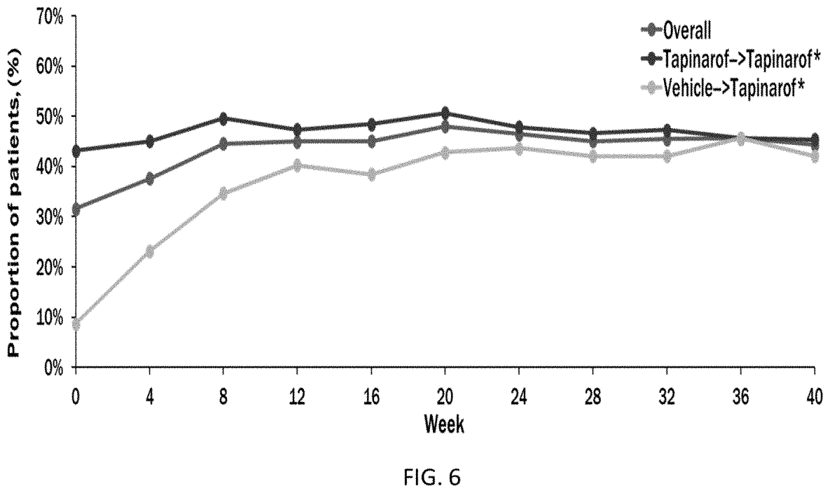

FIG. 6 and Table 19 presents the durability of response of up to 52 weeks, demonstrating no tachyphylaxis over time while on therapy. Patients previously treated with vehicle in the 12-week pivotal trials achieved similar responses to patients previously treated with tapinarof Overall, at baseline, 10.4% of participants had PGA=0 and 31.6% had PGA=0 or 1; at Week 40, 16.9% had PGA=0 and 44.3% had PGA=0 or 1.

TABLE 19
Durability of Response up to 52 weeks

| No. of Subjects | Wk. 0 | Wk. 4 | Wk. 8 | Wk. 12 | Wk. 16 | Wk. 20 | Wk. 24 | Wk. 28 | Wk. 32 | Wk. 36 | Wk. 40 |
|---|---|---|---|---|---|---|---|---|---|---|---|
| Overall | 240 | 273 | 330 | 334 | 336 | 358 | 346 | 336 | 339 | 340 | 330 |
| Tapinarof →Tapinarof | 18 | 216 | 243 | 233 | 239 | 250 | 236 | 230 | 233 | 225 | 224 |
| Vehicle→Tapinarof | 22 | 57 | 87 | 101 | 97 | 108 | 110 | 106 | 106 | 115 | 106 |

Safety

All subjects were included in this interim analysis of safety with exposures in this extension study of up to 40 weeks. Tapinarof cream, 1% was generally safe and well tolerated with long term use. The AE profile was consistent with previous studies and no new safety signals emerged over time. There was a low study discontinuation rate (5.8%) even with extended exposure. All SAEs were reported by the Investigator as not related to study drug. This includes one death from myocardial infarction in a subject with a long standing cardiovascular history who received study drug for 15 weeks.

AESIs of contact dermatitis, folliculitis, and headache were further assessed with long term dosing. Folliculitis remained the most commonly reported TEAE. Importantly, severity did not worsen nor did the incidence increase with long term dosing, and there was a low rate of study discontinuation (1.2%). Similarly, there was no change in the profile of contact dermatitis with regard to severity and incidence and the study discontinuation rate was 1.4%. Headache was a low frequency event with no study discontinuations and negligible impact on treatment.

Tolerability was assessed subjectively via subject and objectively via Investigator throughout the 40 weeks of the study. The patient-reported local tolerability scale utilized for self-reported tolerability assessment is shown in Table 20, while the investigator-assessed local tolerability scale utilized is shown in Table 21.

TABLE 20
Patient-Reported Local Tolerability Scale (Burning/Stinging and
Itching)

| Score | Severity | Description |
|---|---|---|
| 0 | None | Normal, no discomfort |
| 1 | Slight | An awareness, but no discomfort and no intervention |
| | | required |
| 2 | Mild | Noticeable discomfort that causes intermittent |
| | | awareness |
| 3 | Moderate | A noticeable discomfort that causes intermittent |
| | | awareness and interferes occasionally with normal |
| | | daily activities |
| 4 | Strong/ | A definite continuous discomfort that interferes with |
| | Severe | normal daily activities |

TABLE 21
Investigator-Assessed Local Tolerability Scale (Dryness, Erythema and
Peeling)

| Score | Severity | Description |
|---|---|---|
| 0 | No irritation | No evidence of local irritation/intolerance |
| 1 | Mild | Minimal erythema and/or edema, slight glazed |
| | | appearance |
| 2 | Moderate | Definite erythema and/or edema with peeling and/ |
| | | or cracking but does not require treatment |
| | | modification |
| 3 | Severe | Erythema, edema glazing with fissures, few |
| | | vesicles or papules |
| 4 | Very Severe | Strong reaction spreading beyond the treated area, |
| | | bullous reaction, erosions |

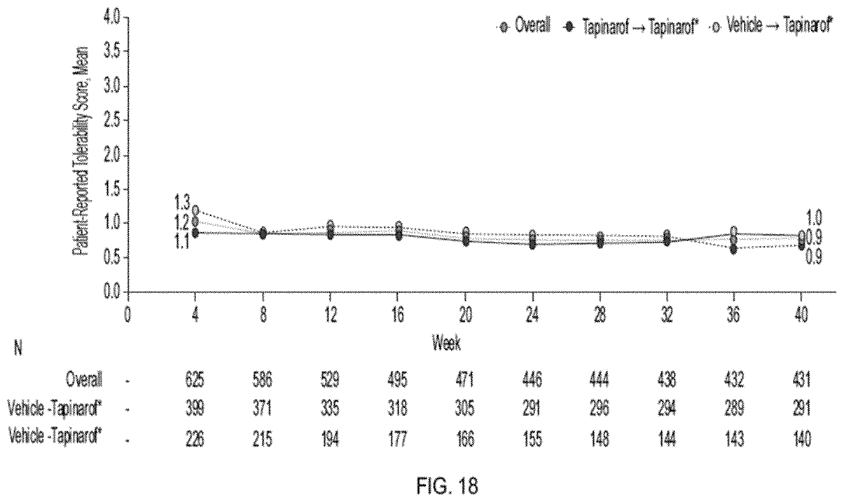

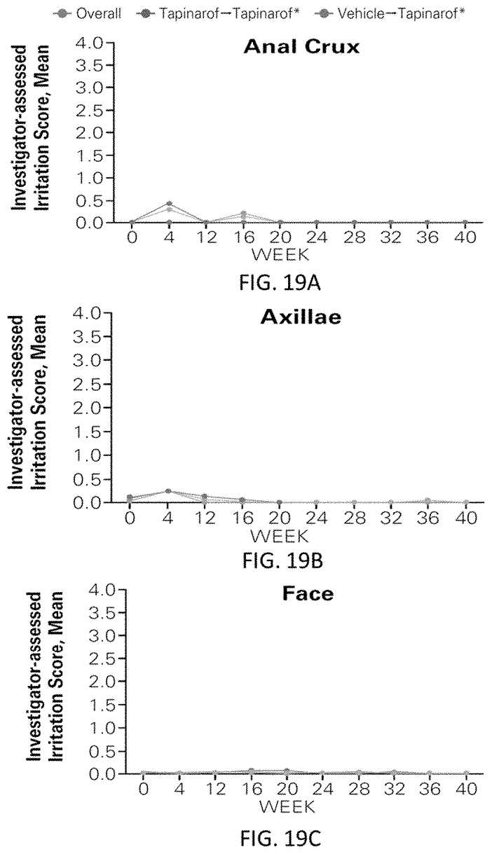

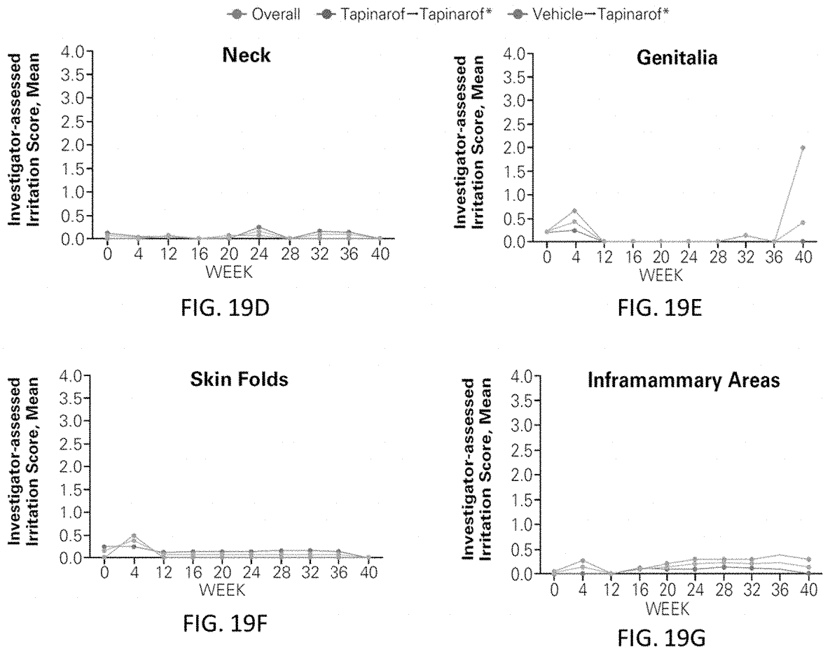

FIG. 18 and FIG. 19 show the tolerability scores reported by the patient and investigator, respectively. These assessments demonstrated a high degree of correlation and both indicated a well-tolerated cream regardless of anatomic region applied, including sensitive areas of the body. There were no clinically relevant trends in laboratory values or vital signs throughout this extension study.

Together, these interim data confirm the favorable safety profile of tapinarof cream, 1% with long term use in adults with plaque psoriasis.

Final analysis provided in Table 22 demonstrates that tapinarof 1% once daily cream was well-tolerated long-term. AEs were consistent with the pivotal trials, with no new safety signals observed with long-term use. TEAEs were mostly mild to moderate, at application sites, and associated with a low discontinuation rate (5.4%). Incidence and severity of folliculitis and contact dermatitis were mild to moderate and associated with low discontinuation rates (1.2% and 1.4%, respectively).

TABLE 22

| | Overall | Tapinarof→Tapinarof | Vehicle→Tapinarof |
|---|---|---|---|
| Patients, n (%) | (n = 763) | (n = 508) | (n = 255) |
| Study | 41 (5.4) | 26 (5.1) | 15 (5.9) |
| discontinuation | | | |
| due to TEAEs | | | |

Treatment-emergent AESI†

| | | | |
|---|---|---|---|
| Folliculitis | 183 (24.0) | 124 (24.4) | 59 (23.1) |
| Dermatitis | 45 (5.9) | 24 (4.7) | 21 (8.2) |
| contact | | | |
| Headache | 15 (2.0) | 10 (2.0) | 5 (2.0) |

Study discontinuation due to AESI

| | | | |
|---|---|---|---|
| Folliculitis | 9 (1.2) | 6 (1.2) | 3 (1.2) |
| Dermatitis | 11 (1.4) | 7 (1.4) | 4 (1.6) |
| contact | | | |
| Headache | 0 | 0 | 0 |

†investigator-specified, may represent multiple MedDRA-preferred terms. ITT population. All AEs reported in the study are considered as TEAE. A patient is counted once only for each MedDRA-preferred term. AE, adverse event; AESI, adverse event of special interest; ITT, intent-to-treat; MedDRA, Medical Dictionary for Regulatory Activities; TEAE, treatment-emergent adverse event.

Overall Conclusions

The interim analysis of this long term extension study demonstrated tapinarof cream, 1% to be generally safe and well tolerated over 40 weeks of once daily application when meeting PGA criteria for treatment. Safety remains consistent over time with a similar AE profile as observed in the DB, pivotal Phase 3 studies and no emergence of new safety signals with long-term use. Tapinarof cream, 1% was well tolerated in all skin locations with extended exposure, including sensitive areas.

Complete clearance of disease (PGA=0) was achieved by 39.2% of subjects (299 of 763 subjects) indicating efficacy continues beyond the 12 week treatment period from the DB, pivotal Phase 3 studies. Efficacy measures did not decline with continued use over time (i.e., no tachyphylaxis). Similar improvements from Baseline were observed in both DB assigned treatment groups with similar effects on PGA, % BSA, and PASI between groups by Week 40.

A treatment-free interval (remittive effect) was observed in subjects that stopped treatment after achieving a PGA=0. In subjects entering the study with a PGA=0 (n=78), the median time to PGA≥2 was approximately 4 months (115 days). Across subjects either entering with or achieving a PGA=0 (n=299), the average treatment free interval was approximately 3 months (mean 97.9 days).

Quality of life measures by DLQI demonstrated improvements from Baseline in both DB assigned treatment groups with similar scores by Week 40.

As mentioned, tapinarof is a novel therapeutic aryl hydrocarbon receptor modulating agent (TAMA) in clinical development for the topical treatment of psoriasis and atopic dermatitis. The efficacy of tapinarof in these inflammatory skin diseases is attributed to tapinarof binding to and activating the ligand-dependent transcription factor aryl hydrocarbon receptor (AhR), resulting in the downregulation of proinflammatory cytokines, normalization of the skin barrier through modulation of skin barrier protein expression, and regulation of gene expression in immune cells. AhR modulates T cell responses both through intrinsic control of T cell subset differentiation and via controlling the function of antigen presenting cells. In addition, AhR signaling promotes the conversion of Th17 cells to type 1 regulatory T cells. Aberrant immune responses underpin the pathogenesis of both PsO and AD, and the wide expression of AhR in immune cells makes it an attractive target given the essential role of AhR in regulating inflammatory responses and skin homeostasis. Tapinarof cream 1% once-daily demonstrated statistically significant efficacy vs vehicle at 12 weeks and was well-tolerated in adults with mild to severe plaque psoriasis in two identical Phase 3 trials, PSOARING 1 and 2. A potential remittive effect was observed in a 12-week phase 2b trial in which efficacy with tapinarof cream 1% was maintained for 4 weeks after treatment cessation. This remittive effect was confirmed in a long-term extension of up to 40 weeks' open-label treatment according to Physician Global Assessment (PGA) score following completion of the 12-week double-blind period in PSOARING 1/2; tapinarof cream 1% demonstrated a high rate of complete disease clearance and a remittive effect of ˜4 months off-therapy, with no tachyphylaxis observed over 52 weeks. These clinical data support the role of AhR in skin immune homeostasis. Given the multitude of AhR-dependent immunoregulatory mechanisms, AhR is an attractive target for novel immunotherapies with the potential to extend therapeutic efficacy following treatment discontinuation; this may provide a unique clinical benefit to patients.

In these two identical, double-blind 12-week pivotal trials (PSOARING 1 and 2), tapinarof cream 1% QD demonstrated significant efficacy versus vehicle and was well tolerated in adults with mild to severe plaque psoriasis. Tapinarof 1% QD showed maintenance of efficacy after treatment discontinuation in a 12-week phase 2b study, warranting investigation of potential remittive effect. Here we present the full results from PSOARING 3, the long-term, open-label, multicenter extension trial assessing the safety, efficacy, tolerability, durability of response, and duration of remittive effect of tapinarof cream 1% QD in adults with mild to moderate plaque psoriasis.

Materials & Methods: Eligible patients completing PSOARING 1 or 2 could enroll in PSOARING 3 for 40 weeks of open-label treatment followed by 4 weeks of follow-up, thus receiving up to 52 weeks of treatment. Patients entering with Physician Global Assessment (PGA) score ≥1 received tapinarof 1% QD until complete disease clearance (PGA=0). Patients entering with, or achieving, PGA=0 discontinued treatment and were monitored for the duration of remittive effect: off-therapy maintenance of PGA=0/1 (clear/almost clear). Patients with disease worsening (PGA≥2) were re-treated with tapinarof until PGA=0. Patients were followed for durability of response on-therapy (no tachyphylaxis). Safety assessments included treatment-emergent adverse events (TEAEs) and patient- and investigator-rated local tolerability. Efficacy endpoints included median time from PGA=0 to first worsening, and proportion of patients with PGA=0/1 after treatment.

Results: 763 patients were enrolled in PSOARING 3, representing 91.6% of eligible patients from the pivotal trials. TEAEs were consistent with the interim analysis and pivotal trials, with no new safety signals observed with long-term use in PSOARING 3. TEAEs were mostly at application sites, mild to moderate in severity, and associated with a low rate of discontinuation (5.8%). Most common TEAEs were folliculitis (22.7%), contact dermatitis (5.5%), and upper respiratory tract infection (4.7%). Incidence and severity of folliculitis and contact dermatitis remained stable with long-term use and were associated with low discontinuation rates (1.2% and 1.4%, respectively). Efficacy measures continued to improve beyond the 12-week pivotal trials: 58.2% of patients entering with PGA≥2 achieved PGA=0/1 at least once during PSOARING 3. Complete disease clearance (PGA=0) was achieved by 40.9% of patients (n=312). For patients entering with PGA=0 (n=79), the median duration of remittive effect was 4 months (115 days). Among patients entering with, or achieving, a PGA=0 (n=312), the mean total duration of remittive effect was approximately 4 months (130 days). Durability of response was demonstrated for up to 52 weeks of treatment, indicating no tachyphylaxis based on the proportion of patients achieving a PGA score of 0 or 1, and maintenance of improvements in % body surface area affected and the Psoriasis Area and Severity Index over time.

Conclusions: Tapinarof cream 1% QD was well tolerated during long-term use, with a safety profile consistent with the previously reported interim analysis and pivotal trials. The confirmed high rate of complete disease clearance, —4-month remittive effect off-therapy, and no tachyphylaxis are key attributes establishing tapinarof as an effective and novel non-steroidal topical psoriasis therapy.

### Example 2: Decline in Tapinarof Plasma Concentrations During a Course of Treatment

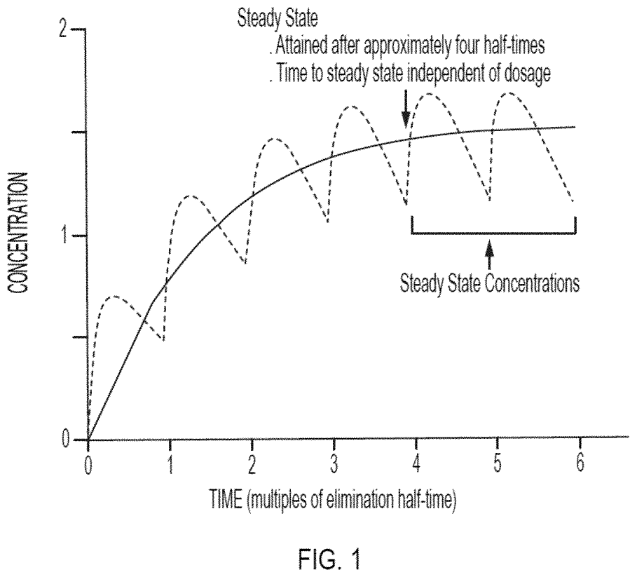

Most drugs follow standard pharmacokinetics where repeated dosing leads to accumulation and eventual achievement of “steady-state.” This term, “steady-state,” implies dosing has continued long enough for the amount of drug administered to equal the amount of drug excreted as shown in FIG. 1.

Unlike most drugs, plasma concentrations of tapinarof have been observed to decrease over the duration of treatment across all clinical studies. This unique PK profile is characterized by the highest plasma concentrations being observed on the first day or week of the treatment period followed by a subsequent decline to undetectable or near undetectable levels in the plasma after multiple weeks of dosing. Accumulation of drug after multiple dosing has not been observed in any clinical study with tapinarof.

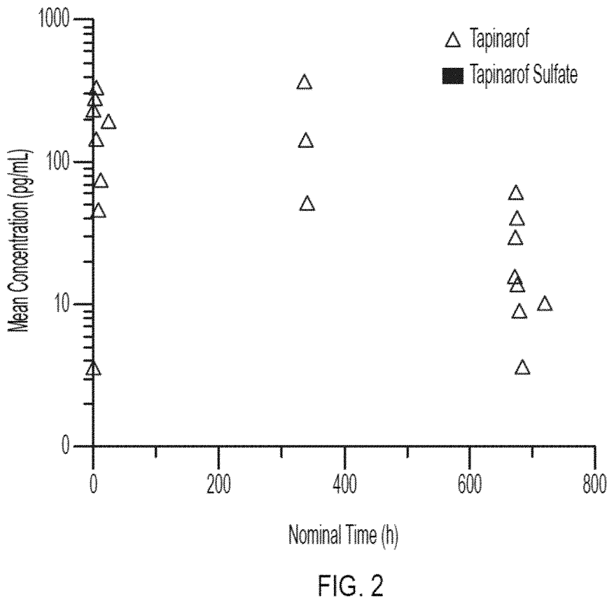

The term steady-state is not applicable to tapinarof because drug concentrations at “steady state” represent a lower level of exposure than drug concentrations seen in the first week of dosing. In the maximal use study with serial PK sampling, a 10-fold decline in concentrations of tapinarof was observed between Days 1 and 29; concentrations of tapinarof sulfate (a metabolite of tapinarof) were not quantifiable. (FIG. 2). This decline is further supported by serial PK data collected from previous clinical studies over 28 days of dosing and over 21 days of dosing. Of note, the reduction in exposure over time appears to occur regardless of formulation, since early studies with a different formulation of tapinarof demonstrated the same effect.

The reason for the decrease in plasma concentrations over time is unknown. Without wishing to be bound by theory, as the skin heals, there may be less penetration of the drug through the stratum corneum and into the systemic circulation since the barrier is restored. Another possibility is induction of CYP1A1/2 enzymes in the skin by tapinarof, which could lead to metabolism of tapinarof in the skin and subsequently decreased levels of tapinarof over the course of treatment; although this mechanism is generally not supported by animal data. Preclinical studies in rats and minipigs show that while CYP1A1/2 are induced in skin or liver, no changes in systemic exposures are observed with repeat dosing compared to single doses. Preclinical studies show that CYP1A1/2 do not play a significant role in metabolic clearance. Furthermore, in vitro studies in human skin microsomes show no metabolic turnover.

The formulation may also play a role. In vitro skin penetration studies show that with improvements in formulation, the amount of drug in dermis increases over time while skin flux decreases. Formulation F with the addition of medium chain triglycerides demonstrated in vitro an increased accumulation into dermis with a decreased amount into the receiving fluid compared to earlier formulations. It should be noted that subjects in clinical studies were instructed to apply study drug to all affected areas including newly appearing lesions and lesions/areas that improved during the study. Therefore, the decreasing concentrations would not be explained by subjects discontinuing study drug once their skin improved. Regardless of the exact mechanism, the plasma exposure of tapinarof has not been shown to be correlated with commonly observed AEs. This PK profile of decreasing concentrations with continued dosing is both uncommon and unexpected. This phenomenon is not generally observed with other drugs, regardless of dosing route. See Duobrii package insert, Vectical Ointment package insert, Bryhali Lotion package insert (all topical treatments for psoriasis). In each of the foregoing, there is accumulation over time with steady-state being reached later in the course of treatment, as would be expected based on standard principles of pharmacokinetics and drug elimination.

### Example 3: Patient Satisfaction with Tapinarof Cream 1% Once Daily for Plaque Psoriasis in a Long-Term Extension Trial

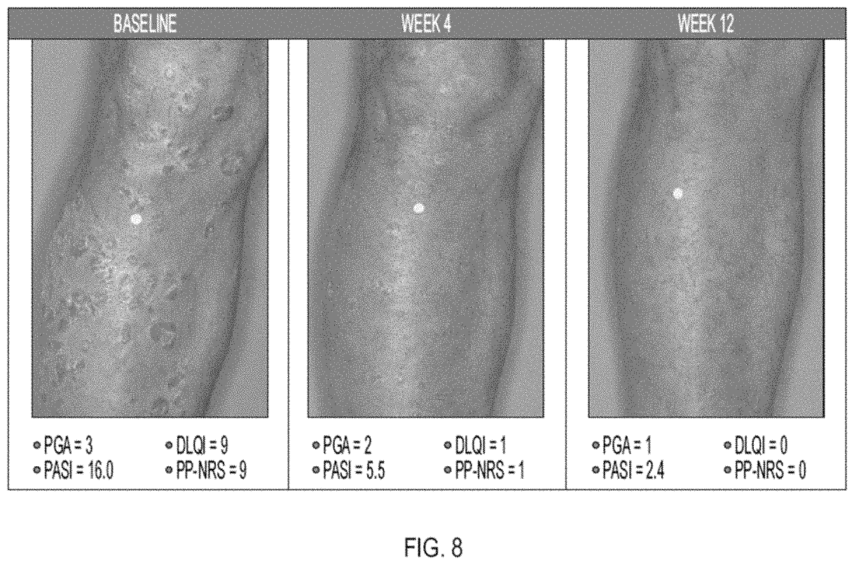

Patient dissatisfaction with current therapies is an important barrier to optimal care of psoriasis (52% of psoriasis patients have reported dissatisfaction with their treatment) and there is a need for efficacious, tolerable, easy to use, topical therapies that can be used long-term, including on sensitive skin areas. Tapinarof 1% is a cosmetically elegant once daily (QD) topical cream that does not contain added fragrance and is free of petrolatum, parabens, and gluten. The vehicle is specifically designed to reduce skin irritation and optimize the delivery of tapinarof to the target site. Tapinarof cream 1% QD demonstrated significant efficacy and was well tolerated, as demonstrated by favorable patient-reported local tolerability and investigator-assessed irritation scores, including on sensitive skin areas, in 1,025 adults with mild-to-severe plaque psoriasis in two 12-week pivotal phase 3 trials, PSOARING 1 (NCT03956355) and PSOARING 2 (NCT03983980). Improvement in psoriasis in a patient achieving the regulatory primary endpoint prior to enrolling in PSOARING 3 is shown in FIG. 8. PSOARING 3 (NCT04053387) is a 40-week long-term open-label extension trial to assess the efficacy, durability of response on therapy, duration of remittive effect off therapy, safety, and tolerability of tapinarof.

Objective: Given that patient dissatisfaction with current therapies has a significant impact on disease management, we assessed patient satisfaction using a Patient Satisfaction Questionnaire; here we present the results from PSOARING 3, a long-term open-label extension trial of tapinarof cream 1% QD.

Patients completing PSOARING 1 and PSOARING 2 were eligible to enroll in PSOARING 3 for up to 40 weeks of open-label treatment with tapinarof cream 1% QD, followed by 4 weeks of follow-up. The Patient Satisfaction Questionnaire was designed to assess patients' satisfaction with the efficacy, formulation elegance, application ease, impact on daily life, and preference for tapinarof versus prior psoriasis therapies. The questionnaire included a series of 18 questions with responses on a scale of strongly agree, agree, neutral, disagree, or strongly disagree. Patient Satisfaction Questionnaire responses were assessed at Week 40 (or Early Termination Visit) and summarized overall.

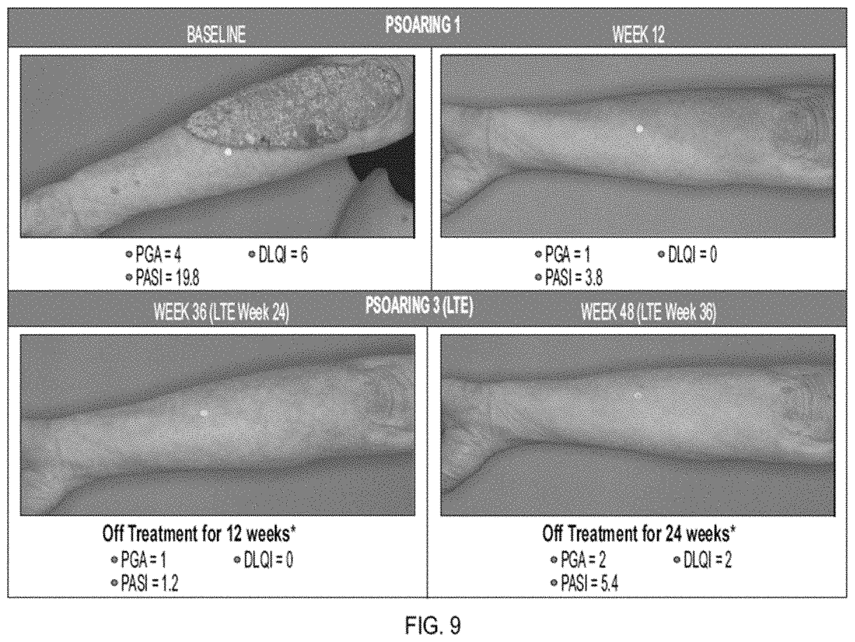

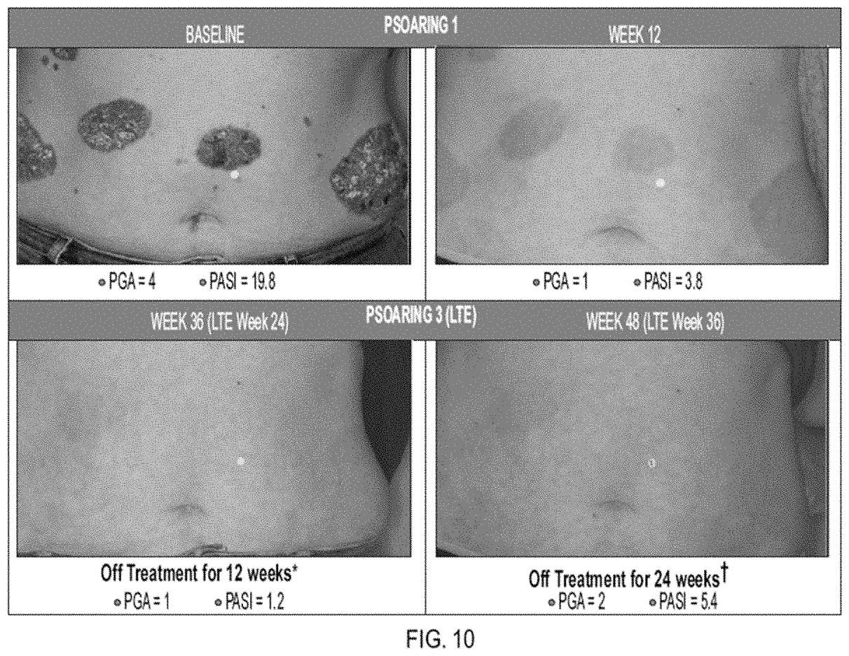

Tapinarof cream 1% QD demonstrated continued and substantial improvement in efficacy with long-term use, beyond the improvements already observed in the 12-week pivotal trials, PSOARING 1 and 2. FIG. 9 and FIG. 10 show improvement in plaque psoriasis in patients who achieved the regulatory primary endpoint after enrollment in PSOARING 3. Both patients achieved the desired remittive effect, being off treatment for 24 weeks prior to commencing with treatment again. Overall, 40.9% of patients (312/763) achieved complete disease clearance at least once during the study; this included 233 patients who entered the study with a PGA of ≥1 and 79 patients who entered with a PGA of 0. The median duration of remittive effect while off therapy for patients who entered the study with a PGA of 0 was 115 days, and the mean total duration of remittive effect for patients who entered with, or achieved, a PGA of 0 was 130 days. Durability of response of up to 52 weeks was demonstrated with intermittent use of tapinarof cream 1% QD, indicating no observation of tachyphylaxis (defined as loss of response) while on therapy. Tapinarof cream 1% QD was well tolerated with long-term use and had a safety profile consistent with previous studies.

Results of Patient Satisfaction Questionnaire: 91.6% of eligible patients (n=763) completing P SOARING 1 and 2 elected to enroll in PSOARING 3. Patient Satisfaction Questionnaires were completed by 78.5% of patients (599/763) in PSOARING 3. Patients consistently reported high satisfaction rates across all parameters, including patients' satisfaction with the efficacy, formulation elegance, application ease, impact on daily life, and preference for tapinarof versus prior psoriasis therapies.

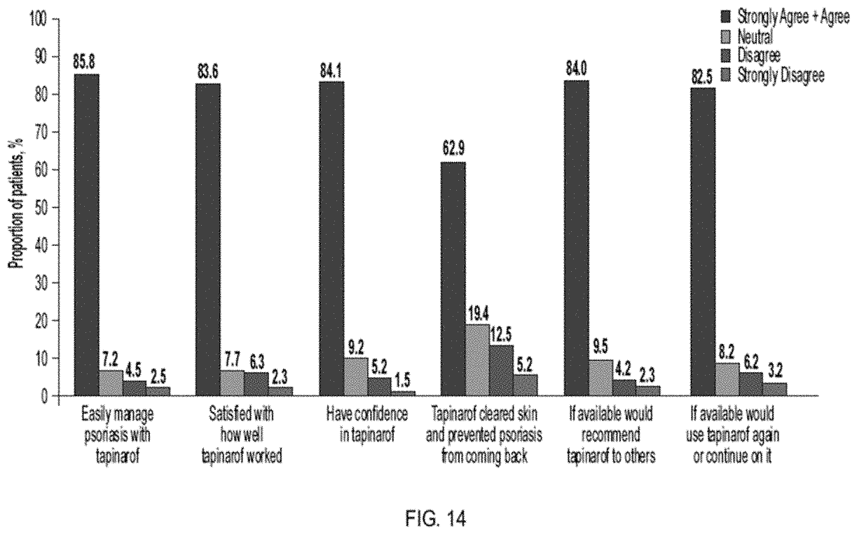

Most patients either strongly agreed or agreed with all questions on confidence and satisfaction with the efficacy of tapinarof (FIG. 14). 85.8% felt they could easily manage their psoriasis with tapinarof, and 83.6% were satisfied with how well tapinarof worked. 62.9% either strongly agreed or agreed that tapinarof cleared their skin and kept psoriasis from coming back, beyond the already observed 40.9% of patients who achieved complete disease clearance and a ˜4-month remittive effect. 84.1% had confidence in tapinarof, and 84.0% would recommend tapinarof to other patients with psoriasis. 82.5% of patients would use tapinarof again or continue on tapinarof if it was available.

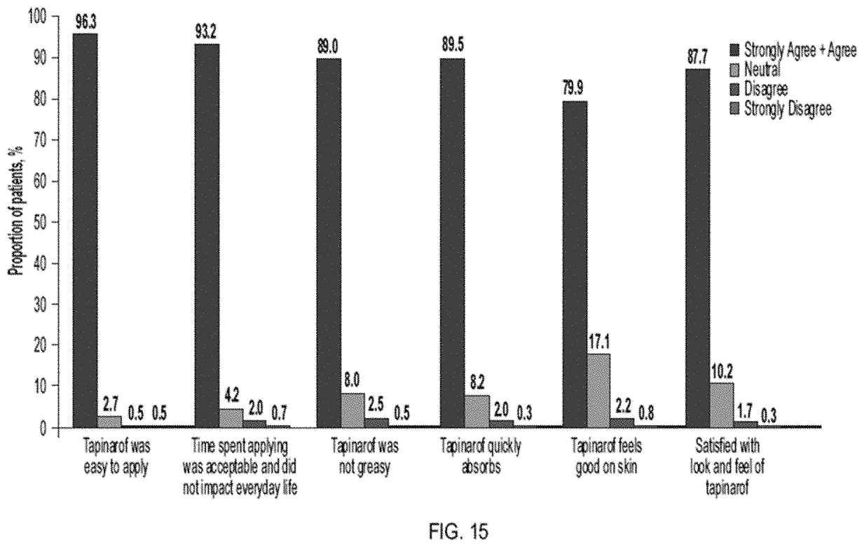

Patients were consistently very satisfied with the tapinarof formulation and elegance (FIG. 15). 93.2% were satisfied with the time spent applying tapinarof cream, and 96.3% considered it easy to apply. In addition, most patients either strongly agreed or agreed that tapinarof was not greasy (89.0%), quickly absorbed (89.5%), and felt good on their skin (79.9%). 87.7% were very satisfied with the look and feel of tapinarof.

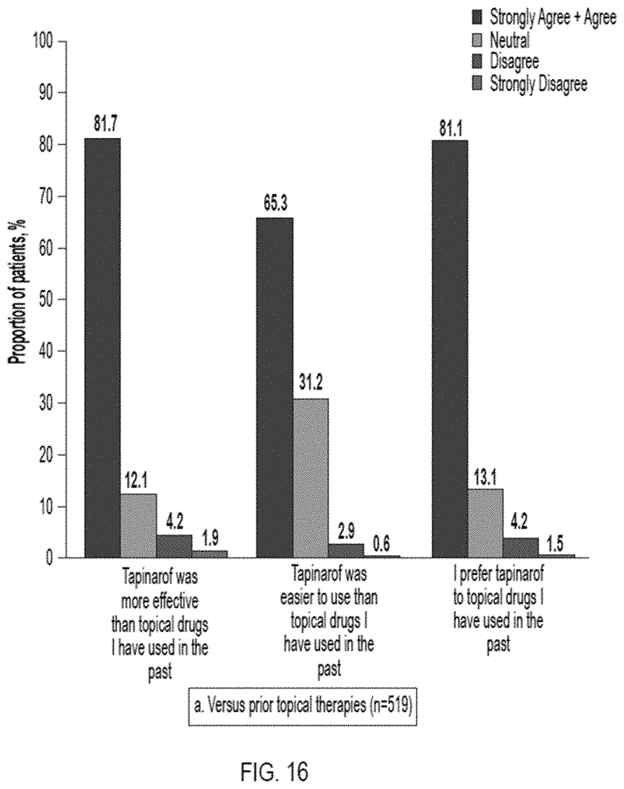

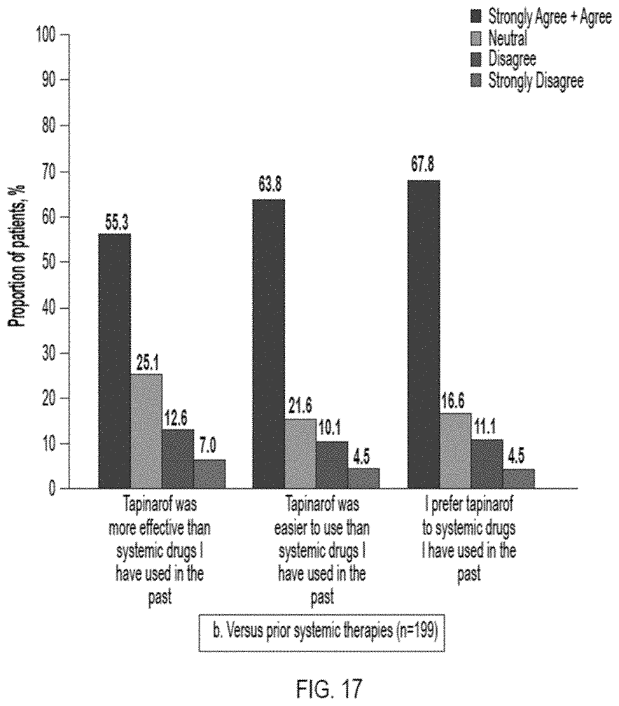

For patients who reported having used other topical drugs to treat psoriasis in the past, 81.7% considered tapinarof to be more effective than prior therapies, and 65.3% considered tapinarof easier to use (FIG. 16). 81.1% of patients preferred tapinarof to other topical drugs used to treat their psoriasis in the past. For patients who reported having used systemic drugs to treat psoriasis in the past, 55.3% considered tapinarof to be more effective than prior therapies, and 63.8% considered tapinarof to be easier to use (FIG. 17). Most patients (67.8%) also preferred tapinarof to systemic drugs used to treat their psoriasis in the past.

Conclusion: Patient satisfaction data from PSOARING 3 demonstrate a consistent and highly positive perception of tapinarof cream 1% QD across all patient relevant parameters, including satisfaction with the efficacy, formulation elegance, application ease, impact on daily life, and preference for tapinarof versus prior psoriasis therapies.

### Example 4: Treat-to-Target Outcomes and Measures of Treatment Success in Three Phase 3 Trials of Tapinarof Cream 1% Once Daily for Mild to Severe Plaque Psoriasis

Introduction

Treat-to-target strategies are used in several chronic diseases to improve outcomes, and treatment goals for psoriasis have been recommended by the US National Psoriasis Foundation (e.g., achieving a percentage body surface area (% BSA) affected of ≤1% at 3 months) and the European S3-Guidelines on the Systemic Treatment of Psoriasis Vulgaris supported by the EDF/EADV/IPC (e.g., a ≥75% decrease in Psoriasis Area and Severity Index [PASI75] within 3-4 months). In clinical practice, these targets can be difficult for many patients to achieve, and current topical treatments alone are generally insufficiently efficacious to achieve these goals. Tapinarof cream 1% once daily (QD) demonstrated statistically significant efficacy versus vehicle and was well-tolerated in adults with mild to severe plaque psoriasis in two identically designed, 12-week, phase 3 trials, referred to as PSOARING 1 & 2. Eligible patients who completed PSOARING 1 & 2 could enroll in PSOARING 3, a long-term extension trial of up to 40 weeks of intermittent treatment with tapinarof cream 1% QD based on patients' Physician Global Assessment (PGA) scores. In PSOARING 1 & 2, both primary (PGA=0 or 1 and a ≥2-grade improvement from baseline) and secondary endpoints (PASI75) were met at Week 12, with statistical significance in favor of tapinarof. Efficacy continued to improve in PSOARING 3 beyond the 12-week trials, with a high rate of complete disease clearance (PGA=0), ˜4-month remittive effect off therapy, and durability on therapy, even with long-term use.

Materials and Methods

Treat-to-target outcomes for patients treated with tapinarof cream 1% QD in the PSOARING trials were analyzed. In addition to a % BSA of ≤1.0% and PASI75, pooled analyses explored more aggressive targets, including the proportion of patients with % BSA of ≤0.5%, or an absolute PASI score of ≤1, ≤2 or ≤3. Time-to-event analyses are based on Kaplan-Meier (KM) estimates using observed cases among all patients in the PSOARING trials who had a baseline PGA≥2 before tapinarof treatment. Safety analyses are based on all patients who received tapinarof in the PSOARING trials.

Results

Efficacy analyses included 915 patients. Results indicated that 49.7% of patients (n=455) achieved % BSA≤0.5%, with a KM estimated median time to target of 199 days (95% confidence interval, 172-228); 61.3% (n=561) achieved % BSA≤1.0%, median time to target of 120 days (113-141). In addition, 50.3% of patients (n=460) achieved PASI≤1, median time to target of 185 days (169-218); 66.9% (n=612) achieved PASI≤2, median time to target of 87 days (85-110); and 75.0% (n=686) achieved PASI≤3, mean time to target of 58 days (57-63). At baseline, 86.1% of patients were aged ≤65 years, 57.6% had disease duration >10 years, 78.1% had PGA=3 (moderate), overall mean PASI was 8.7, and overall mean % BSA was 7.8%. Among all patients who received tapinarof cream 1% QD in the PSOARING trials (N=936), most treatment-emergent adverse events (TEAEs) were mild to moderate and rarely led to trial discontinuation. Most common TEAEs (in ≥5% of patients) were folliculitis, contact dermatitis, and nasopharyngitis.

Discussion

Intermittent treatment with topical tapinarof cream 1% QD alone achieved multiple treatment targets in a high percentage of patients. Together with previously reported efficacy and safety, these findings demonstrate that patients treated with tapinarof monotherapy can achieve and exceed ambitious treatment targets. The aggressive target of ≤1% BSA affected was achieved by 40% of tapinarof-treated patients within ˜3 months; attainment continued to increase over time, with 61% of patients achieving this target at ˜4 months. These findings support continuing tapinarof treatment beyond 3 months for patients who are experiencing improvement but not yet meeting the ≤1% BSA affected treatment target advocated by the National Psoriasis Foundation. n absolute PASI total score of ≤2 was achieved by 67% of tapinarof-treated patients; this is a treatment target that has been shown to correspond to a PASI90 response. These analyses may have underestimated the percentage of patients who achieved treatment targets, due to the unique forced-withdrawal design of PSOARING 3 that resulted in intermittent tapinarof treatment (treatment withdrawal when patients achieved PGA=0).

### Example 5: Tapinarof Cream 1% Once Daily for Plaque Psoriasis: Improvements in Quality of Life and Clinical Efficacy in Two Pivotal Phase 3 Trials

Introduction

Tapinarof cream 1% once daily (QD), a topical aryl hydrocarbon receptor (AhR)-modulating agent, demonstrated highly statistically significant efficacy versus vehicle and was well-tolerated in adults with mild to severe plaque psoriasis in two identically designed, 12-week, pivotal phase 3 trials, referred to as PSOARING 1 & 2. The primary efficacy endpoint of Physician Global Assessment (PGA) response, defined as a PGA score of 0 (clear) or 1 (almost clear) and ≥2-grade improvement from baseline at Week 12, was achieved by 35.4% and 40.2% of patients in the tapinarof groups versus 6.0% and 6.3% in the vehicle groups (P<0.0001 for both comparisons) in each trial, respectively. All secondary endpoints were met, with statistical significance. In addition to superiority on clinical efficacy endpoints, significantly greater improvements in patient-reported Dermatology Life Quality Index (DLQI) change from baseline at Week 12 were observed with tapinarof versus vehicle. Here, we present further analyses of the DLQI results, including an evaluation of correlations between the DLQI and clinical efficacy as assessed by PGA and Psoriasis Area and Severity Index (PASI) scores in PSOARING 1 & 2.

Materials and Methods

Patients in PSOARING 1 & 2 were randomized to tapinarof cream 1% QD or vehicle for 12 weeks. The DLQI is a 10-item scale where each item rates impact on quality of life (QoL) on a 4-point scale from 0 (not at all) to 3 (very much); lower scores indicate higher QoL. Efficacy was evaluated using PGA and PASI. Spearman rank correlations were used to evaluate correlations between changes from baseline in efficacy and changes from baseline in QoL at Week 12. Analyses used observed cases and were based on the intention-to-treat population.

Results

683 tapinarof (340 PSOARING 1; 343 PSOARING 2) and 342 vehicle-treated patients (170; 172) were included in the analyses. At baseline in PSOARING 1 & 2, 79.2% and 83.9% of patients had a PGA of 3 (moderate) and mean PASI of 8.9 and 9.1, respectively; mean DLQI was 8.2-8.7 across treatment groups and trials. The mean change in DLQI from baseline at Week 12 was ˜5.0 vs ˜3.0 (P<0.0001) and ˜4.7 vs ˜1.6 (P<0.0001), with tapinarof versus vehicle in each trial, respectively. The minimal clinically important difference in DLQI of 4 was exceeded at Week 12 in the tapinarof groups. A significantly higher proportion of patients achieved a DLQI of 0 or 1 at Week 12 in the tapinarof groups versus vehicle: 47.4% vs 23.3% (P<0.0001) and 44.9% vs 16.1% (P<0.0001) in each trial, respectively; statistical significance in favor of tapinarof was observed as early as Week 4. Improvements in the DLQI in the tapinarof groups at Week 12 were statistically correlated with improvements in PGA (0.28 and 0.29, P<0.0001) and PASI (0.28 and 0.40, P<0.0001) in each trial, respectively.

Discussion

Tapinarof cream 1% QD demonstrated rapid, clinically meaningful, and statistically significant improvements in investigator-assessed objective efficacy measures and patient-reported QoL. A large percentage (45%-47%) of tapinarof-treated patients achieved a DLQI of 0 or 1, i.e., no negative effects on patients' QoL. The statistical correlations between improvements in DLQI and investigator-assessed efficacy measures suggest that, beyond clinical improvements captured by the PASI and PGA, other important factors such as mental/emotional well-being and satisfaction with treatment may contribute to the considerable overall improvement in QoL observed in these trials.

### Example 6: A Long-Term, Open-Label, Extension Study to Evaluate the Safety and Efficacy of Tapinarof Cream, 1% for the Treatment of Atopic Dermatitis in Adults

Background

Atopic dermatitis (AD) (also called atopic eczema) is an intensely pruritic, chronic, relapsing, inflammatory skin disease. The characteristic signs and symptoms of AD include sensations of pruritis and burning, xerosis, erythematous papules and plaques, exudation, crusting, and lichenification. Quality of life is affected through sleep deprivation due to the intense and constant itching, as well as the stigma associated with having a visible skin disease. Up to 30% of children may be affected by AD at some point, and 2% to 10% of adults have AD. Currently there is no curative therapy. Stabilizing the disease and reducing the number and severity of flares are the primary goals of treatment. Topical treatments directed at skin inflammation are a key factor in disease management, as is symptomatic relief of itching. Although multiple topical treatment options are available, there still remains a need for a topical treatment that combines a high level of efficacy with an acceptable safety profile that permits application to a large body surface area (BSA) without restrictions on duration of treatment.

Tapinarof cream, 1% is a white to off-white, oil-in-water emulsion intended for topical application to AD and psoriatic skin lesions, which has a novel mechanism of action. Tapinarof is a nonsteroidal, small molecule therapeutic aryl hydrocarbon receptor (AhR) modulating agent (TAMA), which likely exerts its therapeutic effects via agonism of AhR, a cytosolic ligand dependent transcription factor. Upon ligand binding, AhR translocates to the nucleus and dimerizes with AhR nuclear translocator (ARNT) forming an AhR ARNT heterodimer. The AhR ARNT heterodimer modulates gene transcription through direct and indirect interaction with DNA. By activation of this AhR signaling pathway, tapinarof has the potential to downregulate the expression of pro inflammatory cytokines, including interleukin (IL)-4, IL-5, IL-6, IL-13, IL 17A and F, and eotaxin, and upregulate the expression of several skin barrier proteins, including filaggrin, hornerin, and involucrin. In addition, AhR also activates the antioxidative transcription factor nuclear factor-erythroid 2-related factor-2 (Nrf2), upregulating the expression of antioxidative enzymes. By targeting AhR, tapinarof has a biological profile that differs from that of currently available products, offering patients a truly novel therapeutic treatment option for plaque psoriasis and AD.

Two Phase 2b, 12-week, randomized, double-blind, vehicle-controlled, 6 arm, parallel group, dose finding studies with topically applied tapinarof cream were conducted by GlaxoSmithKline (GSK); 1 study each in subjects with AD or psoriasis. These 2 studies evaluated the safety and efficacy of tapinarof cream (Formulation F) at 2 concentrations (0.5% or 1% weight/weight [w/w]) and 2 application frequencies (once daily [QD] or twice daily [BID]) in 247 adult and adolescent subjects with AD and in 227 adult subjects with plaque psoriasis. In both studies, tapinarof showed a clear therapeutic effect compared with vehicle, with the 1% w/w concentration treatment groups demonstrating a higher proportion of subjects with treatment success compared with the 0.5% w/w concentration groups (applied QD and BID in the AD study). In both indications, the tapinarof 1% dosing groups showed a faster onset of action than the 0.5% dosing groups, and QD application had similar efficacy to BID application. In both Phase 2b studies, tapinarof showed an acceptable safety profile. Treatment-emergent adverse events (TEAEs) were reported with a higher frequency in the tapinarof groups than in the vehicle groups. The most frequent TEAEs (≥5% in any arm or in total) were nasopharyngitis, folliculitis, dermatitis contact, atopic dermatitis, upper respiratory tract infection, headache, vomiting, acne, application site dermatitis, miliaria, dermatitis allergic, and impetigo. The majority of TEAEs were mild or moderate in severity. In each study, the tapinarof 1% QD treatment group had a lower frequency of TEAEs than the tapinarof 1% BID treatment group.

Tapinarof has been evaluated in two identical Phase 3, 12-week, randomized, double-blind, vehicle-controlled trials in adult subjects with plaque psoriasis. These studies evaluated the safety and efficacy of tapinarof cream, 1% QD versus vehicle cream QD in a combined 1025 subjects randomized 2:1 tapinarof to vehicle. In both trials, tapinarof demonstrated superiority against vehicle on the primary endpoint, a Physician Global Assessment (PGA) score of 0 (clear) or 1 (almost clear) with a 2-point improvement at Week 12. In addition, tapinarof was highly statistically significant compared to vehicle on all secondary endpoints which included proportion of subjects achieving a 75% improvement in the Psoriasis Area and Severity Index, a PGA score of 0 or 1, change in percent body surface area (% BSA), and proportion of subjects achieving a 90% improvement in the Psoriasis Area and Severity Index at Week 12. TEAEs were reported with a higher frequency in the tapinarof group than in the vehicle group. The most frequent TEAEs (≥5% in any arm or in total) were folliculitis, nasopharyngitis, and contact dermatitis. The majority of TEAEs were mild or moderate in intensity.

Tapinarof has also been evaluated in an open-label, long-term extension (OL-LTE) study in adult subjects with plaque psoriasis. The OL-LTE study in adults with plaque psoriasis evaluated the safety and efficacy of tapinarof cream, 1% QD for 40 weeks in 763 subjects enrolled from the two identical 12-week studies. While the study remains ongoing at this time, in a pre-specified interim analysis of the study tapinarof demonstrated continued and substantial improvement in efficacy endpoints beyond the 12 weeks of treatment observed in the identical Phase 3 studies. A high rate of complete disease clearance was achieved, with a remittive effect of approximately 4 months of disease control off therapy. Tapinarof was well tolerated with long-term use and had a safety profile consistent with previous studies.

Study Rationale

Twin pivotal Phase 3 studies (Phase 3 pivotal safety and efficacy study DMVT-505-3101, Phase 3 pivotal safety and efficacy study DMVT-505-3102) are being conducted as part of a clinical development program to evaluate the efficacy and safety of tapinarof cream, 1% for the topical treatment of AD in adults and children ages 2 years and above. The results of this study are intended to support product registration in the United States and Canada.

A 48-week, Phase 3, OL-LTE study will also be conducted as part of a clinical development program to evaluate the long-term safety and continued efficacy of tapinarof cream, 1% for the treatment of AD in children and adults. Subjects who complete one of three parent studies (Phase 3 pivotal safety and efficacy study DMVT-505-3101, Phase 3 pivotal safety and efficacy study DMVT-505-3102, or Phase 2 maximal use pharmacokinetics [PK] study DMVT-505-2104) and meet the predefined criteria will have the option to participate in this study. Additionally, approximately 125 pediatric subjects ages 2 to <18 years who are not eligible for participation in the Phase 3 pivotal studies will also be enrolled directly into this OL-LTE study.

Rationale for Study Design, Dose, and Control Groups

The Phase 3 pivotal studies (Phase 3 pivotal safety and efficacy study DMVT-505-3101, Phase 3 pivotal safety and efficacy study DMVT-505-3102, both for the topical treatment of AD) are both 8-week double-blind, vehicle-controlled treatment studies in which subjects will be randomized to receive tapinarof cream, 1% or vehicle cream QD for 8 weeks. The studies will be conducted at multiple study sites in more than one country to enhance the possibility of inclusion of a wider range of population groups and to increase generalizability of the results. This randomized, double-blind, vehicle-controlled study design will minimize the potential for subjective bias related to possible identification of which subjects are receiving active treatment and will minimize selection and allocation bias by balancing potential prognostic factors. A vehicle control group is included to provide a control for comparison and to ensure study sensitivity for characterization of the safety and efficacy profile of tapinarof cream, 1%.

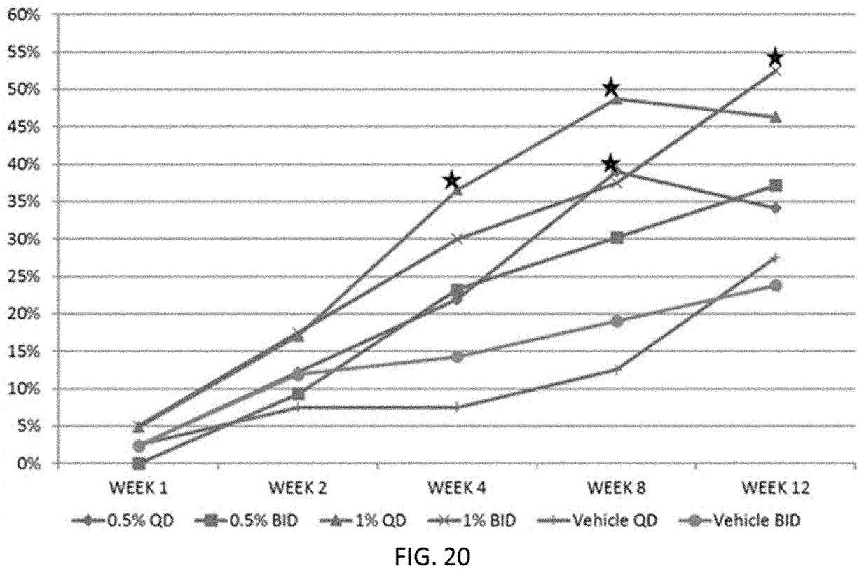

Tapinarof cream has been investigated for the topical treatment of AD in prior studies at concentrations of up to 8.0% w/w in nonclinical studies and 2.0% w/w in clinical studies at QD and BID dosing frequencies. The 1% concentration applied QD was chosen as the dose and dosing frequency to take forward in the Phase 3 clinical development program based on the data from GSK Phase 2b clinical Study 203121. In that study, applications of tapinarof cream at concentrations of 0.5% and 1% applied QD or BID were evaluated. Treatment success as defined by an Investigator's Global Assessment (IGA) score of clear or almost clear with a minimum 2-point improvement was higher with both tapinarof concentrations than with vehicle at all visits beyond Week 2, as shown in FIG. 20, which shows treatment success (IGA score) by dosing regimen in Study 203121. The groups treated with a 1% concentration showed higher efficacy and resulted in a quicker onset of effect than did the groups treated with a 0.5% concentration. Efficacy responses began as early as Week 2 and continued through Week 12. The proportion subjects with ≥75% improvement in Eczema Area Severity Index (EASI) was also higher for the 1% BID (60%) and 1% QD (51%) compared to vehicle (25-26%). Overall, both concentrations demonstrated an acceptable safety profile when applied once or BID with the 1% QD arm having a lower number of reported TEAEs compared to the 1% BID arm (54% vs 70%, respectively). QD application was selected as efficacy was similar between 1% BID and 1% QD regimens and provided a better safety profile. In addition, QD application may improve treatment adherence compared with more frequent dosing administrations.

The 8-week treatment endpoint for the Phase 3 pivotal studies was selected based on similar response rates between week 8 and week 12 for IGA and EASI endpoints. Treatment success for IGA was 49% at Week 8 and 46% at Week 12 for the 1% QD arm. Similarly, the proportion subjects with ≥75% improvement in EASI was 57% at both Week 8 and Week 12. The effect of vehicle continued to increase between these timepoints. Clinical studies in AD have historically shown a notable vehicle response rate that could be attributable to the effects of skin moisturization or to the increased emphasis on proper skin care while participating in a clinical study. An 8-week duration may best show differences between active and vehicle dosing groups, demonstrating the true drug effect.

Based on the efficacy and safety data from Study 203121, tapinarof cream, 1% QD was chosen as the concentration and dosing frequency to be investigated in the Phase 3 pivotal studies and intended for registration. The dose selected from adolescent and adult data is likely to be effective and safe in younger pediatric populations based on similarities in skin anatomical and functional maturity, and our current understanding of the mechanism of action. Furthermore, extrapolation of data from the Phase 3 program into this younger population is supported by similarities in disease definition, clinical classification, measures of disease progression, and pathophysiology.

In GSK study 203121, the safety, efficacy, and plasma concentrations of tapinarof were similar between adults and adolescents. Adolescent subjects had a similar IGA score and % BSA affected at baseline relative to adults. The incidence and type of adverse events (AEs) in the adult and adolescent populations were consistent with that seen in the overall population. In addition, plasma concentrations were similar between adults and adolescents, with both groups having minimal or no absorption.

Evaluation of the clinical pharmacology of tapinarof across 10 studies demonstrate the skin as the site of drug action, minimal systemic absorption following topical dosing, and no accumulation with repeat QD dosing. The skin of infants would be expected to have similar barrier function to adults. Infants born at 30 and 32 weeks gestational age were found to have a fully functional stratum corneum comparable with that of adults. Another study demonstrated that the way the stratum corneum stores and transports water becomes adult-like after the first year of life.

Given the low systemic absorption of tapinarof observed in adults and adolescents and the fact the skin barrier function in the age group being evaluated is expected to be similar to that of adults, absorption of tapinarof in pediatric patients is expected to be low and not impacted by BSA:bodyweight ratios. The sponsor has evaluated various relationships between pharmacokinetics (when detectable) and patient factors. An analysis from the psoriasis maximal use study demonstrated no relationship between subjects' % BSA affected and tapinarof exposure. Furthermore, no clinically relevant differences in plasma concentration were observed based on age, sex, race, and % BSA affected in the Phase 3 pivotal trials in patients with psoriasis.

Potential relationships between tapinarof plasma exposure and measures of efficacy and safety in subjects with psoriasis and AD were explored in an exposure response analysis across Phase 2 and 3 studies. No significant correlations between plasma exposure and adverse events of special interest (AESIs) or efficacy were observed. Thus, the minimal systemic exposure and lack of any pharmacokinetic/pharmacodynamic relationships further support usage of tapinarof down to 2 years of age.

As in the three parent studies, the Phase 3 OL-LTE study will be conducted at multiple study sites in more than one country to enhance the possibility of inclusion of a wider range of population groups and to subsequently increase generalizability of the results. Tapinarof cream is intended for long-term, intermittent use in non-life-threatening inflammatory dermatologic conditions. Therefore, the 48-week duration of long-term, intermittent use of tapinarof cream, 1% in this study is expected to be an adequate duration to assess safety and efficacy of repeated treatment courses of tapinarof as recommended in the International Conference on Harmonisation E1A (ICH E1A) guideline on the extent of population exposure to assess clinical safety for drugs intended for long-term treatment of non-life-threatening conditions, which recommends a minimum of 100 subjects treated with the intended clinical dose to be followed for at least one year.

Phase 3 pivotal study objective: To evaluate the efficacy of tapinarof cream, 1% QD compared with vehicle control in subjects with AD. Endpoint: Proportion of subjects who have a validated Investigator Global Assessment in Atopic Dermatitis (vIGA-AD) score of clear or almost clear (0 or 1) and at least a 2-grade reduction from Baseline at Week 8.

Phase 3 pivotal study objective: To further characterize the efficacy of tapinarof cream, 1% QD compared with vehicle control over time. Endpoints: Proportion of subjects with ≥75% improvement in EASI from Baseline at Week 8; Mean change in % BSA affected from Baseline at Week 8; Proportion of subjects with ≥90% improvement in EASI from Baseline at Week 8; Proportion of subjects ≥12 years old with a Baseline Peak Pruritus-Numeric Rating Scale (PP-NRS) score ≥4 who achieve ≥4-point reduction in the PP-NRS from Baseline at Week 8; Time to achieve a vIGA-AD score of 0 or 1 and at least a 2-grade reduction from Baseline; Proportion of subjects who achieve a vIGA-AD score of clear or almost clear (0 or 1) and at least a 2-grade reduction from Baseline at Weeks 1, 2, and 4; Proportion of subjects with ≥50% improvement in EASI from Baseline at each study visit; Proportion of subjects with ≥75% improvement in EASI from Baseline at Weeks 1, 2, and 4; Proportion of subjects with ≥90% improvement in EASI from Baseline at Weeks 1, 2, and 4; Mean change in % BSA affected from Baseline at Weeks 1, 2, and 4; Mean percent change in % BSA affected from Baseline at each study visit; Mean change and percent change in BSA×vIGA-AD from Baseline at each study visit; Mean change and percent change in EASI score from Baseline at each study visit; vIGA-AD scores and change from Baseline at each study visit; Mean change in PP-NRS score from Baseline at each study visit; Proportion of subjects with a Baseline PP-NRS score ≥4 who achieve ≥4-point reduction in the PP-NRS from Baseline at each study visit; Proportion of subjects ≥12 years old with a Baseline PP-NRS score ≥4 who achieve ≥4-point reduction in the PP-NRS from Baseline at Weeks, 1, 2, and 4; Proportion of subjects <12 years old with a Baseline PP-NRS score ≥4 who achieve ≥4-point reduction in the PP-NRS from Baseline at each study visit.

Phase 3 pivotal study objective: To evaluate the safety and tolerability of tapinarof cream, 1% QD in subjects with AD. Endpoints: Incidence, frequency, and duration of TEAEs and serious adverse events (SAEs); Change from Baseline in laboratory values; Change from Baseline in ECG parameters; Change from Baseline in vital signs; Mean Investigator-assessed local tolerability scale (LTS) scores by visit (overall and sensitive areas); Mean subject (or caregiver)-assessed LTS scores by visit.

Phase 3 pivotal study objective: To describe the effect of tapinarof cream, 1% QD on AD symptom severity and the associated impact on daily activities and attitudes in subjects with AD. Endpoints: Mean change from Baseline in Dermatology Life Quality Index (DLQI), Children's Dermatology Life Quality Index (CDLQI), and Infant's Dermatitis Quality of Life Index (IDQOL) total and individual domain scores at each study visit; Mean change from Baseline in EQ-5D-5L and EQ-5D-Y total and individual domain scores at each study visit; Mean change from Baseline in the total score of the Patient Oriented Eczema Measure (POEM) For Self-Completion (subjects ≥12 years old) at each study visit; Mean change from Baseline in the total score of the POEM For Proxy-Completion (by caregiver for subjects <12 years old) at each study visit; Mean change from Baseline in the total score of the Dermatitis Family Impact (DFI) at each study visit.

Phase 3 pivotal study objective: To evaluate plasma concentrations of tapinarof in subjects with AD. Endpoint: Plasma concentration of tapinarof at Weeks 4 and 8.

Phase 3 OL-LTE study objective: To evaluate the safety and tolerability of tapinarof cream, 1% in subjects with AD. Endpoints: Incidence, frequency, and duration of TEAEs and serious adverse events (SAEs); Change from Baseline in laboratory values; Change from Baseline in vital signs; Mean Investigator-assessed local tolerability scale (LTS) scores by visit (overall and sensitive areas); Mean subject (or caregiver)-assessed LTS scores by visit.

Phase 3 OL-LTE study objective: To evaluate the efficacy of tapinarof cream, 1% over an extended period of time in subjects with AD. Endpoints: For subjects entering the study with a validated Investigator Global Assessment for Atopic Dermatitis (vIGA-AD) score of clear (0): Proportion of subjects who experience vIGA-AD≥2 at least 1 time during the study; Proportion of subjects who never experience vIGA-AD≥2 throughout the study; Time to first worsening (vIGA-AD≥2), defined as days from date of Baseline visit to the date of first occurrence of vIGA-AD≥2; Subjects who never experience vIGA-AD≥2 throughout the study will be censored at the date of their last vIGA-AD assessment. For subjects entering the study with a vIGA-AD score ≥1: Proportion of subjects who achieve vIGA-AD=0 at least 1 time during the study; Proportion of subjects who never achieve vIGA-AD=0 throughout the study; Time to first achieving a vIGA-AD score of 0, defined as days from date of Baseline visit to the date of first occurrence of vIGA-AD score of 0; Subjects who never achieve vIGA-AD=0 throughout the study will be censored at the date of their last vIGA-AD assessment. For subjects entering the study with a vIGA-AD score ≥2: Proportion of subjects who achieved vIGA-AD=0 or 1 at least 1 time during the study; Proportion of subjects who never achieve vIGA-AD=0 or 1 throughout the study. The following endpoints will be summarized over subjects in the intent-to-treat (ITT) population: Duration (days) of treatment periods, defined as time (days) from each treatment initiation/re-initiation to each subsequent disease clearance (vIGA-AD=0); Duration (days) of treatment-free intervals, defined as time (days) from each treatment discontinuation (vIGA-AD score of 0) to each subsequent treatment re-initiation (vIGA-AD≥2); vIGA-AD scores and the change from Baseline values by visit (observed case [OC] and last observation carried forward [LOCF]); Absolute value, change and percent change from Baseline in % BSA affected by visit (OC and LOCF); Absolute value, change and percent change from Baseline in BSA*vIGA-AD values by visit (OC and LOCF); Absolute value, change and percent change from Baseline in Eczema Area and Severity Index (EASI) score by visit (OC and LOCF); Proportion of subjects with ≥50% improvement in EASI score from Baseline by visit (OC and LOCF); Proportion of subjects with ≥75% improvement in EASI score from Baseline by visit (OC and LOCF); Proportion of subjects with ≥90% improvement in EASI score from Baseline by visit (OC and LOCF).

Phase 3 OL-LTE study objective: To describe the effect of tapinarof cream, 1% on AD symptom severity and the associated impact on daily activities and attitudes in subjects with AD. Endpoints: Mean change in Peak Pruritus-Numeric Rating Scale (PP-NRS) score from Baseline at each study visit; Proportion of subjects with a Baseline PP-NRS score ≥4 who achieve ≥4-point reduction in the PP-NRS from Baseline at each study visit; Proportion of subjects ≥12 years old with a Baseline PP-NRS score ≥4 who achieve ≥4-point reduction in the PP-NRS from Baseline at each study visit; Proportion of subjects <12 years old with a Baseline PP-NRS score ≥4 who achieve ≥4-point reduction in the PP-NRS from Baseline at each study visit; Mean change from Baseline in Dermatology Life Quality Index (DLQI), Children's Dermatology Life Quality Index (CDLQI), and Infant's Dermatitis Quality of Life Index (IDQOL) total and individual domain scores at each study visit; Mean change from Baseline in the total score of the Patient Oriented Eczema Measure (POEM) For Self-Completion (for subjects ≥12 years old) at each study visit; Mean change from Baseline in the total score of the POEM For Proxy-Completion (by caregiver for subjects <12 years old) at each study visit; Mean change from Baseline in EQ-5D-5L and EQ-SD-Y total and individual domain scores at each study visit; Mean change from Baseline in the total score of the Dermatitis Family Impact (DFI) at each study visit.

Study Design

Each Phase 3 pivotal study is a double-blind, randomized, vehicle-controlled, Phase 3, multicenter study to evaluate the efficacy and safety of topical tapinarof cream, 1% compared with vehicle cream in children and adult subjects with AD.

Following a 30-day screening period, eligible subjects will be randomized at a 2:1 ratio to receive QD treatment with tapinarof cream, 1% or vehicle cream for 8 weeks. Subjects will return to the clinic at Weeks 1, 2, 4, and 8 for efficacy and safety assessments. Additionally, subjects will be contacted by phone at Weeks 3 and 6 to assess AEs and concomitant medications, to review study drug administration instructions, and to confirm subject's continued participation in this study.

Study drug will be dispensed and applied during the clinic visits and will be administered at home between clinic visits as instructed by site personnel. Subjects or their caregivers will be instructed to apply study drug QD to all affected areas, including newly appearing lesions and lesions/areas that improve during the study. Subjects or their caregivers will apply sufficient study drug to cover completely each lesion with a thin layer of study drug and will record the time of study drug application and daily itch score (PP-NRS) in a daily diary provided by the study site. Subjects are allowed, but not required, to treat scalp lesions with study drug; however, efficacy analyses will not include assessment of AD in this area. Subjects and/or caregivers will be advised to maintain the approximate dosing time chosen at the beginning of the study for their full study participation. Nonmedicated emollients that do not contain salicylic acid may be used on nonlesional skin but the subject (or caregiver) should wait at least 30 minutes after applying study drug before applying nonmedicated emollients; emollients should not be applied to lesional skin during treatment. The same emollient should be used throughout the subject's participation in the study. At the phone contacts at Weeks 3 and 6, subjects or caregivers should be reminded to complete their daily diary and bring it with them to the next clinic visit.

Study drug application instructions will be reviewed at all post-randomization clinic visits and during any planned study phone calls. On clinic visit days, subjects and/or caregivers will be instructed/reminded on how to apply study drug (except during the final treatment/end-of-study visits). During the clinic visits, subjects or their caregivers will apply the daily dose of study drug while on-site under the supervision of site personnel after efficacy and safety assessments have been completed, with the exception of the LTS at some visits (as outlined in the Schedule of Assessments). The time of the dose application and assessments will depend on the time of the clinic visit. Therefore, the timing of the clinic visit may lead to a change in the subject's chosen dosing time for that day.

At the end of the 8 weeks of assessments in this study, subjects will have the option to enroll in an Open-Label, Long-Term Extension (OL-LTE) study for an additional 48 weeks. Subjects who complete Visit 6/Week 8 but choose not to participate in the OL-LTE study or who fail to qualify for participation in the OL LTE study will complete a Follow-up Visit (Visit 7/Week 9 visit) approximately 1 week after the end of treatment in this study. Subjects who withdraw from the study before Visit 6/Week 8 will complete an Early Termination Visit as their final visit and are not eligible for the OL-LTE study. The subjects who complete the Early Termination Visit must not complete a Follow-Up Visit.

Study duration for subjects who complete this Phase 3 pivotal study and who fail to qualify for participation in the OL-LTE study, or who qualify to participate in the OL-LTE study but elect not to enroll in that study is approximately 13 weeks in total. Study duration for subjects who complete this Phase 3 study and are eligible and decide to participate in the OL-LTE study is approximately 12 weeks in total.

Efficacy assessments will include vIGA AD score, % BSA affected, EASI, PP NRS, DLQI/CDLQI/IDQOL (depending on age), POEM For Self-Completion/POEM For Proxy-Completion (depending on age), EQ-5D-5L/EQ-5D-Y (depending on age), and DFI (if applicable). Safety assessments will include AEs, clinical laboratory tests, physical examination, vital signs, and ECGs (in a subset of subjects), and LTS. PK will be assessed in a subset of subjects at Week 4 and Week 8.

The Phase 3 OL-LTE study is an open-label, long-term multicenter, study to evaluate the safety and efficacy of topical tapinarof cream, 1% in subjects with AD. Subjects in this study will have either: a) completed treatment with tapinarof or vehicle in one of two Phase 3 pivotal safety and efficacy studies, DMVT-505-3101 or DMVT-505-3102, and rolled over into this study; b) completed treatment with tapinarof in the Phase 2 maximal use PK study, DMVT-505-2104, and rolled over into this study; or c) enrolled directly into this study. This study will consist of up to 48 weeks of treatment and a 1-week safety follow-up period.

At the completion of the Week 8 visit of study DMVT-505-3101 or study DMVT-505-3102 or the Day 28 visit of study DMVT-505-2104 (Baseline [Day 1] in this study), all eligible subjects will be offered enrollment in this OL-LTE study. Approximately 125 additional pediatric subjects ages 2 to <18 years who are not eligible for participation in the Phase 3 pivotal studies (DMVT-505-3101 or DMVT-505-3102) will be enrolled directly into this OL-LTE study. Study visits during the treatment period for all subjects will occur every 4 weeks (±3 days). Unscheduled visits may occur, as needed. Subjects who withdraw from the study before Week 48 will return to the study site for an Early Termination visit. The total duration of subject participation in this study will be approximately 49 weeks for rollover subjects (Baseline to Follow-Up) and approximately 53 weeks for direct-enrolling subjects (Screening to Follow-Up).

Rollover subjects in this study will begin treatment based on their vIGA-AD score from the final visit in one of the three aforementioned studies (DMVT-505-3101, DMVT-505-3102, or DMVT-505-2104). Subjects entering with a vIGA-AD≥1 will receive treatment with tapinarof cream, 1% QD until they achieve a vIGA-AD score of 0, at which time treatment will be discontinued and subjects monitored for maintenance of disease control (i.e., the extent of the remittive effect). If/when disease worsening occurs, as evidenced by a flare to vIGA-AD≥2, treatment will then be re-initiated and continued until a vIGA-AD of 0 is achieved. Subjects entering with a vIGA-AD of 0 will have treatment discontinued beginning at the Baseline visit and will be monitored for maintenance of the remittive effect. If/when disease worsening occurs, as evidenced by a vIGA-AD≥2, treatment will then be re-initiated and continued until a vIGA-AD of 0 is achieved. This treatment and re-treatment pattern of use will be continued until the end of the study (i.e., subjects may receive study treatment up until the Week 48 visit).

Subjects enrolling directly into this study will receive treatment QD with tapinarof cream, 1% beginning at Baseline and continue treatment until they achieve a vIGA-AD score of 0, at which time treatment will be discontinued and subjects monitored for maintenance of the remittive effect. If/when disease worsening occurs, as evidenced by a flare to vIGA-AD≥2, treatment will be re-initiated and continued until a vIGA-AD of 0 is achieved. This regimen of treatment and re-treatment will continue until the end of the study (i.e., subjects may receive study drug up until the Week 48 visit).

Study drug will be dispensed to subjects or their caregivers, applied during the clinic visits, and applied at home between clinic visits as instructed by site personnel. Subjects or their caregivers will be instructed to apply study drug QD to all affected areas, including newly appearing lesions and lesions/areas that improve during the study until a vIGA-AD of 0 is achieved. Once a vIGA-AD of 0 is achieved, the treatment period ends. If/when disease worsening occurs, and treatment is re-initiated at a vIGA-AD of ≥2, all lesions currently present and any new lesions that occur during the new treatment period should be treated. Subjects or their caregivers will apply sufficient study drug to cover each lesion completely with a thin layer of study drug and will record the time of study drug application and daily itch score (PP-NRS) in a daily diary provided by the study site. Subjects are allowed, but not required, to treat scalp lesions with study drug; however, efficacy analyses will not include assessment of AD on the scalp. At the first clinic visit, if applicable, subjects and/or caregivers will be instructed to maintain the approximate dosing time for the daily application of study drug. At the phone contact at Week 2, subjects or caregivers should be reminded to complete their daily diary and bring it with them to the next clinic visit.

Study drug application instructions will be reviewed at all post-randomization clinic visits and during any planned study phone calls. On clinic visit days, subjects and/or caregivers will be instructed/reminded on how to apply study drug (except during the final treatment/end-of-study visits). During the clinic visits, subjects or their caregivers will apply the daily dose of study drug while on-site under the supervision of site personnel, after efficacy and safety assessments have been completed (except for LTS at Week 4 through Week 44). The time of the dose application and assessments will depend on the time of the clinic visit. Therefore, the timing of the clinic visit may lead to a change in the subject's chosen dosing time for that day.

Safety assessments will include AEs, clinical laboratory tests, physical examination, vital signs, and LTS. Efficacy assessments will include vIGA-AD score, % BSA affected, EASI, PP-NRS, DLQI/CDLQI/IDQOL (depending on age), POEM For Self-Completion/POEM For Proxy-Completion (depending on age), EQ-5D-5L/EQ-5D-Y (depending on age), DFI (if applicable), and the Patient Satisfaction Questionnaire.

Treatment Groups and Duration

Each 8-week, Phase 3 pivotal study is a double-blind, vehicle controlled treatment study in which subjects will be randomized in a 2:1 ratio to receive QD treatment with either tapinarof cream, 1% or matching vehicle cream.

In order to complete the study, a subject must complete 8 weeks of study assessments. To be considered a “study completer” in terms of treatment period, a subject must complete ≥80.0% of the intended doses. The number of intended doses is defined as the Study Day of the subject's Week 8 visit minus 1 (e.g., if the subject's Week 8 visit occurs on Day 57, their number of intended doses is 56). To be eligible for the OL-LTE study, subjects must be a “study completer.” Subjects who complete the study will have the option to enroll in the Phase 3 OL-LTE study of 48 weeks in duration.

The end of study is defined as when the last active subject has completed the 8 weeks of assessments in this study and either enrolls in the OL-LTE study OR does not enroll in the OL-LTE study and completes the Week 9 (Follow up) Visit.

Subjects entering the Phase 3 OL-LTE study with a vIGA AD≥1 will receive treatment with tapinarof cream, 1% until they achieve a vIGA AD score of 0, at which time treatment will be discontinued and subjects monitored for remittive effect. If/when disease worsening occurs, as evidenced by a vIGA AD≥2, treatment will then be re initiated and continued until a vIGA AD of 0 is achieved. Subjects entering with a vIGA AD of 0 will have treatment discontinued beginning at the Baseline visit and will be monitored for remittive effect. If/when disease worsening occurs, as evidenced by a vIGA AD≥2, treatment will then be re-initiated and continued until a vIGA AD of 0 is achieved.

A subject will be considered to have completed the Phase 3 OL-LTE study when he/she completes all required procedures/visits for the 48 week treatment period and the Week 49 (Follow-Up) visit. The end of the study is defined as when the last active subject has completed the Follow-up Visit.

Type and Number of Subjects

Approximately 400 adult and pediatric subjects ages 2 years and above with AD will be enrolled in each Phase 3 pivotal study at approximately 60 study sites in the US and Canada. A minimum of approximately 15% of subjects will be enrolled into each of the following age groups: 2-6 years, 7-11 years, 12-17 years, 18 years and above. Adults 18 years and above will comprise a maximum of 20% of enrolled subjects.

Up to 961 pediatric and adult subjects with AD will be enrolled in the Phase 3 OL-LTE study at up to approximately 135 study sites in the US and Canada.

Inclusion Criteria

Each subject must meet all of the following criteria to be eligible to participate in the Phase 3 pivotal study:

Male and female subjects ages 2 years and above with clinical diagnosis of AD by Hanifin and Rajka criteria.

Subjects with AD covering ≥5% and ≤35% of the BSA. Scalp should be excluded from the BSA calculation to determine eligibility during Screening and at Baseline, and for all efficacy assessments. Subjects with disease only on palms and soles are not eligible.

A vIGA AD score of >3 at Screening and Baseline (pre-randomization).

An EASI score of >6 at Screening and Baseline (pre-randomization).

AD present for at least 6 months for ages 6 years old and above or 3 months for ages 2 to 5 years old, confirmed by prior medical documentation and/or according to the subject/caregiver report.

Female subjects of childbearing potential who are engaging in sexual activity that could lead to pregnancy should use one of the following acceptable birth control methods while on study and for 4 weeks after the last exposure to study drug. Acceptable contraception methods include intrauterine device, hormonal contraceptives, barrier method (e.g., condom or diaphragm), or surgical sterilization of male partner (vasectomy). Subjects who claim abstinence as their method of contraception are allowed provided they agree to use a barrier method (e.g., condom or diaphragm) should they become sexually active from Screening to 4 weeks after the last dose of study drug. Non-child-bearing potential is defined as: premenarchal; pre-menopausal females with a documented bilateral tubal ligation, bilateral oophorectomy, hysterectomy, or hysteroscopic sterilization; postmenopausal female with a cessation of menses for at least 12 months without an alternative medical cause; a blood sample with follicle stimulating hormone >40 mIU/mL is confirmatory in questionable cases.

Female subjects of childbearing potential must have a negative serum pregnancy test at Screening and a negative urine pregnancy test at Baseline (Day 1).

Subject, subject's parent(s), or legal representative must be capable of giving written informed consent/assent, which includes compliance with the requirements and restrictions listed in the consent/assent form; written informed consent must be obtained prior to any study related procedures.

Each subject must meet all of the following criteria to be eligible to participate in the Phase 3 OL-LTE study:

For Rollover Subjects Only: Met the criteria as a Study Completer in one of three parent studies (Phase 3 pivotal safety and efficacy study DMVT-505-3101, Phase 3 pivotal safety and efficacy study DMVT-505-3102, or Phase 2 maximal use PK study DMVT-505-2104) and was still receiving study drug at the last visit of one of these studies (to meet the criteria as a Study Completer in one of the three parent studies, a subject must have completed ≥80% of the intended doses in that study); Female subjects of childbearing potential must have a negative urine pregnancy test at Baseline (Day 1).

For Direct-Enrolling Subjects Only: Male and female subjects ages 2 years to <18 years at the time of consent with clinical diagnosis of AD by Hanifin and Rajka criteria; Subjects with a vIGA AD score of ≥3 and AD covering ≥40% of the BSA at Screening and Baseline (pre-randomization), or subjects with a vIGA AD score of 2 at Screening and Baseline (pre-randomization) regardless of BSA (Scalp should be excluded from the BSA calculation to determine eligibility during Screening and at Baseline, and for all efficacy assessments; Subjects must have screened for the DMVT-505-3101 or DMVT-505-3102 study and failed to meet BSA and/or vIGA AD™ eligibility criteria); Subjects with disease only on palms and soles are not eligible; AD present for at least 6 months for ages 6 years old and above or 3 months for ages 2 to 5 years old, confirmed by prior medical documentation and/or according to the subject/caregiver report; Female subjects of childbearing potential must have a negative serum pregnancy test at Screening and a negative urine pregnancy test at Baseline (Day 1).

For All Subjects (Rollover Subjects and Direct-Enrolling Subjects): Female subjects of childbearing potential who are engaging in sexual activity that could lead to pregnancy should use one of the following acceptable birth control methods while on study and for 4 weeks after the last exposure to study drug. Acceptable contraception methods include intrauterine device, hormonal contraceptives, barrier method (e.g., condom or diaphragm), or surgical sterilization of male partner (vasectomy). Subjects who claim abstinence as their method of contraception are allowed provided they agree to use a barrier method (e.g., condom or diaphragm) should they become sexually active from Baseline to 4 weeks after the last dose of study drug. Non-child-bearing potential is defined as: premenarchal; pre-menopausal females with a documented bilateral tubal ligation, bilateral oophorectomy, hysterectomy, or hysteroscopic sterilization; postmenopausal female with a cessation of menses for at least 12 months without an alternative medical cause; a blood sample with follicle stimulating hormone >40 mIU/mL is confirmatory in questionable cases; Subject, subject's parent(s), or legal representative must be capable of giving written informed consent/assent, which includes compliance with the requirements and restrictions listed in the consent/assent form; written informed consent must be obtained prior to any study related procedures.

Exclusion Criteria

A subject who meets any of the following criteria will be excluded and considered ineligible for participation in the Phase 3 pivotal study:

Concurrent conditions: Immunocompromised (e.g., lymphoma, acquired immunodeficiency syndrome) or history or evidence of active or latent tuberculosis or human immunodeficiency virus antibody as documented by medical history and/or according to the subject/caregiver report; Chronic or acute systemic infection requiring treatment with antiparasitics, or antiprotozoals, within 4 weeks prior to the Baseline visit; Chronic or acute systemic bacterial infection requiring treatment with systemic antibiotics within one week prior to the Baseline visit; Chronic or acute superficial fungal infection requiring treatment with systemic antifungals within one week prior to the Baseline visit; Acute active bacterial, fungal, or viral (herpes simplex, herpes zoster, chicken pox) skin infection within 1 week prior to the Baseline visit; the condition should be completely resolved one week prior to Baseline Visit; Significant dermatologic or inflammatory condition other than AD that, in the Investigator's opinion, would make it difficult to interpret data or assessments during the study (for example, subjects with an active skin condition such as Kaposi's varicelliform eruption, scabies, molluscum contagiosum, impetigo, psoriasis, severe acne, connective tissue disorder, or Netherton's syndrome, or any other concurrent active disease); Concurrent skin lesions in the treatment area or pruritus due to conditions other than AD that, in the opinion of the Investigator, would either interfere with study evaluations or affect the safety of the subject; Screening alanine aminotransferase (ALT) or aspartate aminotransferase (AST)≥2.0× the upper limit of normal (ULN); Screening total bilirubin >1.5×ULN; total bilirubin >1.5×ULN is acceptable if bilirubin is fractionated and direct bilirubin <35%; Current or chronic history of liver disease, known hepatic or biliary abnormalities (with the exception of Gilbert's syndrome or asymptomatic gallstones), presence of hepatitis B surface antigen (HBsAg), or positive hepatitis C antibody test result, or presence of anti-hepatitis B core antigen (anti-HBc). Subjects having a negative HBsAg and a positive anti-HBc may enroll if they have a positive anti-hepatitis B surface antigen demonstrating natural immunity; Subjects with a history of hepatitis C virus infection who were medically cured and have an undetectable viral load are eligible to enroll; Subjects with a history of stable non-alcoholic fatty liver disease without evidence of active inflammation (elevated ALT/AST≥2.0×ULN) or cirrhosis are eligible to enroll; Current or a history of cancer within 5 years except for adequately treated cutaneous basal cell carcinoma, squamous cell carcinoma or carcinoma in situ of the cervix (surgical excision or electrodessication and curettage); Subjects who would not be considered suitable for topical therapy (e.g., those with extensive disease involvement over a large BSA who would be candidates for systemic therapy); Use of any prohibited medication or procedure within the indicated period before the Baseline visit.

Prohibited concomitant medications, therapy, etc. during the defined period are as listed below. If a subject requires any of these medications throughout the study period, he/she may be excluded from or discontinued from the study, at the discretion of the Investigator and Medical Monitor.

From 4 months prior to Baseline until the completion of the Follow-up visit or study discontinuation: DUPIXENT (dupilumab) injection; Any monoclonal antibody product that becomes approved for AD during the course of the trial.

From 28 days prior to Baseline until the completion of the Follow-up visit or discontinuation: Oral, injectable, and suppository preparations of corticosteroids; Eye drops and nasal preparations are allowed; Inhaled preparations are allowed when used for a stable condition and stable dose for >28 days before Screening and are continued at the same dose throughout the study. Oral preparations and injections of immunosuppressants (cyclosporine, methotrexate, azathioprine, tacrolimus, Janus kinase inhibitors, etc.); Excessive sun exposure, tanning booth, other ultraviolet light source and phototherapy including psoralen and ultraviolet A therapy or is unwilling to minimize natural and artificial sunlight exposure; Treatment with antivirals with the exception of short-term treatment for acute upper respiratory viral infections (i.e., influenza) or viral suppressive therapy for a history of recurrent herpes labialis or genital herpes.

From 14 days prior to Baseline until the completion of the Follow-up visit or discontinuation: EUCRISA (crisaborole) and any other PDE4 inhibitor; Tacrolimus ointment and pimecrolimus cream; Topical corticosteroids that are classified as medium or high potency (e.g., fluocinonide, triamcinolone acetonide) or super-high potency (e.g., clobetasol propionate); Eye drops and nasal preparations are allowed; Coal tar products (on the body); If subject chooses to treat scalp with study drug, then coal tar products are prohibited for use on the scalp; Over the counter or herbal medicines for AD (topical and oral preparations); If subjects are using emollients, they may continue to use the same emollient on nonlesional skin during the study; Emollients containing salicylic acid are prohibited during the study.

From 7 days prior to Baseline until the completion of the Follow-up visit or discontinuation: Topical corticosteroids that are classified as low potency (e.g., desonide, hydrocortisone); Oral, injectable, or intravenous antibiotics or antifungal medications; Topical doxepin, topical gentamicin, or topical neomycin sulfate; Oral doxepin is allowed for treatment of depression if subject has been on a stable dose (4 weeks) at Screening; Topical products containing urea, except for the treatment of follicular events; Antihistamines/antiallergics (oral, topical and injections): diphenhydramine, chlorpheniramine maleate, hydroxyzine; The following antihistamines are allowed from Screening throughout the treatment period: loratadine, fexofenadine hydrochloride, cetirizine hydrochloride; Subjects are allowed to switch from non-allowed antihistamines to allowed antihistamines during Screening but must be on a stable dose for 7 days prior to Baseline; The subject has received an investigational product within the following time period prior to the first dosing day in the current study: 30 days or 5 half-lives of the investigational product (whichever is longer).

Additional exclusion criteria are a history of or ongoing serious illness or medical, physical, or psychiatric condition(s) that, in the Investigator's opinion, may interfere with the subject's participation in the study, interpretation of results, or ability to understand and give informed consent; Pregnant females as determined by positive serum (Screening) or urine (Baseline) human chorionic gonadotropin test; Lactating females; History of sensitivity to the study medications, or components thereof or a history of drug or other allergy that, in the opinion of the Investigator or Medical Monitor, contraindicates their participation; Previous known participation in a clinical study with tapinarof (previously known as GSK2894512 and WBI-1001).

A subject who meets any of the following criteria will be excluded and considered ineligible for participation in the Phase 3 OL-LTE study:

For Rollover Subjects Only: Subjects who were not receiving study drug at the time of the last visit in the parent study (DMVT-505-3101, DMVT-505-3102, or DMVT-505-2104); Used a prohibited concomitant product or procedure to treat AD during the parent study; Had an SAE that was related to treatment or experienced an AE that led to permanent discontinuation of treatment in the parent study; Pregnant females as determined by positive urine human chorionic gonadotropin test at Baseline.

For Direct-Enrolling Subjects Only: Concurrent conditions: Immunocompromised (e.g., lymphoma, acquired immunodeficiency syndrome) or medical history of positive human immunodeficiency virus antibody at Screening; Chronic or acute systemic infection requiring treatment with antiparasitics or antiprotozoals, within 4 weeks prior to the Baseline visit; Chronic or acute systemic bacterial infection requiring treatment with systemic antibiotics within one week prior to the Baseline visit; Chronic or acute superficial fungal infection requiring treatment with systemic antifungals within one week prior to the Baseline visit; Acute active bacterial, fungal, or viral (herpes simplex, herpes zoster, chicken pox) skin infection within 1 week prior to the Baseline visit; the condition should be completely resolved one week prior to Baseline Visit; Significant dermatologic or inflammatory condition other than AD that, in the Investigator's opinion, would make it difficult to interpret data or assessments during the study (for example, subjects with an active skin condition such as Kaposi's varicelliform eruption, scabies, molluscum contagiosum, impetigo, psoriasis, severe acne, connective tissue disorder, or Netherton's syndrome, or any other concurrent active disease); Concurrent skin lesions in the treatment area or pruritus due to conditions other than AD that, in the opinion of the Investigator, would either interfere with study evaluations or affect the safety of the subject; Screening alanine aminotransferase (ALT) or aspartate aminotransferase (AST)≥2.0× the upper limit of normal (ULN); Screening total bilirubin >1.5×ULN; total bilirubin >1.5×ULN is acceptable if bilirubin is fractionated and direct bilirubin <35%; Current or chronic history of liver disease, known hepatic or biliary abnormalities (with the exception of Gilbert's syndrome or asymptomatic gallstones), presence of hepatitis B surface antigen (HBsAg), or positive hepatitis C antibody test result, or presence of anti-hepatitis B core antigen (anti-HBc) (Subjects having a negative HBsAg and a positive anti HBc may enroll if they have a positive anti-hepatitis B surface antigen demonstrating natural immunity. Subjects with a history of hepatitis C virus infection who were medically cured and have an undetectable viral load are eligible to enroll. Subjects with a history of stable non-alcoholic fatty liver disease without evidence of active inflammation (elevated ALT/AST≥2.0×ULN) or cirrhosis are eligible to enroll); Current or a history of cancer within 5 years except for adequately treated skin basal cell carcinoma, cutaneous squamous cell carcinoma or carcinoma in situ of the cervix (surgical excision or electrodessication and curettage); Pregnant females as determined by positive serum (Screening) or urine (Baseline) human chorionic gonadotropin test; Lactating females; Previous known participation in a clinical study with tapinarof (previously known as GSK2894512 and WBI-1001); Use of any prohibited medication or procedure within the indicated period before the Baseline visit. Prohibited concomitant medications, therapy, etc. during the defined period are as listed below. If a subject requires any of these medications throughout the study period, he/she may be excluded from or discontinued from the study, at the discretion of the Investigator and Medical Monitor.

From 4 months prior to Baseline: DUPIXENT (dupilumab) injection; Any monoclonal antibody product that becomes approved for AD during the course of the trial.

From 28 days prior to Baseline: Oral, injectable, and suppository preparations of corticosteroids; Eye drops and nasal preparations are allowed; Inhaled preparations are allowed when used for a stable condition and stable dose for >28 days before Screening and are continued at the same dose throughout the study; Oral preparations and injections of immunosuppressants (cyclosporine, methotrexate, azathioprine, tacrolimus, Janus kinase [JAK] inhibitors, etc.); Excessive sun exposure, tanning booth, other ultraviolet light source and phototherapy including psoralen and ultraviolet A therapy or is unwilling to minimize natural and artificial sunlight exposure; Treatment with antivirals with the exception of short-term treatment for acute upper respiratory viral infections (i.e., influenza) or viral suppressive therapy for a history of recurrent herpes labialis or genital herpes.

From 14 days prior to Baseline: EUCRISA® (crisaborole) and any other PDE4 inhibitor; Tacrolimus ointment and pimecrolimus cream; Topical JAK inhibitors; Topical corticosteroids that are classified as medium or high potency (e.g., fluocinonide, triamcinolone acetonide) or super-high potency (e.g., clobetasol propionate). Eye drops and nasal preparations are allowed; Over the counter or herbal medicines for AD (topical and oral preparations). If subjects are using emollients, they may continue to use the same emollient on nonlesional skin during the study. Emollients containing salicylic acid are prohibited during the study.

From 7 days prior to Baseline: Topical corticosteroids that are classified as low potency (e.g., desonide, hydrocortisone); Oral, injectable, or intravenous antibiotics or antifungal medications; Topical doxepin, topical gentamicin, or topical neomycin sulfate; Oral doxepin is allowed for treatment of depression if subject has been on a stable dose (4 weeks) at Screening; Topical products containing urea, except for the treatment of follicular events; Antihistamines/antiallergics (oral, topical and injections): diphenhydramine, chlorpheniramine maleate, hydroxyzine; For direct-enrolling subjects, the following antihistamines are allowed from Screening throughout the treatment period: loratadine, fexofenadine hydrochloride, cetirizine hydrochloride. Subjects are allowed to switch from non-allowed antihistamines to allowed antihistamines during Screening but must be on a stable dose for 7 days prior to Baseline.

The subject has received an investigational product within the following time period prior to the first dosing day in the current study: Minimum of 30 days or 5 half-lives of the investigational product (whichever is longer).

For All Subjects (Rollover Subjects and Direct-Enrolling Subjects): A history of or ongoing serious illness or medical, physical, or psychiatric condition(s) that, in the Investigator's opinion, may interfere with the subject's participation in the study, interpretation of results, safety of the subject or ability to understand and give informed consent; Known hypersensitivity to tapinarof or excipients.

Study Drug

The descriptions of the study drugs, tapinarof cream, 1% and the vehicle cream, are presented in Table 23.

TABLE 23
Tapinarof and Vehicle Cream.

| | Tapinarof (DMVT-505, | |
|---|---|---|
| Study Drug | Formulation F) | Vehicle Cream |
| Physical description | White to off-white cream | White to off-white |
| | | cream |
| Unit dose strength/how | 1% w/w | NA/30-gram tube |
| supplied | (10 mg/gram)/30-gram tube | |
| Route of | Topical/8 weeks (or 48 | Topical/8 weeks |
| administration/duration | weeks for OL-LTE study) | |
| Dosing instructions | QD topical application of | QD topical |
| | thin layer to affected areas | application of thin |
| | | layer to affected |
| | | areas |
| Manufacturer | GlaxoSmithKline (Barnard | GlaxoSmithKline |
| | Castle, UK) | (Barnard Castle, |
| | | UK) |

NA = not applicable;
QD = once daily;
w/w = weight/weight

The excipients included in tapinarof cream and vehicle cream are purified water, medium chain triglycerides, propylene glycol, emulsifying wax, diethylene glycol monoethyl ether, polyoxyl stearyl ether-2, polysorbate 80, polyoxyl stearyl ether-20, benzoic acid, sodium citrate, butylated hydroxytoluene, edetate disodium and citric acid monohydrate.

Administration of Study Drug

Study drug will be dispensed to subjects or their caregivers at the clinical site in appropriately labeled tubes. Subjects or caregivers will take the tubes home and self-administer study drug (or have caregiver apply if necessary) to affected areas QD, except on clinic visit days when study drug is applied under supervision at the site.

Subjects of each Phase 3 pivotal study will be instructed to apply study drug as follows:

QD application to affected areas; subjects or their caregivers are advised to choose the application time they prefer and to apply the study drug at that approximate time each day of study participation. Subjects should avoid dosing around midnight to avoid potentially dosing twice in one calendar day.

If a subject misses a daily dose, it will be recorded as a protocol deviation. The subject should continue dosing the next day and should not apply more than once daily to make up for the missed dose on the previous day. If a dose is missed, the missed dose and the reason for the missed dose should be recorded in the daily diary as such. Itch rating can still be recorded in the diary even if a daily dose was missed.

Study drug should be applied to dry, clean skin; Study drug may be applied to skin around the eye but avoid direct contact with the eye—study drug is not for ophthalmic use; Wash hands after application, unless treating lesions on the hands; Study drug should be applied to all lesions, including newly appearing lesions and lesions that have improved or resolved during the study. A body diagram identifying locations of lesions may be provided to the subject and/or caregiver; Subjects are allowed, but not required, to treat scalp lesions with study drug; however, efficacy analyses will not include assessment of AD in this area; If there is residual cream visible on the disease-affected lesional skin, then the subject or caregiver should be instructed to continue to lightly rub the cream into the skin until it is no longer visible; If study drug is applied to the subject by another person, that person should thoroughly wash his/her hands after application; When dosing at home subjects or caregiver should record the time of study drug application in the daily diary; Itch rating should also be recorded in the diary; On clinic visit days, study drug should be applied in the clinic under the supervision of site personnel and after safety and efficacy assessments have been completed (except for subject and Investigator LTS at Weeks 1, 2 and 4); The time of the dose and assessments on clinic visit days will depend on the time of the clinic visit. Therefore, the timing of the clinic visit may differ from the subject's chosen dosing time. The intention is to allow flexibility to accommodate subjects' schedules.

Nonmedicated emollients that do not contain salicylic acid may be used on nonlesional skin but the subject (or caregiver) should wait at least 30 minutes after applying study drug before applying nonmedicated emollients; emollients should not be applied to lesional skin during treatment. The same emollient should be used throughout the subject's participation in the study. Subjects and/or caregivers will be instructed/reminded on how to apply study drug at each clinic visit (except during the final treatment visit).

Subjects entering the Phase 3 OL-LTE study with a vIGA-AD™≥1 (including all subjects enrolling directly into the study) will receive treatment QD with tapinarof cream, 1% until they achieve a vIGA-AD score of 0, at which time treatment will be discontinued and subjects monitored for maintenance of disease control (i.e. the extent of the remittive effect). If/when disease worsening occurs, as evidenced by a flare to vIGA-AD≥2, treatment will be re-initiated and continued until a vIGA AD of 0 is achieved. Subjects entering with a vIGA-AD of 0 will have treatment discontinued beginning at the Baseline visit and will be monitored for maintenance of the remittive effect. If/when disease worsening occurs, as evidenced by a flare to vIGA-AD≥2, treatment will be re-initiated and continued until a vIGA-AD of 0 is achieve. Once a vIGA-AD of 0 is achieved, the treatment period ends. If/when disease worsening occurs, and treatment is re-initiated at a vIGA-AD of ≥2, all lesions currently present and any new lesions that occur during the new treatment period should be treated. This treatment and re-treatment pattern of use will be continued until the end of the study (i.e., subjects may receive study treatment up until the Week 48 visit).

Subjects who experience suspected disease worsening between scheduled study visits may contact the study site to arrange an unscheduled visit. Upon confirmation of disease worsening, subjects will restart treatment with study drug.

If disease worsening is suspected between scheduled study visits, an unscheduled visit may be performed. If disease worsening (i.e., vIGA-AD≥2) is not confirmed, the subject will continue to be assessed at routine, scheduled visits.

For each confirmed episode of disease worsening (i.e., vIGA-AD≥2) during the study (at either a scheduled or unscheduled visit), a treatment course of tapinarof cream, 1% QD will be initiated and will continue until the subject achieves a vIGA-AD of 0, as assessed at scheduled visits, approximately every 4 weeks beginning at Baseline.

Subjects of the Phase 3 OL-LTE study will be instructed to apply study drug as follows:

QD application to affected areas; subjects or their caregivers are advised to choose the application time they prefer and to apply the study drug at that approximate time each day of study participation. Subjects should avoid dosing around midnight to avoid potentially dosing twice in one calendar day.

If a subject misses a daily dose, it will be recorded as a protocol deviation. The subject should continue dosing the next day and should not apply more than once daily to make up for the missed dose on the previous day. If a dose is missed, the missed dose and the reason for the missed dose should be recorded in the daily diary as such. Itch rating can still be recorded in the diary even if a daily dose was missed.

Study drug should be applied to dry, clean skin; Study drug may be applied to skin around the eye but avoid direct contact with the eye—study drug is not for ophthalmic use; Wash hands after application, unless treating lesions on the hands.

Study drug should be applied to all lesions, including newly appearing lesions and lesions/areas that improve during the study until a vIGA-AD of 0 is achieved. Once a vIGA-AD of 0 is achieved, the treatment period ends. If/when disease worsening occurs, and treatment is re-initiated at a vIGA-AD of ≥2, all lesions currently present and any new lesions that occur during the new treatment period should be treated. A body diagram identifying locations of lesions may be provided to the subject and/or caregiver.

Subjects are allowed, but not required, to treat scalp lesions with study drug; however, efficacy analyses will not include assessment of AD in this area; If there is residual cream visible on the disease-affected lesional skin, then the subject or caregiver should be instructed to continue to lightly rub the cream into the skin until it is no longer visible; If study drug is applied to the subject by another person, that person should thoroughly wash his/her hands after application; When dosing at home subjects or caregiver should record the time of study drug application in the daily diary. Itch rating should also be recorded in the diary; On clinic visit days, study drug should be applied in the clinic under the supervision of site personnel and after safety and efficacy assessments have been completed (except for subject [or caregiver]- and Investigator-assessed LTS); The time of the dose and assessments on clinic visit days will depend on the time of the clinic visit. Therefore, the timing of the clinic visit may differ from the subject's chosen dosing time. The intention is to allow flexibility to accommodate subjects' schedules.

Nonmedicated emollients that do not contain salicylic acid may be used on nonlesional skin but the subject (or caregiver) should wait at least 30 minutes after applying study drug before applying nonmedicated emollients; emollients should not be applied to lesional skin during treatment. The same emollient should be used throughout the subject's participation in the study.

Randomization/Treatment Assignment

For the double-blind, vehicle-controlled phase of the study (Phase 3 pivotal study), subjects will be randomized at a ratio of 2:1 to receive tapinarof cream, 1% or vehicle cream as follows:

TABLE 24
Randomization in Double-Blind, Vehicle-Controlled Phase

| Regimen | Number of Subjects |
|---|---|
| Tapinarof cream, 1% QD for 8 weeks | Approximately 267 Subjects |
| Vehicle cream QD for 8 weeks | Approximately 133 Subjects |

QD = once daily

Randomization will be stratified by Baseline vIGA AD score so that subjects with severe AD (vIGA AD score of 4) will be limited to approximately 10% each of the total randomized population, and the majority of the enrolled subjects (approximately 90%) will have a vIGA AD score of 3, signifying moderate disease.

Randomization will also be stratified by age so that a minimum of approximately 15% of subjects will be enrolled into each of the following age groups: 2-6 years, 7-11 years, 12-17 years, 18 years and above. Adults 18 years and above will comprise a maximum of 20% of enrolled subjects.

The randomization lists will be generated using a validated system, which involves a pseudo random number generator so that the resulting treatment will be both reproducible and non-predictable. Access to the codes will be controlled and documented.

Efficacy Assessments: Phase 3 pivotal studies

To minimize inter-observer variability, Investigators and evaluators/raters will be trained on each of the required assessments during an Investigator meeting, site initiation visit, and/or utilizing online assessments before enrolling subjects at their study site. Only trained evaluators/raters are permitted to perform the efficacy assessments. To the fullest extent possible, the same Investigator (or designated evaluator/rater) will perform all efficacy assessments for an individual subject throughout the study. If it is not possible for the same evaluator/rater to continue performing assessments, it is recommended that the primary and subsequent evaluator/rater both examine and discuss their respective scoring during at least 1 visit.

Validated Investigator Global Assessment of Atopic Dermatitis: The vIGA-AD of disease severity will be assessed at every clinic visit. The vIGA-AD is a global assessment of the current state of the disease. It is a 5-point morphological assessment of overall disease severity (scalp excluded) and will be determined according to the categories described in Table 25. To be eligible, subjects must have a vIGA-AD score of 3 or 4 at Screening and the Baseline visit (Day 1). Eli Lilly and Company developed the vIGA-AD scale for use in clinical trials.

TABLE 25
Validated Investigator Global Assessment Scale for Atopic Dermatitis

| Score | Category | Definition |
|---|---|---|
| 0 | Clear | No inflammatory signs of atopic dermatitis (no erythema, no |
| | | induration/papulation, no lichenification, no oozing/crusting). |
| | | Post-inflammatory hyperpigmentation and/or hypopigmentation |
| | | may be present. |
| 1 | Almost clear | Barely perceptible erythema, barely perceptible |
| | | induration/papulation, and/or minimal lichenification. No oozing |
| | | or crusting. |
| 2 | Mild | Slight but definite erythema (pink), slight but definite |
| | | induration/papulation, and/or slight but definite lichenification. |
| | | No oozing or crusting |
| 3 | Moderate | Clearly perceptible erythema (dull red), clearly perceptible |
| | | induration/papulation, and/or clearly perceptible lichenification. |
| | | Oozing and crusting may be present. |
| 4 | Severe | Marked erythema (deep or bright red), marked |
| | | induration/papulation, and/or marked lichenification. Disease is |
| | | widespread in extent. Oozing or crusting may be present. |

Body Surface Area Affected: The assessment of the % BSA affected is an estimate of the percentage of total involved skin with AD. For the purpose of clinical estimation, the total palmar surface of the subject's palm and digits may be assumed to be approximately equivalent to 1% BSA. The % BSA affected by AD will be evaluated from 0% to approximately 100% (scalp excluded). Percentage BSA is a static assessment made without reference to previous scores.

Eczema Area and Severity Index: The EASI will be assessed at every clinic visit. It quantifies the severity of a subject's AD based on both lesion severity and the % BSA affected. The subject's scalp is excluded from this assessment. The EASI is a composite score ranging from 0 to 72 that takes into account the degree of erythema, edema/papulation, excoriation, and lichenification (each scored from 0 to 3 separately) for each of four body regions, with adjustment for the % BSA involved for each body region relative to the whole body. The EASI score will be calculated in the CRF based on the subject's age, the rating scores for each region, and the number of handprints involved for each region.

Dermatology Life Quality Index, Children's Dermatology Life Quality Index, and Infant's Dermatitis Quality of Life Index: The IDQOL will be used for ages 2 to <4 years and completed by the caregiver. The CDLQI will be used for ages 4 to <16 years and completed by the subject with the help of the caregiver, as necessary. The DLQI will be used for ages 16 years and above and completed by the subject. A subject will continue to use the same questionnaire throughout the study, regardless of a change in age during their participation. The DLQI, CDLQI, and IDQOL are simple dermatology-specific 10 question validated questionnaires to assess the impact of the disease on a subject's quality of life for adults, children, and infants, respectively (Cardiff University, Department of Dermatology, Quality of Life Questionnaires). The DLQI has become an important outcome measure in dermatology clinical trials and is the most frequently used instrument in studies of randomized controlled trials in dermatology. The DLQI can be analyzed as a total score (where a higher score indicates greater impairment in quality of life) and can also be scored for the following dimensions: Symptoms and Feelings (items 1 and 2), Daily Activities (items 3 and 4), Leisure (items 5 and 6), Work and School (item 7), Personal Relationships (items 8 and 9), and Treatment (item 10).

Patient Oriented Eczema Measures For Self-Completion and For Proxy-Completion: The POEM For Self-Completion and POEM For Proxy-Completion are tools used for monitoring AD severity. They focus on the illness as experienced by the patients. For subjects ages 2 to <12 years, the POEM For Proxy-Completion will be completed by the caregiver. For subjects ages ≥12 years, the POEM For Self-Completion will be completed by the subject.

Peak Pruritus Numeric Rating Scale: The PP-NRS is a scale used to quickly assess itch/pruritus severity over a 24-hour period. The subject or caregiver will utilize the scale to assess peak pruritus QD and record the results in their daily diaries. The itch rating can be done before or after study drug administration since it should reflect the past 24 hours. On clinic visit days, the PP-NRS will be assessed in the clinic. For subjects ages 2 to <12 years, the PP-NRS will be completed by the caregiver. For subjects ages ≥12 years, the PP-NRS will be completed by the subject.

EQ-5D-5L and EQ-SD-Y: The EQ-5D-5L was introduced by the EuroQol Group in 2009 to improve the instrument's sensitivity and to reduce ceiling effects, as compared to the EQ-5D-3L. The EQ-5D-5L essentially consists of 2 pages: the EQ-5D descriptive system and the EQ visual analogue scale. The descriptive system comprises five dimensions: mobility, self-care, usual activities, pain/discomfort and anxiety/depression. Each dimension has 5 levels: no problems, slight problems, moderate problems, severe problems and extreme problems. The subject is asked to indicate his/her health state by ticking the box next to the most appropriate statement in each of the five dimensions. This decision results in a 1-digit number that expresses the level selected for that dimension. The digits for the five dimensions can be combined into a 5-digit number that describes the patient's health state. The EQ visual analogue scale records the patient's self-rated health on a vertical visual analogue scale, where the endpoints are labelled ‘The best health you can imagine’ and ‘The worst health you can imagine’. The visual analogue scale can be used as a quantitative measure of health outcome that reflects the patient's own judgement. The EQ-5D-5L will be used in subjects ≥16 years and completed by the subject. The child-friendly EQ-5D version (EQ-SD-Y) was introduced by the EuroQol Group in 2009 as a more comprehensible instrument suitable for children and adolescents. The EQ-SD-Y is based on the EQ-5D-3L and essentially consist of 2 pages: the EQ-5D descriptive system and the EQ visual analogue scale. The EQ-SD-Y descriptive system comprises the following five dimensions: mobility, looking after myself, doing usual activities, having pain or discomfort and feeling worried, sad or unhappy. Each dimension has 3 levels: no problems, some problems and a lot of problems. The younger patient is asked to indicate his/her health state by ticking the box next to the most appropriate statement in each of the five dimensions. This decision results in a 1-digit number that expresses the level selected for that dimension. The digits for the five dimensions can be combined into a 5-digit number that describes the younger patient's health state. The EQ visual analogue scale records the younger patient's self-rated health on a vertical visual analogue scale where the endpoints are labelled “The best health you can imagine” and “The worst health you can imagine”. The visual analogue scale can be used as a quantitative measure of health outcome that reflects the younger patient's own judgement. The EQ-SD-Y will be used in subjects ≥8 to <16 years and completed by the subject.

Dermatitis Family Impact Questionnaire: The DFI is a questionnaire that measure how much having a child with AD affects the quality of life of other members of the family. It is a 10-question tool that will be completed by the caregiver age 16 or over who has a pediatric subject <16 years old in the study. The same caregiver should be used to complete each questionnaire.

Optional Clinical Photography: Clinical photography may be performed in a subgroup of subjects at selected study sites. Informed consent/assent and photographic release will be required. The photographs may not be referred to by the Investigator at any subsequent study visit for the purposes of grading. Photographs will be taken of a representative area of the subject's disease area at the time points specified in the Schedule of Assessments. Photographs of the selected skin area will be taken in a standardized fashion (i.e., same camera, angle, background, distance).

Efficacy Assessments: Phase 3 OL-LTE study

To minimize inter observer variability, Investigators and/or evaluators/raters will be trained on each of the required assessments during an Investigator meeting, site initiation visit, and/or utilizing online assessments before enrolling subjects at their study site. Only trained evaluators/raters are permitted to perform the efficacy assessments. To the fullest extent possible, the same Investigator (or designated evaluator/rater) will perform all efficacy assessments for an individual subject throughout the study. If it is not possible for the same evaluator/rater to continue performing assessments, it is recommended that the primary and subsequent evaluator/rater both examine and discuss their respective scoring during at least 1 visit.

Validated Investigator Global Assessment of Atopic Dermatitis: The vIGA-AD of disease severity will be assessed at every clinic visit. The vIGA-AD is a global assessment of the current state of the disease. It is a 5-point morphological assessment of overall disease severity (scalp excluded) and will be determined according to the categories described in Table 25 above. Eli Lilly and Company developed the vIGA-AD scale for use in clinical trials.

Body Surface Area Affected: The assessment of the % BSA affected is an estimate of the percentage of total involved skin with AD. For the purpose of clinical estimation, the total palmar surface of the subject's palm and digits may be assumed to be approximately equivalent to 1% BSA. The % BSA affected by AD will be evaluated from 0% to approximately 100% (scalp excluded). Percentage BSA is a static assessment made without reference to previous scores.

Eczema Area and Severity Index: The EASI will be assessed at every clinic visit. It quantifies the severity of a subject's AD based on both lesion severity and the % BSA affected. The subject's scalp is excluded from this assessment. The EASI is a composite score ranging from 0 to 72 that takes into account the degree of erythema, edema/papulation, excoriation, and lichenification (each scored from 0 to 3 separately) for each of four body regions, with adjustment for the % BSA involved for each body region relative to the whole body. The EASI score will be calculated in the CRF based on the subject's age, the rating scores for each region, and the number of handprints involved for each region.

Dermatology Life Quality Index, Children's Dermatology Life Quality Index, and Infant's Dermatitis Quality of Life Index: The IDQOL will be used for ages 2 to <4 years and completed by the caregiver. The CDLQI will be used for ages 4 to <16 years and completed by the subject with the help of the caregiver, as necessary. The DLQI will be used for ages 16 years and above and completed by the subject. For subjects rolling over from DMVT-505-3101 or DMVT-505-3102, the DLQI, CDLQI, or IDQOL completed at the final visit of the parent study will be used as the Baseline value for this study. For subjects rolling over from DMVT-505-2104 or subjects enrolling directly into DMVT-505-3103, the DLQI, CDLQI, or IDQOL should be completed pre-dose at Baseline. The DLQI, CDLQI, and IDQOL are simple dermatology-specific 10-question validated questionnaires to assess the impact of the disease on a subject's quality of life for adults, children, and infants, respectively (Cardiff University, Department of Dermatology, Quality of Life Questionnaires). The DLQI has become an important outcome measure in dermatology clinical trials and is the most frequently used instrument in studies of randomized controlled trials in dermatology. The DLQI can be analyzed as a total score (where a higher score indicates greater impairment in quality of life) and can also be scored for the following dimensions: Symptoms and Feelings (items 1 and 2), Daily Activities (items 3 and 4), Leisure (items 5 and 6), Work and School (item 7), Personal Relationships (items 8 and 9), and Treatment (item 10).

Patient Oriented Eczema Measures For Self-Completion and For Proxy Completion: The POEM For Self-Completion and POEM For Proxy-Completion are tools used for monitoring AD severity. They focus on the illness as experienced by the patients. For subjects ages 2 to <12 years, the POEM For Proxy-Completion will be completed by the caregiver. For subjects ages ≥12 years, the POEM For Self-Completion will be completed by the subject. For subjects rolling over from DMVT-505-3101 or DMVT-505-3102, the POEM For Self-Completion or POEM For Proxy Completion completed at the final visit of the parent study will be used as the Baseline value for this study. For subjects rolling over from DMVT-505-2104 or subjects enrolling directly into DMVT-505-3103, the POEM For Self-Completion or POEM For Proxy Completion should be completed pre-dose at Baseline.

Peak Pruritus-Numeric Rating Scale: The PP-NRS is a scale used to quickly assess itch/pruritus severity over a 24-hour period. The subject or caregiver will utilize the scale to assess peak pruritus QD and record the results in their daily diaries. The itch rating can be done before or after study drug administration since it should reflect the past 24 hours. On clinic visit days, the PP-NRS will be assessed in the clinic. PP-NRS should be completed every day, even if subjects are not actively applying study drug. For subjects ages 2 to <12 years, the PP-NRS will be completed by the caregiver. For subjects ages ≥12 years, the PP-NRS will be completed by the subject.

EQ-5D-5L and EQ-5D-Y: The EQ-5D-5L was introduced by the EuroQol Group in 2009 to improve the instrument's sensitivity and to reduce ceiling effects, as compared to the EQ-5D-3L. The EQ-5D-5L essentially consists of 2 pages: the EQ-5D descriptive system and the EQ visual analogue scale. The descriptive system comprises five dimensions: mobility, self-care, usual activities, pain/discomfort and anxiety/depression. Each dimension has 5 levels: no problems, slight problems, moderate problems, severe problems and extreme problems. The subject is asked to indicate his/her health state by ticking the box next to the most appropriate statement in each of the five dimensions. This decision results in a 1-digit number that expresses the level selected for that dimension. The digits for the five dimensions can be combined into a 5-digit number that describes the patient's health state. The EQ visual analogue scale records the patient's self-rated health on a vertical visual analogue scale, where the endpoints are labelled ‘The best health you can imagine’ and ‘The worst health you can imagine’. The visual analogue scale can be used as a quantitative measure of health outcome that reflects the patient's own judgement. The full version of EQ-5D-5L is available through the EuroQol Group. The EQ-5D-5L will be used in subjects ≥16 years and completed by the subject. The child-friendly EQ-5D version (EQ-SD-Y) was introduced by the EuroQol Group in 2009 as a more comprehensible instrument suitable for children and adolescents. The EQ-SD-Y is based on the EQ-5D-3L and essentially consist of 2 pages: the EQ-5D descriptive system and the EQ visual analogue scale. The EQ-SD-Y descriptive system comprises the following five dimensions: mobility, looking after myself, doing usual activities, having pain or discomfort and feeling worried, sad or unhappy. Each dimension has 3 levels: no problems, some problems and a lot of problems. The younger patient is asked to indicate his/her health state by ticking the box next to the most appropriate statement in each of the five dimensions. This decision results in a 1-digit number that expresses the level selected for that dimension. The digits for the five dimensions can be combined into a 5-digit number that describes the younger patient's health state. The EQ visual analogue scale records the younger patient's self-rated health on a vertical visual analogue scale where the endpoints are labelled “The best health you can imagine” and “The worst health you can imagine”. The visual analogue scale can be used as a quantitative measure of health outcome that reflects the younger patient's own judgement. The EQ-SD-Y will be used in subjects ≥8 to <16 years and completed by the subject. For subjects rolling over from DMVT-505-3101 or DMVT-505-3102, the EQ-5D-5L or EQ-SD-Y completed at the final visit of the parent study will be used as the Baseline value for this study. For subjects rolling over from DMVT-505-2104 or subjects enrolling directly into DMVT-505-3103, the EQ-5D-5L or EQ-5D-Y should be completed pre-dose at Baseline.

Dermatitis Family Impact Questionnaire: The DFI is a questionnaire that measures how much having a child with AD affects the quality of life of other members of the family. It is a 10-question tool that will be completed by the caregiver age 16 or over who has a pediatric subject <16 years old in the study. The same caregiver should be used to complete each questionnaire. For subjects rolling over from DMVT-505-3101 or DMVT-505-3102, the DFI completed at the final visit of the parent study will be used as the Baseline value for this study. For subjects rolling over from DMVT-505-2104 or subjects enrolling directly into DMVT-505-3103, the DFI should be completed pre-dose at Baseline.

Patient Satisfaction Questionnaire: For subjects ages 2 to <16 years, the Patient Satisfaction Questionnaire will be completed by the caregiver at the end of treatment (Week 48) or the Early Termination visit. For subjects ages ≥16 years, the Patient Satisfaction Questionnaire will be completed by the subject at the end of treatment (Week 48) or the Early Termination visit.

Optional Clinical Photography

Clinical photography may be performed in a subgroup of subjects at selected study sites. Informed consent/assent and photographic release will be required. The photographs may not be referred to by the Investigator at any subsequent study visit for the purposes of grading. Photographs will be taken of a representative area of the subject's disease area at the time points specified in the Schedule of Assessments. Photographs of the selected skin area will be taken in a standardized fashion (i.e., same camera, angle, background, distance).

What is claimed:

1. A method for treating atopic dermatitis in a subject in need thereof, comprising:
a. applying a thin layer of about 1.0% tapinarof topical cream composition to the affected areas of the subject once a day for an initial period of time of at least 8 weeks, until the subject has an IGA score of 0;
b. after the initial period of time, stop treating the subject with tapinarof for a remittive period of time of about 1 month to about 7 months, wherein the remittive period of time is the time wherein the subject maintains an IGA score <2; and
c. if, after the remittive period of time, the subject has an IGA score of ≥2, further applying a thin layer of 1.0% tapinarof topical cream composition to the affected areas of the subject once a day for a further period of time, until the subject has an IGA score of 0;
wherein:
the 1.0% tapinarof topical cream composition does not prolong the QTc interval in the subject while applying the 1.0% tapinarof topical cream,
the plasma concentration of tapinarof in the subject is below 50 pg/mL while applying the 1.0% tapinarof topical cream, and
the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

2. A method for treating atopic dermatitis in a subject in need thereof, wherein the subject has achieved an IGA score of 0 after applying a thin layer of about 1.0% tapinarof topical cream composition to the affected areas of the subject once a day for an initial period of time of about 8 weeks to 52 weeks, comprising:
a. stopping treatment of the subject with tapinarof for a remittive period of time of about 1 month to about 7 months, wherein the remittive period of time is the time wherein the subject has an IGA score <2, and
b. if, after the remittive period of time, the subject has an IGA score of ≥2, further applying a thin layer of 1.0% tapinarof topical cream composition to the affected areas of the subject once a day for a further period of time until the subject has an IGA score of 0;
wherein:
the 1.0% tapinarof topical cream composition does not prolong the QTc interval in the subject while applying the 1.0% tapinarof topical cream,
the plasma concentration of tapinarof in the subject is below 50 pg/mL while applying the 1.0% tapinarof topical cream, and
the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

3. A method for treating atopic dermatitis in a subject in need thereof, wherein the subject has an IGA score of at least 1 point lower than baseline but has not achieved an IGA score of 0 after applying a thin layer of about 1.0% tapinarof topical cream composition to the affected areas of the subject once a day for a period of time of about 8 weeks to about 52 weeks, comprising continuing to apply a thin layer of about 1.0% tapinarof topical cream composition to the affected areas of the subject once a day for an indefinite period of time, wherein the IGA score does not increase when applying a thin layer of about 1.0% tapinarof topical cream composition to the affected areas of the subject once a day;
wherein:
the 1.0% tapinarof topical cream composition does not prolong the QTc interval in the subject while applying the 1.0% tapinarof topical cream,
the plasma concentration of tapinarof in the subject is below 50 pg/mL while applying the 1.0% tapinarof topical cream, and
the subject selects a satisfaction rating of “agree” or “strongly agree” to one or more satisfaction questions selected from the group consisting of: 1) I can easily manage the atopic dermatitis with the study drug; 2) The time spent applying the study drug every day was acceptable and did not affect my everyday life; 3) I am satisfied with how well the study drug worked for my atopic dermatitis; 4) I have confidence in the study drug; 5) The study drug cleared my skin and kept my atopic dermatitis from coming back; 6) If the study drug was available by prescription, I would recommend it to other patients with atopic dermatitis; 7) If the study drug was available by a prescription, I would use it again or continue on it; 8) The study drug is easy to apply; 9) The study drug is not greasy; 10) The study drug quickly absorbs into my skin; 11) The study drug feels good on my skin; 12) I am satisfied with the look and feel of the study drug; 13) The study drug is more effective than other topical drugs I have used to treat my atopic dermatitis; 14) The study drug is easier to use than other topical drugs I have used to treat my atopic dermatitis; 15) I prefer the study drug to other topical drugs I have used to treat my atopic dermatitis; 16) The study drug is more effective than systemic drugs I have used to treat my atopic dermatitis; 17) The study drug is easier to use than systemic drugs I have used to treat my atopic dermatitis; and 18) I prefer the study drug to systemic drugs I have used to treat my atopic dermatitis.

4. The method of claim 1, wherein the atopic dermatitis is moderate or severe atopic dermatitis.

5. The method of claim 1, wherein the initial period of time is about 12 weeks to about 52 weeks.

6. The method of claim 1, wherein the remittive period is greater than 3 months and up to about 7 months.

7. The method of claim 1, wherein the remittive period is about 4 months.

8. The method of claim 1, wherein the further period of time is less than the initial period of time.

9. The method of claim 1, wherein the subject selects a satisfaction rating of “agree” or “strongly agree” to three or more of the satisfaction questions.

10. The method of claim 1, wherein the subject selects a satisfaction rating of “agree” or “strongly agree” to five or more of the satisfaction questions.

11. The method of claim 1, wherein the subject selects a satisfaction rating of “agree” or “strongly agree” to ten or more of the satisfaction questions.

12. The method of claim 2, wherein the atopic dermatitis is moderate or severe atopic dermatitis.

13. The method of claim 2, wherein the remittive period is greater than 3 months and up to about 7 months.

14. The method of claim 2, wherein the remittive period is about 4 months.

15. The method of claim 2, wherein the further period of time is less than the initial period of time.

16. The method of claim 2, wherein the subject selects a satisfaction rating of “agree” or “strongly agree” to three or more of the satisfaction questions.

17. The method of claim 2, wherein the subject selects a satisfaction rating of “agree” or “strongly agree” to five or more of the satisfaction questions.

18. The method of claim 3, wherein the atopic dermatitis is moderate or severe atopic dermatitis.

19. The method of claim 3, wherein the subject selects a satisfaction rating of “agree” or “strongly agree” to three or more of the satisfaction questions.

20. The method of claim 3, wherein the subject selects a satisfaction rating of “agree” or “strongly agree” to five or more of the satisfaction questions.
